(19) United States
(12) Patent Application Publication
(10) Pub. No.: US 2024/0327492 A1
(43) Pub. Date: Oct. 3, 2024

(54) Modified T Cell Receptors For The Prevention And Treatment Of Viral Infections And Cancer

(71) Applicant: NantCell, Inc., Culver City, CA (US)

(72) Inventors: Clifford Anders OLSON, Long Beach, CA (US); Hermes GARBÁN, Los Angeles, CA (US); Jay Gardner NELSON, Sherman Oaks, CA (US); Kayvan NIAZI, Agoura Hills, CA (US); Noe RODRIGUEZ, Culver City, CA (US); Peter Allan SIELING, Culver City, CA (US); Marcos SIXTO, Culver City, CA (US); Wendy M. HIGASHIDE, Rosemead, CA (US)

(21) Appl. No.: 18/580,246

(22) PCT Filed: Jul. 27, 2022
(86) PCT No.: PCT/US2022/074202

§ 371 Date: Jan. 18, 2024

Related U.S. Application Data
(60) Provisional application No. 63/227,195, filed on Jul. 29, 2021.

(51) Int. Cl. C07K 14/725; A61K 35/17; A61K 38/00; A61P 35/00; C07K 14/705; C07K 14/74; C12N 5/0783; C12N 15/63

(52) U.S. Cl. CPC C07K 14/7051; A61K 35/17; A61P 35/00; C07K 14/70521; C07K 14/70539; C12N 5/0636; C12N 5/0646; C12N 15/63; A61K 38/00; C07K 2319/03

(57) ABSTRACT

Modified T cell receptors, cells comprising the modified T cell receptors, nucleic acids encoding the modified T cell receptors, and methods for using the modified T cell receptors are contemplated. Modified T cell receptor comprising two peptide chains are disclosed herein. The two peptide chains may be the same or different, and each peptide chain comprises an extracellular domain comprising a variable region, a constant region, and a connecting peptide, a transmembrane domain, and an intracellular domain comprising a CD28 region and a CD3 ξ ITAM region. Nucleic acids encoding the modified T cell receptors, and vectors comprising the nucleic acids are also disclosed. Additionally, a method for treating cancer and/or a viral infection is disclosed comprising administering a cell comprising the modified T cell receptor.

### CROSS-REFERENCE TO RELATED APPLICATIONS

This application claims the benefit of U.S. Provisional Application No. 63/227,195 filed Jul. 29, 2021. The entire disclosure of the above application is incorporated herein by reference.

### REFERENCE TO A SEQUENCE LISTING

The present disclosure contains references to amino acid sequences and nucleic acid sequences which have been submitted concurrently herewith as the sequence listing xml file entitled “17768IB-01-WO-POA_seq_listing.xml.” file size 107 KiloBytes (KB), created on Jul. 25, 2022. The aforementioned sequence listing is hereby incorporated by reference in its entirety pursuant to 37 C.F.R. § 1.52 (e) (5).

### FIELD OF THE INVENTION

The present disclosure relates to modified T cell receptors (TCRs) that can be administered to subjects for the prevention and/or treatment of viral infections and/or cancer.

### BACKGROUND

The background description includes information that may be useful in understanding the compositions and methods described herein. It is not an admission that any of the information provided herein is prior art or relevant to the compositions and methods, or that any publication specifically or implicitly referenced is prior art.

TCRs are transmembrane proteins located on the surface of T cells which recognize antigens presented by major histocompatibility complex (MHC) I or II molecules from antigen presenting cells (APCs). Signaling through the T cell receptor with proper co-stimulation initiates a signaling pathway that activates the T cell to respond to an antigen (e.g. through the release of pro-inflammatory cytokines by helper CD4⁺ T cells or initiation of cell lysis by cytotoxic CD8⁺ T cells).

Cancers and viruses can escape T cell-mediated immune responses by mitigating TCR signaling, thereby downregulating the T cell response. Modifying a TCR to promote a T cell response can improve the host's immune response to a cancer or a viral infection.

T-Cell Receptor (TCR) molecules function in cellular contexts as dimers. Transgenically modifying T cell TCRs requires adding genes for each monomeric unit in the dimer. Because, however. T cells already contain natural TCRs, it is possible for the transgenic TCR monomers to heterodimerize with the natural TCRs. These hybrid TCRs can give rise to off-target effects in the transgenic T cells, wherein the effects of transgenic and/or endogenous TCR are reduced or eliminated by cross-binding of their respective peptide chains (Govers & al. (2010) Trends Mol. Med. 16 (2): 77-87). Thus, there remains a need to provide modified T cell receptors to enhance an immune response against specific antigens (e.g., antigens from cancer cells or viruses) for the treatment of cancer and/or viral infections.

### SUMMARY

Disclosed herein are modified TCRs that can be used to treat and/or prevent viral infections and/or cancer. The modified TCRs are heterodimers comprising two different peptide chains. The individual peptide chains of the modified TCRs each comprise an extracellular domain, a transmembrane domain, and an intracellular domain. The extracellular domain comprises a variable region, a constant region, and a connecting peptide, wherein the variable region and the constant region are attached via a linker. The connecting peptide is located between the constant region and the transmembrane domain. In some embodiments, the intracellular domain comprises a CD28 region and a CD3ξ ITAM region.

Also disclosed are methods for expressing a functional modified TCR into a T cell, wherein the peptide fragments of the modified TCR self-assemble on the cell surface with each other and not with endogenous TCR peptides also expressed on the T cell surface. The modified TCR can be genetically engineered to express a variable region comprising α and β chains with specificity for an HLA presented peptide. The modified TCR can be genetically engineered to express a variable region comprising an Ig variable domain with specificity for a tumor specific antigen.

Also disclosed herein are cells comprising the modified TCR.

Also disclosed herein are nucleic acids encoding the modified TCR and vectors comprising the nucleic acid encoding the modified TCR

Also disclosed herein are methods for the prevention and/or treatment of cancer or a viral infection in a patient in need thereof, the method comprising administering a therapeutically effective amount a pharmaceutical composition comprising a the modified TCR, a nucleic acid encoding the modified TCR, or a cell comprising the modified TCR to the patient.

Various objects, features, aspects, and advantages will become more apparent from the following detailed description of preferred embodiments, along with the accompanying drawings.

### BRIEF DESCRIPTION OF THE DRAWINGS

FIG. 1 depicts an embodiment of a modified TCR of the present disclosure comprising a heterodimer of peptide chains P-NR-025 and P-NR-026.

FIG. 2 depicts another embodiment of a modified TCR of the present disclosure comprising a heterodimer of peptide chains P-NR-027 and P-NR-028.

FIGS. 3A-3D depict plasmid constructs encoding particular peptide chains of the modified TCRs of the present disclosure. FIG. 3A depicts a plasmid encoding the peptide chain p-NR-025.

FIG. 3B depicts a plasmid encoding the peptide chain p-NR-026. FIG. 3C depicts a plasmid encoding the peptide chain p-NR-027. FIG. 3D depicts a plasmid encoding the peptide chain p-NR-028.

FIGS. 4A-4E show the results of a killing assay of activated natural killer (aNK) cells transfected with plasmid constructs encoding peptide chains of the modified TCRs of the present disclosure. FIGS. 4A and 4B show the target cell lysis by aNK cells transfected with the modified TCR P-NR-025+P-NR-026 and P-NR-027+P-NR-028, respectively. FIGS. 4C-4E show the target cell lysis of positive and negative control aNK cells.

FIG. 5 depicts the chimeric TCR expression in aNK (NK92) cells transfected with plasmid constructs encoding peptide chains of chimeric TCR P-NR-025+P-NR-026, P-NR-025+PWH295, PWH305+PWH 308, and PWH303+PWH308.

FIG. 6 shows the results of a killing assay in aNKs expressing wild type and chimeric TCRs shown in FIG. 5.

FIG. 7 depicts chimeric TCR expression in aNKs transfected with plasmid constructs encoding peptide chains of chimeric TCR PWH305+PWH308 and PWH303+PWH308.

FIG. 8 shows the results of a killing assay in aNKs expressing wild type and chimeric TCRs shown in FIG. 7.

### DETAILED DESCRIPTION

### I. Definitions

The following definitions refer to the various terms used above and throughout the disclosure.

“T cell receptor” or “TCR” refers to a dimeric polypeptide that is typically found on the surface of T cells. Each peptide chain of a TCR generally comprises an extracellular domain comprising a variable region and a constant region, a transmembrane domain, and an intracellular domain. The variable region is the portion of the TCR that interacts with the antigen presented by the MHC. The constant region is the area in each of the two peptides wherein the two peptide chains are covalently linked by a disulfide bond. The intracellular domain generally comprises a CD35, which comprises one or more immunoreceptor tyrosine-based activation motifs (ITAMs). The ITAM mediates the binding of the variable region to the appropriate intracellular signaling pathways.

“Encoding” when used in reference to a nucleic acid conveys that when transcription is initiated from the nucleic acid in a cell, the transcript produced would be translated into a given protein. That is to say, a nucleic acid “encodes” a peptide when the codon triplets of tRNA would produce the polypeptide from the nucleic acid according to the ordinary workings of transcription and translation in the cell.

“Effective amount” or “therapeutically effective amount” refers to the amount and/or dosage, and/or dosage regime of one or more agent(s) necessary to bring about the desired result e.g., an amount sufficient to prevent a viral infection in a subject, an amount sufficient to reduce the occurrence of a viral infection in a subject, and/or an amount sufficient to treat a viral infection in a subject. Alternatively, the effective amount or therapeutically effective amount refers to the amount and/or dosage and/or dosage regime sufficient to reduce the occurrence of a cancer in a subject, and/or an amount sufficient to treat a cancer in a subject.

“Cancer” refers to one or more conditions comprising the development of tumors, neoplasms, or otherwise unwanted, abnormal, and/or uncontrolled cellular growth in a patient's body, tissue, or organ. In certain embodiments, the cancer is selected from the group consisting of bladder cancer, bone cancer, brain cancer (including medulloblastoma, meningioma, neuroblastoma), breast cancer, cancer of the central nervous system, cervical cancer, colon cancer, colorectal cancer, esophageal cancer, eye cancer, gall bladder cancer, head and neck cancer, gastric cancer, HIV/AIDS related cancer, kidney cancer, leukemia. (including acute lymphoblastic leukemia (ALL), acute myeloid leukemia (AML), B cell leukemia (BCL), chronic lymphocytic cancer (CLL), chronic myeloid leukemia (CML), and chronic T cell lymphocytic leukemia (CTLL)), liver cancer, lung cancer (including non-small cell and small cell), lymphoma (including non-Hodgkin lymphoma and Hodgkin lymphoma), melanoma, multiple myeloma, nasopharyngeal cancer, oral cancer (including cancer of the mouth, tongue, salivary glands, or gums), neuroendocrine cancer, ovarian cancer, pancreatic cancer, prostate cancer, retinoblastoma, sarcoma, skin cancer, stomach cancer, testicular cancer, thyroid cancer, uterine cancer, and vaginal cancer. In a particular embodiment, the cancer is bladder cancer, breast cancer, colon cancer, or pancreatic cancer.

“Identical” or percent “identity.” in the context of two or more nucleic acids or polypeptide sequences, refer to two or more sequences or subsequences that are the same or have a specified percentage of amino acid residues or nucleotides that are the same, when compared and aligned for maximum correspondence over a comparison window. The degree of amino acid or nucleic acid sequence identity for purposes of the present disclosure is determined using the BLAST algorithm, described in Altschul et al. (199) J. Mol. Biol. 215:403 10, which is publicly available through software provided by the National Center for Biotechnology Information (at the web address www.ncbi.nlm.nih.gov). This algorithm identifies high scoring sequence pairs (HSPS) by identifying short words of length W in the query sequence, which either match or satisfy some positive-valued threshold score T when aligned with a word of the same length in a database sequence. T is referred to as the neighborhood word score threshold (Altschul et al., supra.). Initial neighborhood word hits act as seeds for initiating searches to find longer HSPs containing them. The word hits are then extended in both directions along each sequence for as far as the cumulative alignment score can be increased. Cumulative scores are calculated for nucleotides sequences using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below due to the accumulation of one or more negative-scoring residue alignments; or the end of either sequence is reached. For determining the percent identity of an amino acid sequence or nucleic acid sequence, the default parameters of the BLAST programs can be used. For analysis of amino acid sequences, the BLASTP defaults are: word length (W), 3; expectation (E). 10; and the BLOSUM62 scoring matrix. For analysis of nucleic acid sequences, the BLASTN program defaults are word length (W), 11; expectation (E), 10; M=5; N=−4; and a comparison of both strands. The TBLASTN program (using a protein sequence to query nucleotide sequence databases) uses as defaults a word length (W) of 3, an expectation (E) of 10, and a BLOSUM 62 scoring matrix. (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Nat'l. Acad. Sci. USA 90:5873-87). The smallest sum probability (P (N)), provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01.

“Viral infection” refers to a condition in which a virus has entered a host, such as a patient, and replicates. A viral infection does not require that the host presents symptoms of the viral infection. The term “virus” is not particularly limited and refers to both DNA and RNA viruses. The DNA virus may be a single- or double-stranded virus and may belong to any family of DNA viruses, including, but not limited to, herpesviridae, adenoviridae, polyomavididac, and poxviridae. Particular embodiments of DNA viruses include the human herpesvirus and varicella zoster virus. The RNA virus may also be single- or double-stranded and may belong to any family of RNA viruses, including, but not limited to, reoviridae, coronaviridae, picornaviridae, flaviviridae, hepeviridac, togaviridae, filoviridae, paramyxoviridae, pneumoviridae. rhabdoviridae, hantaviridae, and orthomyxoviridae. Particular embodiments of RNA viruses include rotavirus, coronavirus, SARS virus, poliovirus, rhinovirus, hepatitis A virus, yellow fever virus, west nile virus, hepatitis C virus, dengue fever virus, zika virus, rubella virus, sindbis virus. Chikungunya virus, Ebola virus, Marburg virus, measles virus, mumps virus, respiratory syncytial virus, rabies virus, influenza virus A, influenza virus B, influenza virus C, and influenza virus D. In some embodiments, the virus is human immunodeficiency virus.

“Subject,” “individual.” and “patient” interchangeably refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In certain embodiments, the subject can be human (e.g., adult male, adult female, adolescent male, adolescent female, male child, female child) under the care of a physician or other health worker. In certain embodiments the subject may not be under the care of a physician or other health worker.

“Treat” and “treatment” each refer to a method for reducing, inhibiting, or otherwise ameliorating an infection by administering a therapeutic to a subject in need of treatment. In some embodiments, the subject in need of treatment may include a subject having, diagnosed as having, or suspected to have an infection, such as a viral infection. In a particular embodiment, treat or treatment includes administering a therapeutic agent to a subject having, diagnosed as having, or suspected of having a disease, disorder, or condition (e.g., cancer or a viral infection). In some embodiments, the subject may be asymptomatic. Treatment includes administration of a modified TCR, a cell comprising the modified TCR, a nucleic acid encoding the modified TCR, and/or a vector comprising the nucleic acid encoding the modified TCR.

“Concomitant” or “concomitantly” includes administering an agent (e.g., a modified TCR, a cell comprising the modified TCR, and/or nucleic acid encoding the modified TCR) in the presence of an additional agent. Concomitant administration in a therapeutic treatment method includes methods in which a first, second, third, or additional agents are co-administered. Concomitant administration also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may administer to the subject a second agent, and the administering steps may be executed at the same time, or nearly the same time. The actor and the subject may be the same entity (e.g., human). Thus, the term embraces both simultaneous administration and substantially simultaneous administration, i.e., at about the same time.

II. Modified T cell Receptor

The modified TCR of the present invention relates to a dimeric polypeptide based on a TCR structure. In particular, the modified TCR comprises two peptide chains, each of which comprise an extracellular domain (comprising a variable region, a constant region, and a connecting peptide), a transmembrane domain, and an intracellular domain. In a specific embodiment, the variable region and constant region are attached via a linker. In another specific embodiment, the connecting peptide is located between the constant region and the transmembrane domain. In a further specific embodiment, the two peptide chains are connected to each other by a disulfide bond between the connecting peptides of each peptide chain.

The extracellular domain comprises a variable region, a constant region, and a connecting peptide. The exact sequence of the variable region is not particularly limited except that it is capable of recognizing an antigen presented on an MHC molecule. By convention the variable region on one of the modified TCR peptide chains may be called “Vα” and the variable region on the other peptide chain may be termed “VB.” In some embodiments, the variable regions of both peptide chains are the same. In alternative embodiments, the variable regions on each of the peptide chains are different. In a particular embodiment, the Vα comprises the sequence of SEQ ID NO: 15 (Vα-1) or SEQ ID NO: 30 (Vα-2). In another embodiment, the VB comprises the sequence of SEQ ID NO: 16 (VB-1) or SEQ ID NO: 31 (VB-2). Alternatively, the variable region comprises a sequence having at least 70% sequence identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to SEQ ID NO: 15, SEQ ID NO: 16, SEQ ID NO: 30, or SEQ ID NO: 31. In certain embodiments, the variable region comprises a sequence having at least 70% sequence identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to SEQ ID NO: 15 or SEQ ID NO: 30, but 100% identity to any or all of three complementarity determining regions (CDRs) of SEQ ID NO: 15 or SEQ ID NO: 30. In certain embodiments, the variable region comprises a sequence having at least 70% sequence identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to SEQ ID NO: 16 or SEQ ID NO: 31, but 100% identity to any or all of three complementarity determining regions (CDRs) of SEQ ID NO: 16 or SEQ ID NO: 31.

The constant region represents a peptide sequence between the variable region and the connecting peptide. In a specific embodiment, the constant region comprises an immunoglobulin (Ig) domain or a coiled-coil domain. In some embodiments, the constant regions of both peptide chains are the same. In alternative embodiments, the constant regions of each of the peptide chains is different.

In a particular embodiment the constant region is an Ig domain. The Ig domain is not particularly limited and may include IgA, IgD, IgE, IgG, and IgM. The constant region may comprise Ig-Cκ, IgG-CH-1, or IgM-CH-1. In a specific embodiment the Ig-Cκ comprises the sequence of SEQ ID NO: 17. In another embodiment, the IgG-CH-1 comprises the sequence of SEQ ID NO: 18 (IgG-CH-1a) or SEQ ID NO: 32 (IgG-CH-1b). In yet another embodiment, the IgM-CH-1 comprises the sequence of SEQ ID NO: 33. In a particular embodiment, the constant region of one peptide chain of the modified TCR comprises the sequence of Ig-Cκ and the constant region of the other peptide chain of the modified TCR comprises the sequence of Ig-CH-1, such as IgG-CH-1a, IgG-CH-1b, and IgM-CH-1. Alternatively, the constant region comprises a sequence having at least 70% sequence identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to SEQ ID NO: 17, SEQ ID NO: 18, SEQ ID NO: 32, or SEQ ID NO: 33.

In an alternative particular embodiment, the constant region is a coiled-coil domain such as a WinZip domain. WinZip domains have been described. See, for example, U.S. Pat. No. 6,897,017, the which is incorporated by reference herein in its entirety. In a specific embodiment, the WinZip domain is selected from the group consisting of WinZip-A2 (corresponding to SEQ ID NO: 20) and WinZip-B1 (corresponding to SEQ ID NO: 19). In a particular embodiment, the constant region of one peptide of the modified TCR comprises WinZip-A2 and the constant region of the other peptide of the modified TCR comprises WinZip-B1.

The linker that links the variable region and the constant region may be a flexible linker. In a particular embodiment, the linker comprises the amino acid sequence of GGSGG (SEQ ID NO: 2).

The connecting peptide conjoins the constant region to the transmembrane domain. In some embodiments, the connecting peptide comprises an amino acid sequence selected from the group consisting of GSG or GGCGG (SEQ ID NO: 1).

The extracellular region (comprising a variable region, a constant region, and a connecting peptide) of each peptide chain are covalently attached to a transmembrane domain. In some embodiments, the sequence of a transmembrane domain is selected from a human leukocyte antigen (HLA). In some embodiments, the transmembrane domains for both peptide chains are the same. In other embodiments, the transmembrane domains for both peptide chains are different.

In a particular embodiment, the transmembrane domain comprises HLA-DRA, HLA-DRB1, or HLA-DRB2. In a specific embodiment, the transmembrane domain comprises the amino acid sequence of SEQ ID NO: 21 (HLA-DRA), SEQ ID NO: 22 (HLA-DRB1), or SEQ ID NO: 34 (HLA-DRB2). Alternatively, the transmembrane domain comprises a sequence having at least 70% sequence identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to SEQ ID NO: 21. SEQ ID NO: 22, or SEQ ID NO: 34. In an embodiment, the transmembrane domain for one peptide chain comprises HLA-DRA and the transmembrane domain for the other peptide chain of the modified TCR comprises HLA-DRB, such as HLA-DRB1 or HLA-DRB2. In another particular embodiment, the transmembrane domain for one peptide chain of the modified TCR comprises the amino acid sequence of SEQ ID NO: 21 and the transmembrane domain for the other peptide chain of the modified TCR comprises the amino acid sequence of SEQ ID NO: 22 or SEQ ID NO: 34.

The peptide chains of the modified TCRs further comprise intracellular domains, each comprising a CD28 region and a CD3 (ITAM region. In a particular embodiment, the CD28 region comprises the amino acid sequence of SEQ ID NO:23. In another embodiment, the CD35 ITAM region comprises the amino acid sequence of SEQ ID NO: 24. Alternatively, the constant region comprises a sequence having at least 70% sequence identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to SEQ ID NO: 23 or SEQ ID NO: 24. In a still further embodiment, the intracellular domain of each peptide chain of the modified TCR comprises the amino acid sequence of SEQ ID NO: 23 and the amino sequence of SEQ ID NO: 24.

In an embodiment, each of the peptide chains of the modified TCR are the same as each other. In another embodiment, each of the two peptide chains in the modified TCR are different from each other.

Table 1 describes specific combinations of the peptide chains that dimerize to form a modified TCR.

TABLE 1

| | Extracellular Domain | | | Trans- | Intra- |
|---|---|---|---|---|---|
| | | | Con- | mem- | cel- |
| | Variable | Constant | necting | brane | lular |
| Name | Region | Region | Peptide | Domain | Domain |
| Pep- | Vα-1 | linker- | GGCGG | HLA- | CD28- |
| tide | | WinZip- | (SEQ ID | DRA | CD3ζ |
| 1 | | B1 | NO: 1) | | ITAM |
| Pep- | Vβ-1 | linker- | GGCGG | HLA- | CD28- |
| tide | | WinZip- | (SEQ ID | DRB1 | CD3ζ |
| 2 | | A2 | NO: 1) | | ITAM |
| Pep- | Vα-1 | GGSGG | GSG | HLA- | CD28- |
| tide | | (SEQ ID | | DRA | CD3ζ |
| 3 | | NO: 2)- | | | ITAM |
| | | Ig-Cκ | | | |
| Pep- | Vβ-1 | GGSGG | GSG | HLA- | CD28- |
| tide | | (SEQ ID | | DRB1 | CD3ζ |
| 4 | | NO: 2)- | | | ITAM |
| | | Ig-CH- | | | |
| | | 1a | | | |
| Pep- | Vα-2 | Ig-Cκ | GSG | HLA- | CD28- |
| tide | | | | DRA | CD3ζ |
| 5 | | | | | ITAM |
| Pep- | Vβ-2 | GGSGG | GSG | HLA- | CD28- |
| tide | | (SEQ ID | | DRB2 | CD3ζ |
| 6 | | NO: 2)- | | | ITAM |
| | | IgG-CH- | | | |
| | | 1a | | | |
| Pep- | Vβ-2 | IgG-CH- | GSG | HLA- | CD28- |
| tide | | 1b | | DRB2 | CD3ζ |
| 7 | | | | | ITAM |
| Pep- | Vβ-2 | IgM-CH- | GSG | HLA- | CD28- |
| tide | | 1 | | DRB2 | CD3ζ |
| 8 | | | | | ITAM |

Table 2 describes particular peptide chains that may homodimerize or heterodimerize with each other or other peptide chains comprising an extracellular domain (comprising a variable region, a constant region, and a connecting peptide), a transmembrane domain, and an intracellular domain.

TABLE 2

| Peptide Name | Amino Acid Sequence | Nucleic Acid Sequence |
|---|---|---|
| P-NR-027 | SEQ ID NO: 3 | SEQ ID NO: 12 |
| P-NR-028 | SEQ ID NO: 4 | SEQ ID NO: 14 |
| P-NR-025 | SEQ ID NO: 5 | SEQ ID NO: 8 |
| P-NR-026 | SEQ ID NO: 6 | SEQ ID NO: 10 |
| PWH308 | SEQ ID NO: 26 | SEQ ID NO: 35 |
| PWH295 | SEQ ID NO: 27 | SEQ ID NO: 36 |
| PWH303 | SEQ ID NO: 28 | SEQ ID NO: 37 |
| PWH305 | SEQ ID NO: 29 | SEQ ID NO: 38 |

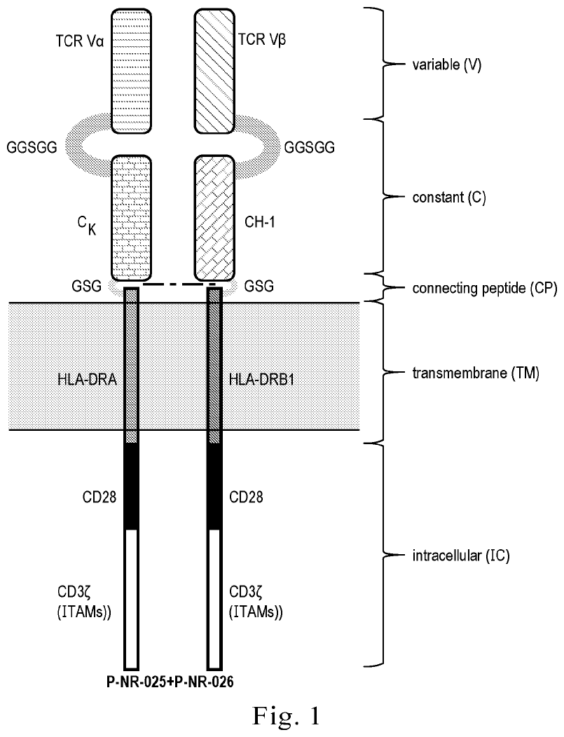

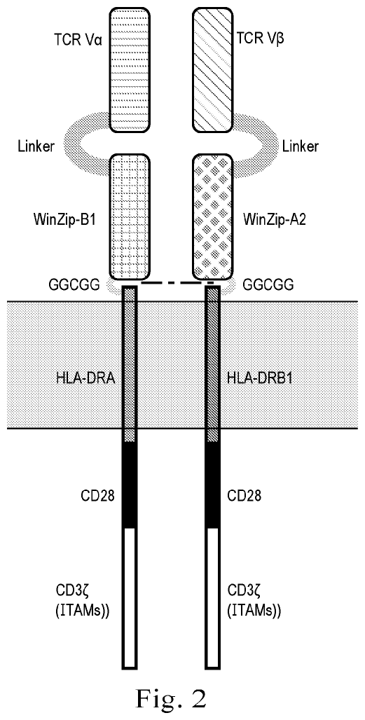

The peptide chains of Table 1 or Table 2 may form homodimers or heterodimers to generate the modified TCR. For example, P-NR-025 (SEQ ID NO: 5) and P-NR-026 (SEQ ID NO: 6) may dimerize to form a modified TCR (FIG. 1). Alternatively, P-NR-027 (SEQ ID NO: 3) and P-NR-028 (SEQ ID NO: 4) may dimerize to form another modified TCR (FIG. 2). Additional peptide chain combinations to form chimeric TCRs are shown in Table 3 below.

TABLE 3

| Ig-Cκ | Ig-CH-1 |
|---|---|
| P-NR-025 (SEQ ID NO: 5) | P-NR-026 (SEQ ID NO: 6) |
| PWH308 (SEQ ID NO: 26) | PWH295 (SEQ ID NO: 27) |
| PWH308 (SEQ ID NO: 26) | PWH303 (SEQ ID NO: 28) |
| PWH308 (SEQ ID NO: 26) | PWH305 (SEQ ID NO: 29) |

SEQ ID NOs: 15-24, 31-34, and 39-46 are offered only as examples of suitable portions of the peptide chain comprising the modified TCRs (i.e., specific variable region, constant region, connecting peptide, transmembrane domain, CD28 region, and CD35 ITAM region sequences) but many variations on these sequences are also useful for anti-viral or anti-cancer therapeutic purposes. For example, polypeptides having at least 70% sequence identity (i.e., at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity) to any one of SEQ ID NOs: 15-24, 31-34, and 39-46 are also useful for therapeutic purposes, provided that the molecule retains—broadly—the overall binding site, structure, and/or orientation of the individual SEQ ID NOs: 15-24, 31-34, and 39-46 molecules.

### III. Polynucleotides and Vectors

Contemporary molecular biologists know how to make nucleic acids that express the peptide chains described herein, and how to express such nucleic acids in cells to obtain the relevant proteins. Further embodiments provided herein include nucleic acids or polynucleotides that encode a peptide chain which comprise the modified TCR. For example, nucleic acids encoding the modified TCRs described herein are presented herein as SEQ ID NOs: 7 14 and 35-38. The ordinary molecular biologist knows how to alter the nucleotide sequence of SEQ ID NO: 8, 10, 12, 14, and 35-38 to encode peptide chains of SEQ ID NOs: 5, 6, 3, 4, and 26-29, respectively, and appropriate variants thereof (e.g., variants having at least 70% identity (e.g., at least 75%, at least 80%, at least 85%, at least 90%, at least 95%, at least 96%, at least 97%, at least 98%, or at least 99% to any one of SEQ ID NOs: 5, 6, 3, and 4). Non-limiting examples of nucleic acids encoding the peptide chains of SEQ ID NOs: 5, 6, 3, 4, and 26-29 are provided herein as SEQ ID NOs: 8, 10, 12, 14, and 35-38 respectively.

In certain embodiments, the nucleic acids described above can be expressed in a supporter cell line. Mammalian cell lines such as Chinese hamster ovary (CHO) cells or 293T cells are particularly suitable for these purposes. The proteins described herein are generally soluble, and will therefore be excreted from a producing cell unless they are modified for intracellular retention. Proteins produced in this manner can be purified from the culture medium. Where desired, the proteins may be tagged with (e.g.) a poly-histidine tag or other such commercially common tags to facilitate purification. Proteins produced and purified in this manner can then be administered to a subject in need thereof as described below.

Alternatively, the nucleic acids described above can be expressed in primary T cells, such as T cells obtained from peripheral blood, tumors, and/or lymph nodes. The primary T cells may be harvested and manipulated as is conventional in the art. The primary T cells may be from a subject having a condition treatable with the modified TCR described herein. Alternatively. the primary T cells may be from another subject having primary T cells which are immunocompatible with the subject to be treated.

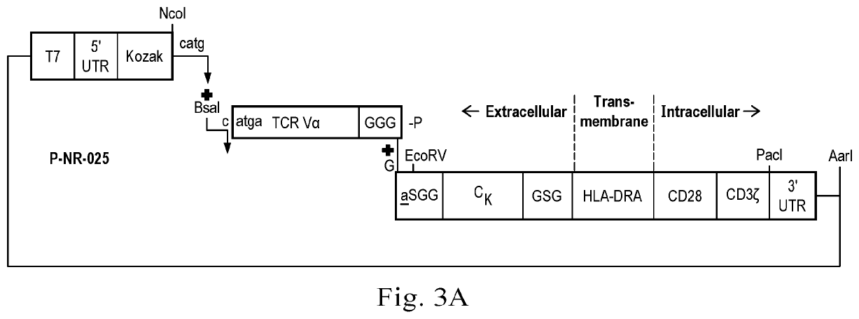

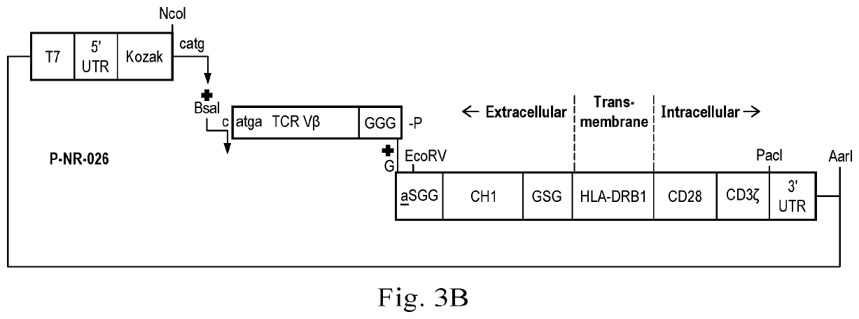

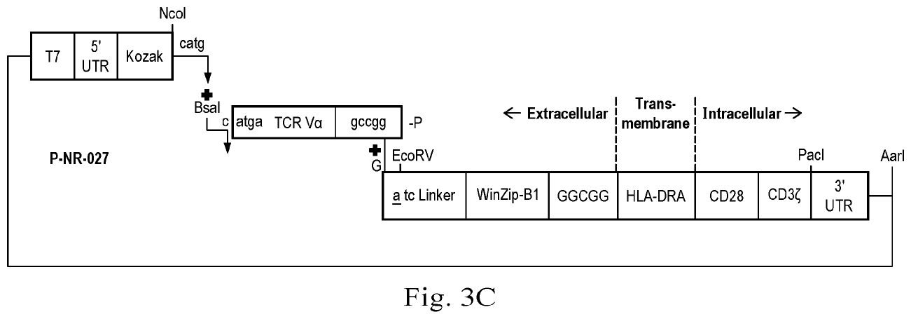

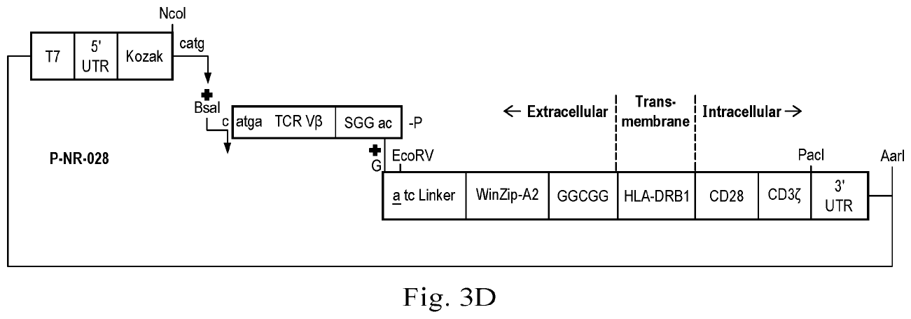

Additionally or alternatively, the nucleic acids described herein can be incorporated into a vector (e.g., a transfection vector or a viral transduction vector). Such vectors can then be transfected or transduced into the subject's own cells. In this way, the subject's own cells will produce the modified TCR. Non-limiting examples of vectors comprising the nucleic acids described above are provided herein as SEQ ID NOs: 7-14. The correlation of the vector with the peptide chain of the modified TCR is shown in Table 2, above. FIGS. 3A-3D show embodiments of SEQ ID NOs: 8, 10, 12, and 14, respectively.

In some embodiments, the nucleic acid encoding the modified TCR are incorporated into a cell. Such cells may translate the nucleic acid to encoding the modified TCR to express the TCR. The cells may be the subject's own cells (e.g., autologous cells) or cells from an appropriate donor (e.g., heterologous cells).

### IV. Methods of Prevention or Treatment

The proteins, peptides, cells, nucleic acids, and vectors described above can be used to treat and/or prevent and/or reduce the occurrence of viral infection and/or cancer. To treat and/or prevent a viral infection and/or cancer, the modified TCR, cells comprising the modified TCR, nucleic acids encoding the modified TCR, and vectors comprising the nucleic acids encoding the modified TCRs described herein can be administered to a subject in need thereof in a therapeutically effective amount. The subject may be symptomatic or asymptomatic. Therapeutically effective amounts of these modified TCRs include but are not limited to 1 μg of the modified TCR per kg of subject body weight, 5 μg/kg, 10 μg/kg, 50 μg/kg, 100 μg/kg, 500 μg/kg, 1 mg/kg, 5 mg/kg, 10 mg/kg, 50 mg/kg, 100 mg/kg, 500 mg/kg, and 1 mg/kg or more.

Where the modified TCR, cells comprising the modified TCR, nucleic acids encoding the modified TCR, and vectors comprising nucleic acids encoding the modified TCRs are administered, any suitable route of administration may be used, including but not limited to oral administration, intravenous injection, intramuscular injection, subcutaneous injection, and inhalation (e.g. aerosol inhalation). In a particular embodiment, the TCR is administered by modifying T cells or NK cells to express the TCR, and then by infusing the modified immune cells into the patient.

In a preferred embodiment, the modified TCR is transfected into autologous T cells derived from a patient with cancer of infectious disease. T cells may be derived from whole blood, a tumor, or a draining lymph node. In an embodiment, donor T cells may be used. The modified TCR described herein may be transfected into primary T cells as a nucleic acid, wherein the nucleic acid may be DNA or RNA in any suitable vector. The DNA vector may be an adenovirus. The nucleic acid may be RNA. The RNA may be in nanoparticle format such as is described in U.S. Pat. No. 11,141,377, which is incorporated herein by reference. Transfection may be performed by standard techniques, such as electroporation (for example as described in U.S. Pat. No. 11,377,652 and US 2022/0025402, both of which are incorporated herein by reference) or by using the MaxCyte™ system (Rockville. USA). Autologous T cells thus transfected may be ex vivo enriched and expanded. For example, CD3 enriched T cells may be expanded in Immunocult™ (StemCell Technologies, Cambridge. USA) and IL-2. T cells may be administered to the patient in therapeutically effective amounts. In some embodiments, the composition comprising the T cells manufactured by the methods described herein may be administered at a dosage of 102 to 10¹² cells/kg body weight. 102 to 10¹⁰ cells/kg body weight. 105 to 10⁹ cells/kg body weight, 105 to 10⁸ cells/kg body weight, 105 to 10⁷ cells/kg body weight, 107 to 10⁹ cells/kg body weight, or 107 to 10⁸ cells/kg body weight, including all integer values within those ranges. The number of T cells will depend on the therapeutic use for which the composition is intended.

Where a nucleic acid encoding the modified TCR is to be transfected into a cell, such as a T cell, any suitable amount can be transfected into a cell, including (but not limited to) 10 ng, 50 ng, 100 ng. 500 ng, 1 μg, 5 μg, 10 μg, 50 μg. 100 μg, 500 μg, 1 mg. 5 mg, 10 mg, 50 mg, 100 mg, and 500 mg or more. To transfect subject cells with polynucleotides as described herein, it will be useful to extract cells from the subject, transfect them according to known techniques, and then transfuse the transfected cells back into the subject. Electroporation is a particularly suitable transfection method (see, e.g., WO 20/14264 & WO 21/07315, each of which are herein incorporated by reference in their entireties). Particularly suitable cells include cells circulating throughout the body, such as circulating lymphocytes (e.g., T cells. NK cells).

Where a nucleic acid is to be transduced, a viral vector can be administered directly to the subject, or cells can be extracted for transduction and re-transfusion. The viral vector can be administered to the subject by any suitable route of administration, including but not limited to intravenous injection, intramuscular injection, subcutaneous injection, and inhalation (e.g. aerosol inhalation).

Therapeutically effective virus amounts include but are not limited to 1×10⁷ viral particles (VPs), 5×10⁷ VPs, 1×10⁸ VPs, 5×10⁸ VPs, 1×10⁹ VPs, 5×10⁹ VPs, 1×10¹⁰ VPs, or more than 1×10¹⁰ VPs. Adenoviral vectors are particularly suitable for this purpose because of the large cargo capacity of the adenovirus. Suitable adenoviral vectors include those disclosed in WO 98/17783, WO 02/27007, WO 09/6479, & WO 14/31178, each of which is incorporated herein by reference in its entirety. Suitable methods for administering these adenoviral vectors are disclosed in WO 16/112188, which is herein incorporated by reference in its entirety.

The proteins, peptides, cells, nucleic acids, and vectors described above can be used to treat and/or prevent and/or reduce the occurrence of cancer in a patient. The cancer may be bladder cancer, bone cancer, brain cancer (including medulloblastoma, meningioma, neuroblastoma), breast cancer, cancer of the central nervous system, cervical cancer, colon cancer, colorectal cancer, esophageal cancer, eye cancer, gall bladder cancer, head and neck cancer, gastric cancer, HIV/AIDS related cancer, kidney cancer, leukemia, (including acute lymphoblastic leukemia (ALL), acute myeloid leukemia (AML), B cell leukemia (BCL), chronic lymphocytic cancer (CLL), chronic myeloid leukemia (CML), and chronic T cell lymphocytic leukemia (CTLL)), liver cancer, lung cancer (including non-small cell and small cell), lymphoma (including non-Hodgkin lymphoma and Hodgkin lymphoma), melanoma, multiple myeloma, nasopharyngeal cancer, oral cancer (including cancer of the mouth, tongue, salivary glands, or gums), neuroendocrine cancer, ovarian cancer, pancreatic cancer, prostate cancer, retinoblastoma, sarcoma, skin cancer, stomach cancer, testicular cancer, thyroid cancer, uterine cancer, and vaginal cancer. In a particular embodiment, the the patient may have bladder cancer, breast cancer, colon cancer, or pancreatic cancer.

The proteins, peptides, cells, nucleic acids, and vectors described above can be used to treat and/or prevent and/or reduce the occurrence of a viral infection in a patient. The virus may be either a DNA or an RNA virus. The patient may be suffering an infection from a DNA virus such as a single- or double-stranded virus. The DNA virus may belong to any family of DNA viruses, including, but not limited to, herpesviridae, adenoviridae, polyomavididae, and poxviridae. Alternatively, the patient may be suffering an infection from an RNA virus, such as a single- or double-stranded virus. The RNA virus may belong to any family of RNA viruses, including, but not limited to, reoviridae, coronaviridae, picornaviridae, flaviviridae, hepeviridae, togaviridae, filoviridae, paramyxoviridae, pneumoviridae, rhabdoviridae, hantaviridae, and orthomyxoviridae. In particular, the patient may be infected with rotavirus, coronavirus, SARS virus, poliovirus, rhinovirus, hepatitis A virus, yellow fever virus, west nile virus, hepatitis C virus, dengue fever virus, zika virus, rubella virus, sindbis virus, Chikungunya virus, Ebola virus, Marburg virus, measles virus, mumps virus, respiratory syncytial virus, rabies virus, influenza virus A, influenza virus B, influenza virus C, influenza virus D, and human immunodeficiency virus.

### Embodiments

Embodiment 1: A modified T cell receptor (TCR) comprising a first peptide chain and a second peptide chain, wherein each peptide chain comprises: an extracellular domain; a transmembrane domain; and an intracellular domain, wherein the extracellular domain comprises a variable region, a constant region, and a connecting peptide, wherein the variable region and the constant region are attached via a linker, wherein the constant region of the first peptide chain comprises an Ig-Cκ domain and the constant region of the second peptide chain comprises an Ig-CH-1 domain, and wherein either 1) the transmembrane domain of the first peptide chain comprises an HLA-DRA domain and the transmembrane domain of the second peptide chain comprises an HLA-DRB domain, or 2) the transmembrane domain of the first peptide chain comprises an HLA-DRB domain and the transmembrane domain of the second peptide chain comprises an HLA-DRA domain.

Embodiment 2: The TCR of embodiment 1, wherein the linker is a flexible linker.

Embodiment 3: The TCR of embodiment 1 or 2, wherein the Ig-CH-1 domain is IgG-CH-1a, IgG-CH-1b, or IgM-CH-1.

Embodiment 4: The TCR of any one of embodiments 1-3, wherein the HLA-DRB domain is HLA-DRB1 or HLA-DRB2.

Embodiment 5: The TCR of any one of embodiments 1-4, wherein the variable regions on each of the peptide chains are the same variable region.

Embodiment 6: The TCR of any one of embodiments 1-5, wherein the variable regions on each peptide chain are different from each other.

Embodiment 7: The TCR of any one of embodiments 1-6, wherein the intracellular domain comprises a CD28 region and a CD35 ITAM region.

Embodiment 8: The TCR of any one of embodiments 1-7, wherein the first peptide chain comprises: Ig-Cκ as the constant region, HLA-DRA as the transmembrane domain; and CD28 and CD3ξ as the intracellular domain.

Embodiment 9: The TCR of embodiment 8, wherein the first peptide chain comprises SEQ ID NO: 39.

Embodiment 10: The TCR of embodiment 9, wherein the first peptide chain comprises SEQ ID NO: 5.

Embodiment 11: The TCR of embodiment 8, wherein the first peptide chain comprises SEQ ID NO: 43.

Embodiment 12: The TCR of embodiment 11, wherein the first peptide chain comprises SEQ ID NO: 26.

Embodiment 13: The TCR of any one of embodiments 1-7, wherein the second peptide chain comprises: Ig-CH-1 as the constant region, HLA-DRB as the transmembrane domain, and CD28 and CD3ξ as the intracellular domain.

Embodiment 14: The TCR of embodiment 13, wherein the second peptide chain comprises SEQ ID NO: 40.

Embodiment 15: The TCR of embodiment 14, wherein the second peptide chain comprises SEQ ID NO: 6.

Embodiment 16: The TCR of embodiment 13, wherein the second peptide chain comprises SEQ ID NO: 44.

Embodiment 17: The TCR of embodiment 16, wherein the second peptide chain comprises SEQ ID NO: 27.

Embodiment 18: The TCR of embodiment 13, wherein the second peptide chain comprises SEQ ID NO: 45.

Embodiment 19: The TCR of embodiment 18, wherein the second peptide chain comprises SEQ ID NO: 28.

Embodiment 20: The TCR of embodiment 13, wherein the second peptide chain comprises SEQ ID NO: 46.

Embodiment 21: The TCR of embodiment 20, wherein the second peptide chain comprises SEQ ID NO: 29.

Embodiment 22: The TCR of any one of embodiments 1-21, wherein the first peptide chain comprises Ig-Cκ as the constant region, HLA-DRA as the transmembrane domain; and

CD28 and CD35 as the intracellular domain; and the second peptide chain comprises Ig-CH-1 as the constant region, HLA-DRB as the transmembrane domain, and CD28 and CD35 as the intracellular domain.

Embodiment 23: The TCR of embodiment 22, wherein the first peptide chain comprises SEQ ID NO: 39 and the second peptide chain comprises SEQ ID NO: 40.

Embodiment 24: The TCR of embodiment 23, wherein the first peptide chain further comprises SEQ ID NO: 15 and the second peptide chain further comprises SEQ ID NO: 16.

Embodiment 25: The TCR of embodiment 24, wherein the first peptide comprises SEQ ID NO: 5 and the second peptide chain comprises SEQ ID NO: 6.

Embodiment 26: The TCR of embodiment 22, wherein the first peptide chain comprises SEQ ID NO: 43 and the second peptide chain comprises SEQ ID NO: 44.

Embodiment 27: The TCR of embodiment 26, wherein the first peptide chain further comprises SEQ ID NO: 30 and the second peptide chain further comprises SEQ ID NO: 31.

Embodiment 28: The TCR of embodiment 27, wherein the first peptide comprises SEQ ID NO: 26 and the second peptide chain comprises SEQ ID NO: 27.

Embodiment 29: The TCR of embodiment 22, wherein the first peptide chain comprises SEQ ID NO: 43 and the second peptide chain comprises SEQ ID NO: 44.

Embodiment 30: The TCR of embodiment 29, wherein the first peptide chain further comprises SEQ ID NO: 30 and the second peptide chain further comprises SEQ ID NO: 31.

Embodiment 31: The TCR of embodiment 30, wherein the first peptide comprises SEQ ID NO: 26 and the second peptide chain comprises SEQ ID NO: 28.

Embodiment 32: The TCR of embodiment 22, wherein the first peptide chain comprises SEQ ID NO: 43 and the second peptide chain comprises SEQ ID NO: 44.

Embodiment 33: The TCR of embodiment 32, wherein the first peptide chain further comprises SEQ ID NO: 30 and the second peptide chain further comprises SEQ ID NO: 31.

Embodiment 34: The TCR of embodiment 33, wherein the first peptide comprises SEQ ID NO: 26 and the second peptide chain comprises SEQ ID NO: 29.

Embodiment 35: A cell comprising the TCR of any one of embodiments 1-34.

Embodiment 36: A nucleic acid encoding the TCR of any one of embodiments 1-34.

Embodiment 37: A vector comprising the nucleic acid of embodiment 36

Embodiment 38: A method for reducing the occurrence of or treating cancer or a viral infection in a patient in need thereof, the method comprising administering a pharmaceutical composition to the patient, wherein the pharmaceutical composition comprises a therapeutically effective amount of the modified TCR of any one of claims 1-34 or a nucleic acid encoding the modified TCR of any one of embodiments 1-34.

Embodiment 39: The method of embodiment 38 wherein the pharmaceutical comprises a vector that comprises the nucleic acid.

Embodiment 40: The method of embodiments 38 or 39, wherein the pharmaceutical composition comprises a cell comprising the modified TCR or a nucleic acid encoding the modified TCR.

Embodiment 41: The method of any one of claims 38-40, wherein the cancer is selected from the group consisting of bladder cancer, bone cancer, brain cancer, breast cancer, cancer of the central nervous system, cervical cancer, colon cancer, colorectal cancer, esophageal cancer, eye cancer, gall bladder cancer, head and neck cancer, gastric cancer, HIV/AIDS related cancer, kidney cancer, leukemia, liver cancer, lung cancer, lymphoma, melanoma, multiple myeloma, nasopharyngeal cancer, oral cancer, neuroendocrine cancer, ovarian cancer, pancreatic cancer, prostate cancer, retinoblastoma, sarcoma, skin cancer, stomach cancer, testicular cancer, thyroid cancer, uterine cancer, and vaginal cancer.

Embodiment 42: The method of any one of claims 38-40, wherein the viral infection is caused by a virus from a viral family selected from the group consisting of herpesviridae, adenoviridae, polyomavididae, poxviridae, reoviridae, coronaviridae, picornaviridae, flaviviridae, hepeviridae, togaviridae, filoviridae, paramyxoviridae, pneumoviridae, rhabdoviridae, hantaviridae, and orthomyxoviridae.

Embodiment 43: Use of the TCR of claim 1, the cell of claim 35, the nucleic acid of claim 36 or the vector of claim 37 for preventing or treating a cancer or a viral infection in a patient in need thereof.

### EXAMPLES

The following example is provided to further illustrate the invention disclosed herein but should not be construed as in any way limiting its scope.

### Example 1: Cloning of TCR Constructs

The HLA-DRA or HLA-DRB1 connecting peptide plus transmembrane (CP-TM) region DNA template was built by annealing long partially overlapping oligonucleotides (IDT). Each of these CP-TM sequences was fused by overlap extension PCR (OF-PCR) to a CD28 intracellular (IC) plus CD35 (IC) sequence obtained from a previously cloned template. DNA templates encoding extracellular constant domains from Ig-Cκ or Ig-CH-1 were obtained by PCR from a previously cloned pAO156 template. The extracellular constant domains composed of Linker-WinZip-B1 or Linker-WinZip-A2 coiled-coil domain were obtained as gBlock DNA fragments (IDT) encoding either Linker-WinZip-B1 or Linker-WinZip-A2. Each of these extracellular constant domains was fused by OE-PCR to either the HLA-DRA or HLA-DRB1 CP-TM plus CD28-CD35 intracellular sequences. These invariable-CP-TM-IC sequences were cloned into a multi-purpose expression vector (pRNi). Invariable-CP-TM-IC inserts were cloned by a blunt ligation at the 5′ end so as to regenerate an EcoRV site, and a PacI overlap at the 3′ end. The resulting invariable-CP-TM-IC constructs in pRNi have an Ncol restriction site directly upstream of the coding sequence. As such, any TCR Vα or VB sequence can be designed or amplified with a BsaI or Esp3I restriction site at the 5′ end and a blunt, phosphorylated 3′ end and cloned into one of the invariable-CP-TM-IC vectors digested with Ncol and EcoRV. FIGS. 3A-3D illustrate particular embodiments of the cloned modified TCR constructs.

### Example 2: mRNA Synthesis and Effector Cell Electroporation

The modified TCR constructs of Example 1 were inserted into an expression vector. The constructs were flanked upstream by a T7 promoter and a 5′ UTR and downstream by a 3′ UTR adopted from mouse hemoglobin alpha, and a short poly (A) followed by an AarI linearization site. This allows for T7-based in vitro transcription of the modified TCR after linearization of the final vectors P-NR-025, P-NR-026, P-NR-027, and P-NR-028 with AarI. Activated natural killer (aNK) cells were electroporated with in vitro transcribed and polyadenylated mRNA (NEB Cat Nos. E2040S and M0276S) at 3 μg mRNA per 3×10⁶ cells in 50 μL using the BIO-RAD Gene Pulser II with 2 mm-gap cuvettes. Electroporated aNKs were incubated overnight at 1×10⁶ cells per mL in full RPMI media (Corning RPMI 1640 with L-Glu. supplemented with 10% FBS and 1×PSA) in wells of a 6-well TC-treated plate at 37° C. and 5% CO₂.

### Example 3: Killing Assay

Target D3C6 cells (i.e., KG-1 cells in which all MHC-I alleles have been knocked out) stably expressing HLA-A2 were pulsed at 6.4×10⁵ cells/mL in 4.4 mL each with either 4 μg/mL of hCMV pp65 NLV peptide-HLA-A*0201-restricted (NLVPMVATV (SEQ ID NO:25), in DMSO) or an equivalent volume of DMSO. The pulsed target cells were incubated overnight in full IMDM media (ATCC Iscove's IMDM supplemented with 10% FBS and 1× PSA) at 37° C. and 5% CO₂ in T-25 flasks. After 20-24 hr from the electroporation, the aNK effector cells of Example 2 were washed in PBS (without calcium or magnesium) and resuspended in full RPMI 1640 media. The effector aNK were then counted and serially diluted to from 1×10⁵ to 6.25×10⁴ live effector cells per well and deposited into round-bottom 96-well plates. Unbound peptide or DMSO was removed from pulsed target cells by washing once with full RPMI media and twice with PBS. Washed target cells were resuspended in 2 mL PBS with 20 μL of Calcein-AM (Fisher Scientific Cat No. C3099) each and incubated at 37° C. and 5% CO₂ for 20 minutes with gentle shaking. Calcein-loaded target cells were washed with PBS and then with full RPMI before resuspending in 10 mL full RPMI and counted. Target cells were loaded to wells of the 96-well round bottom plate at 5×10³ live target cells per well together with their effector cells. Killing assay plates were centrifuged at 400×g for 5 min and incubated for 4 hours at 37° C. and 5% CO₂. After incubation, 22 μL of 9% (v/v) Triton X-100 (Sigma Aldrich) was added to maximum lysis control wells and plates incubated at room temperature for 5 minutes. Killing plates were centrifuged again and 100 μL supernatant transferred to 96-well Immuno assay plates for excitation at 485+20 nm and emission read at 528+20 nm wavelength. Each sample was plated in triplicate.

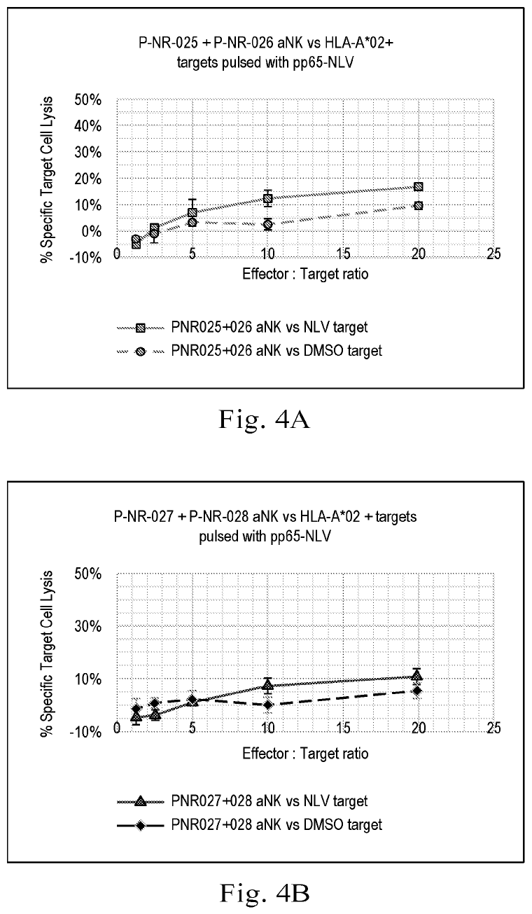

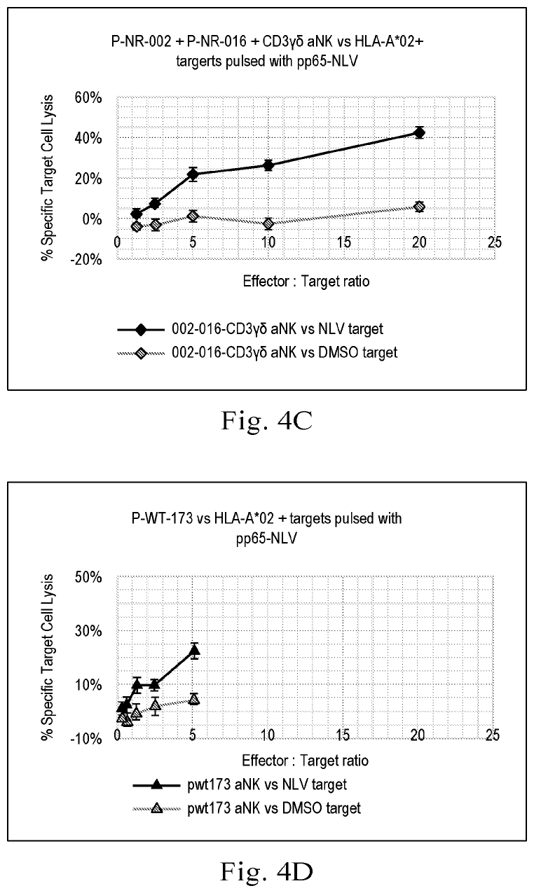

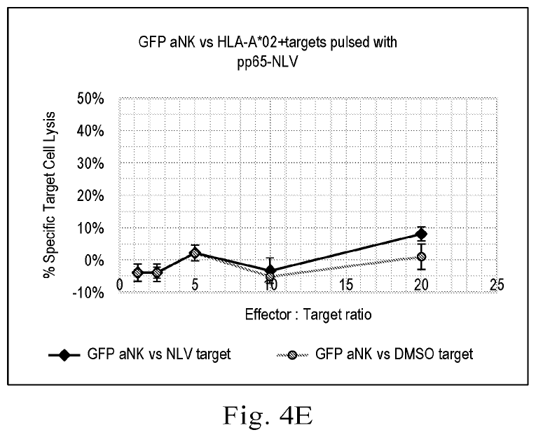

FIGS. 4A-4E demonstrate the % specific target cell lysis of HLA-A2 positive target cells pulsed with pp65-NLV or DMSO (control) and exposed to either TCRaβ-ITAM fusion-electroporated aNK cells or control aNK cells. “P-NR-025+P-NR-026 aNK” of FIG. 4A denotes the TCRaβ-ITAM fusions shown in FIG. 1. “P-NR-027+P-NR-028 aNK” of FIG. 4B denotes the TCRaβ-ITAM fusions shown in FIG. 2. “P-NR-002+P-NR-016+CD3γδ aNK” of FIG. 4C denotes wild-type TCRaβ containing the same anti-pp65-NLV-HLA-A2 variable domains as the other constructs, and co-electroporated with CD3γδ to serve as a positive killing control. “P-WT-173” of FIG. 4D is also a positive killing control, but stably expressing the same wild-type anti-pp65-NLV-HLA-A2 TCRaβ and CD3γδ. A negative control is depicted by aNK electroporated with GFP alone (FIG. 4F). P-NR-025+P-NR-026, P-NR-027+P-NR-028, P-NR-002+P-NR-016, and P-WT-173 all contained the same variable TCRaβ sequence pairs previously determined to bind to NLV peptide on HLA-A*02. Error bars only depict±standard deviation of technical replicates.

### Example 4: Chimeric TCR Expression and Cytotoxicity of P-NR-025+P-NR-026, P-NR-025+PWH295, PWH308+PWH305, or PWH308+PWH305 in Activated NK Cells

aNK (NK92) cells were washed with RPMI buffer and resuspended in RPMI at a concentration of 10⁷ cells/50 μL. 5 μg of mRNA encoding a first and second peptide chain were combined with 107 aNK cells in 50 μL RPMI to a 2 mm cuvette. The cuvettes were subjected to three 20 ms pulses of 200 V with a BioRad GenePulser II. Electroporated cells were transferred to culture media (Corning RPMI 1640 with L-Glu, supplemented with 10% FBS and 1×PSA) containing IL-2 and incubated overnight.

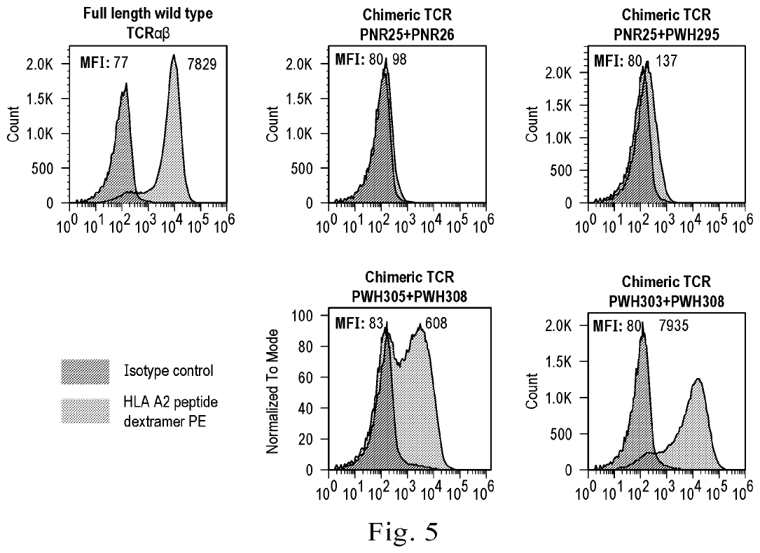

Following overnight incubation. 2×10⁵ cells from each sample were obtained and washed with a PBS/BSA/EDTA buffer. The cells were resuspended in 100 μL of the wash buffer. 5 μL of PE-HLA-A*0201, NLVPMVATV-PE, or HLA-A2 dextramer negative control were added to each sample. The samples were incubated at 4° C. for 20 minutes, washed with PBS/BSA/EDTA and resuspended in 200 μL. The cells were analyzed via flow cytometry. FIG. 5 depicts the expression of various chimeric TCRs in the electroporated aNKs.

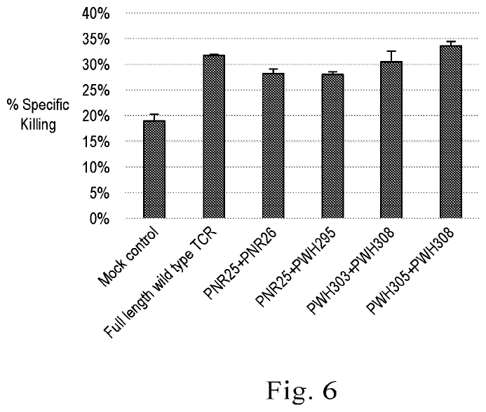

Following overnight incubation after electroporation, the aNKs were washed three times and incubated at a 10:1 (effector: target) ratio with HLA-A2 stable KG-1 cells stained with calcein AM. After a 4-hour incubation, the supernatant was obtained and analyzed for the fluorescence of calcein AM. FIG. 6 shows the % specific killing of the target cells by aNK cells expressing modified TCRs described herein.

### Example 5: Chimeric TCR Expression and Cytotoxicity of PWH308+PWH305 and PWH308+PWH305 in Primary T Cells

Human primary T cells were obtained from donor derived leukopacks (Charles River. Wilmington, USA). The peripheral blood mononuclear cells (PBMCs) were separated via ficoll gradient and washed with K100 buffer and resuspended in K100 at a concentration of 10⁷ cells/100 μL. CD3-enriched T cells were then expanded in ImmunoCult™ (StemCell Technologies, Cambridge, USA) and IL-2. 10 μg of mRNA encoding a first and second peptide chain were combined with 107 T cells in 100 μL K100 buffer to a 2 mm cuvette. The cells were electroporated according to the electroporation protocol described in U.S. Pat. Nos. 11,377,652 and 20,220,025402, both of which are incorporated herein by reference. Electroporated cells were transferred to culture media and incubated overnight.

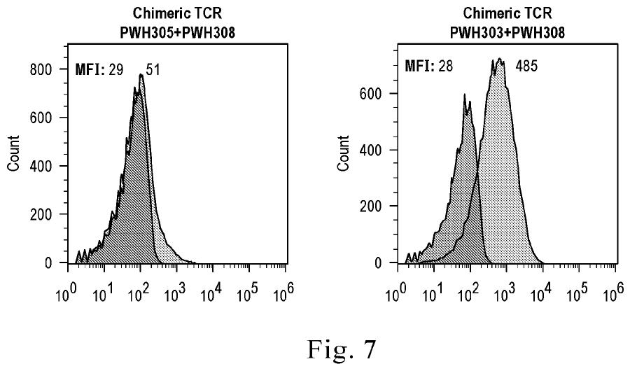

Following overnight incubation, 2×10⁵ cells from each sample were obtained and washed with a PBS/BSA/EDTA buffer. The cells were resuspended in 100 μL of the wash buffer. 5 μL of PE-HLA-A*0201, NLVPMVATV-PE, or HLA-A2 dextramer negative control were added to each sample. The samples were incubated at 4° C. for 20 minutes, washed twice with PBS/BSA/EDTA and resuspended in 200 μL. The cells were analyzed via flow cytometry. FIG. 7 depicts the expression of various chimeric TCRs in the electroporated primary T cells.

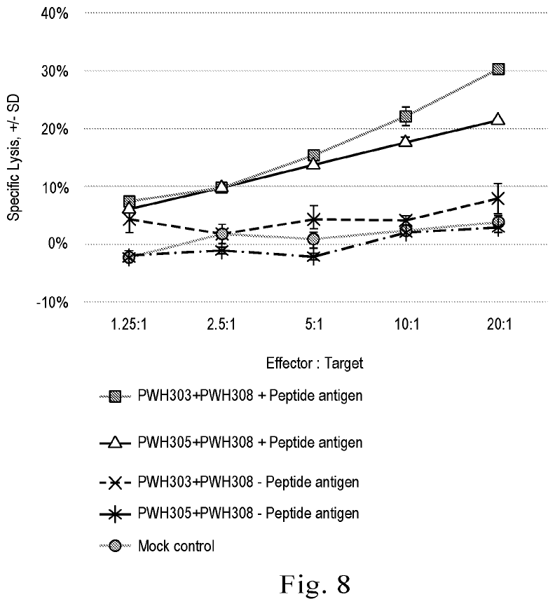

Following overnight incubation after electroporation, the primary T cells were washed three times and incubated at a 10:1 (effector: target) ratio with HLA-A2 stable KG-1 cells stained with calcein AM. After a 4 hour incubation, the supernatant was obtained and analyzed for the fluorescence of calcein AM. FIG. 8 shows the % specific killing of the target cells by primary T cells expressing modified TCRs described herein.

### Example 6: Transfection of Patient-Derived T Cells with Modified TCR

Patient T cells are derived from whole peripheral blood or isolated from a tumor or draining lymph nodes. The T cells are electroporated with a modified TCR as described herein. The electroporated T cells are grown ex vivo to a clinically efficacious number of cells and a therapeutically relevant number of cells are administered to the patient.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising.” “having.” “including.” and “containing” are to be construed as open-ended terms (i.e., meaning “including, but not limited to.”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Particular embodiments of this invention are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Sequence listing

| SEQ | | |
|---|---|---|
| ID | | |
| NO: | Description | Sequence |
| 1 | Linker | GGCGG |
| 2 | Linker | GGSGG |
| 3 | P-NR-027 | MKSLRVLLVILWLQLSWVWSQQKEVEQNSGPLSVPEGAIASLNCT |
| | | YSDRGSQSFFWYRQYSGKSPELIMSIYSNGDKEDGRFTAQLNKASQ |
| | | YVSLLIRDSQPSDSATYLCAVNVPLSYQLTFGKGTKLSVIPNAGSST |
| | | GSSTGPGSTSVDELQAEVDQLQDENYALKTKVAQLRKKVEKLASG |
| | | GCGGEFDAPSPLPETTENVVCALGLTVGLVGIIIGTIFIIRSKRSRLLH |
| | | SDYMNMTPRRPGPTRKHYQPYAPPRDFAAYRSRVKFSRSADAPAY |
| | | QQGQNQLYNELNLGRREEYDVLDKRRGRDPEMGGKPQRRKNPQE |
| | | GLYNELQKDKMAEAYSEIGMKGERRRGKGHDGLYQGLSTATKDT |
| | | YDALHMQALPPR |
| 4 | P-NR-028 | MLSLLLLLLGLGSVFSAVISQKPSRDICQRGTSLTIQCQVDSQVTMM |
| | | FWYRQQPGQSLTLIATANQGSEATYESGFVIDKFPISRPNLTFSTLTV |
| | | SNMSPEDSSIYLCSVAGTIDEQYFGPGTRLTVTESGGTSGSTSGTGST |
| | | TVAQLRERVKTLRAQNYELESEVQRLREQVAQLASGGCGGRARSE |
| | | SAQSKMLSGVGGFVLGLLFLGAGLFIRSKRSRLLHSDYMNMTPRRP |
| | | GPTRKHYQPYAPPRDFAAYRSRVKFSRSADAPAYQQGQNQLYNEL |
| | | NLGRREEYDVLDKRRGRDPEMGGKPQRRKNPQEGLYNELQKDKM |
| | | AEAYSEIGMKGERRRGKGHDGLYQGLSTATKDTYDALHMQALPP |
| | | R |
| 5 | P-NR-025 | MKSLRVLLVILWLQLSWVWSQQKEVEQNSGPLSVPEGAIASLNCT |
| | | YSDRGSQSFFWYRQYSGKSPELIMSIYSNGDKEDGRFTAQLNKASQ |
| | | YVSLLIRDSQPSDSATYLCAVNVPLSYQLTFGKGTKLSVIPNGGSGG |
| | | RTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQ |
| | | GLSSPVTKSFNRGECGSGEFDAPSPLPETTENVVCALGLTVGLVGIII |
| | | GTIFIIRSKRSRLLHSDYMNMTPRRPGPTRKHYQPYAPPRDFAAYRS |
| | | RVKFSRSADAPAYQQGQNQLYNELNLGRREEYDVLDKRRGRDPE |
| | | MGGKPQRRKNPQEGLYNELQKDKMAEAYSEIGMKGERRRGKGHD |
| | | GLYQGLSTATKDTYDALHMQALPPR |
| 6 | P-NR-026 | MLSLLLLLLGLGSVFSAVISQKPSRDICQRGTSLTIQCQVDSQVTMM |
| | | FWYRQQPGQSLTLIATANQGSEATYESGFVIDKFPISRPNLTFSTLTV |
| | | SNMSPEDSSIYLCSVAGTIDEQYFGPGTRLTVTEGGSGGASTKGPSV |
| | | FPLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVEP |
| | | KSCGSGRARSESAQSKMLSGVGGFVLGLLFLGAGLFIRSKRSRLLH |
| | | SDYMNMTPRRPGPTRKHYQPYAPPRDFAAYRSRVKFSRSADAPAY |
| | | QQGQNQLYNELNLGRREEYDVLDKRRGRDPEMGGKPQRRKNPQE |
| | | GLYNELQKDKMAEAYSEIGMKGERRRGKGHDGLYQGLSTATKDT |
| | | YDALHMQALPPR |
| 7 | P-NR-021 | AGCTAGCTTTAATACGACTCACTATAAGGAAATAAGAGAGAAA |
| | plasmid | AGAAGAGTAAGAAGAAATATAAGAGCCACCATGGGCTAGTACA |
| | | GATATCTGGTGGAAGAACTGTTGCGGCGCCATCAGTGTTCATCT |
| | | TTCCCCCTAGCGACGAGCAGCTGAAGAGTGGCACAGCTTCCGTG |
| | | GTCTGCCTGCTGAACAATTTCTACCCCCGGGAAGCCAAGGTGCA |
| | | GTGGAAAGTCGATAACGCTCTGCAGTCTGGAAATAGTCAGGAGT |
| | | CAGTGACTGAACAGGACAGCAAGGATTCCACCTATTCTCTGAGC |
| | | TCCACCCTGACACTGTCTAAAGCAGACTACGAGAAGCACAAAGT |
| | | CTATGCCTGTGAAGTCACTCACCAGGGTCTGTCTTCACCAGTCAC |
| | | CAAATCCTTCAATAGGGGGGAATGCGGCAGTGGTGAGTTTGATG |
| | | CTCCAAGCCCTCTCCCAGAGACTACAGAGAACGTGGTGTGTGCC |
| | | CTGGGCCTGACTGTGGGTCTGGTGGGCATCATTATTGGGACCAT |
| | | CTTCATCATCAGGAGTAAGAGGAGCAGGCTCCTGCACAGTGACT |
| | | ACATGAACATGACTCCCCGCCGCCCCGGGCCCACCCGCAAGCAT |
| | | TACCAGCCCTATGCCCCACCACGCGACTTCGCAGCCTATCGCTC |
| | | CAGAGTGAAGTTCAGCAGGAGCGCAGACGCCCCCGCGTACCAG |
| | | CAGGGCCAGAACCAGCTCTATAACGAGCTCAATCTAGGACGAA |
| | | GAGAGGAGTACGATGTTTTGGACAAGAGACGTGGCCGGGACCC |
| | | TGAGATGGGGGGAAAGCCGCAGAGAAGGAAGAACCCTCAGGAA |
| | | GGCCTGTACAATGAACTGCAGAAAGATAAGATGGCGGAGGCCT |
| | | ACAGTGAGATTGGGATGAAAGGCGAGCGCCGGAGGGGCAAGGG |
| | | GCACGATGGCCTTTACCAGGGTCTCAGTACAGCCACCAAGGACA |
| | | CCTACGACGCCCTTCACATGCAGGCCCTGCCCCCTCGCTGATTA |
| | | ATTAAGCTGCCTTCTGCGGGGCTTGCCTTCTGGCCATGCCCTTCT |
| | | TCTCTCCCTTGCACCTGTACCTCTTGGTCTTTGAATAAAGCCTGA |
| | | GTAGGAAGAAAAAAAAAAAAAAAAAAAAAAAGCAGGTGGCGG |
| | | CCGCAGGTAAGCCAGCCCAGGCCTCGCCCTCCAGCTCAAGGCGG |
| | | GACAGGTGCCCTAGAGTAGCCTGCATCCAGGGACAGGCCCCAG |
| | | CCGGGTGCTGACACGTCCACCTCCATCTCTTCCTCAGGTCTGCCC |
| | | GGGTGGCATCCCTGTGACCCCTCCCCAGTGCCTCTCCTGGTCGTG |
| | | GAAGGTGCTACTCCAGTGCCCACCAGCCTTGTCCTAATAAAATT |
| | | AAGTTGCATCATTTTGTTTGACTAGGTGTCCTTGTATAATATTAT |
| | | GGGGTGGAGGCGGGTGGTATGGAGCAAGGGGCCCAAGTTAACT |
| | | TGTTTATTGCAGCTTATAATGGTTACAAATAAAGCAATAGCATC |
| | | ACAAATTTCACAAATAAAGCATTTTTTTCACTGCATTCTAGTTGT |
| | | GGTTTGTCCAAACTCATCAATGTATCTTATCATGTCTGGATCCGC |
| | | TTCAGGCACCGGGCTTGCGGGTCATGCACCAGGTGCGCGGTCCT |
| | | TCGGGCACCTCGACGTCGGCGGTGACGGTGAAGCCGAGCCGCTC |
| | | GTAGAAGGGGAGGTTGCGGGGCGCGGAGGTCTCCAGGAAGGCG |
| | | GGCACCCCGGCGCGCTCGGCCGCCTCCACTCCGGGGAGCACGAC |
| | | GGCGCTGCCCAGACCCTTGCCCTGGTGGTCGGGCGAGACGCCGA |
| | | CGGTGGCCAGGAACCACGCGGGCTCCTTGGGCCGGTGCGGCGCC |
| | | AGGAGGCCTTCCATCTGTTGCTGCGCGGCCAGCCTGGAACCGCT |
| | | CAACTCGGCCATGCGCGGGCCGATCTCGGCGAACACCGCCCCCG |
| | | CTTCGACGCTCTCCGGCGTGGTCCAGACCGCCACCGCGGCGCCG |
| | | TCGTCCGCGACCCACACCTTGCCGATGTCGAGCCCGACGCGCGT |
| | | GAGGAAGAGTTCTTGCAGCTCGGTGACCCGCTCGATGTGGCGGT |
| | | CCGGGTCGACGGTGTGGCGCGTGGCGGGGTAGTCGGCGAACGC |
| | | GGCGGCGAGGGTGCGTACGGCCCGGGGGACGTCGTCGCGGGTG |
| | | GCGAGGCGCACCGTGGGCTTGTACTCGGTCATGGTGGCCTGCAG |
| | | AGTCGCTCTGTGTTCGAGGCCACACGCGTCACCTTAATATGCGA |
| | | AGTGGACCTGGGACCGCGCCGCCCCGACTGCATCTGCGTGTTTT |
| | | CGCCAATGACAAGACGCTGGGCGGGGTTTGTGTCATCATAGAAC |
| | | TAAAGACATGCAAATATATTTCTTCCGGGGACACCGCCAGCAAA |
| | | CGCGAGCAACGGGCCACGGGGATGAAGCAGCTGCGCCACTCCC |
| | | TGAAGATCCATCGTCTCCTAACAAGTTACATCACTCCTGCCCTTC |
| | | CTCACCCTCATCTCCATCACCTCCTTCATCTCCGTCATCTCCGTC |
| | | ATCACCCTCCGCGGCAGCCCCTTCCACCATAGGTGGAAACCAGG |
| | | GAGGCAAATCTACTCCATCGTCAAAGCTGCACACAGTCACCCTG |
| | | ATATTGCAGGTAGGAGCGGGCTTTGTCATAACAAGGTCCTTAAT |
| | | CGCATCCTTCAAAACCTCAGCAAATATATGAGTTTGTAAAAAGA |
| | | CCATGAAATAACAGACAATGGACTCCCTTAGCGGGCCAGGTTGT |
| | | GGGCCGGGTCCAGGGGCCATTCCAAAGGGGAGACGACTCAATG |
| | | GTGTAAGACGACATTGTGGAATAGCAAGGGCAGTTCCTCGCCTT |
| | | AGGTTGTAAAGGGAGGTCTTACTACCTCCATATACGAACACACC |
| | | GGCGACCCAAGTTCCTTCGTCGGTAGTCCTTTCTACGTGACTCCT |
| | | AGCCAGGAGAGCTCTTAAACCTTCTGCAATGTTCTCAAATTTCG |
| | | GGTTGGAACCTCCTTGACCACGATGCTTTCCAAACCACCCTCCTT |
| | | TTTTGCGCCTGCCTCCATCACCCTGACCCCCGCTGCGCGGGGGC |
| | | ACGTCAGGCTCACCATCTGGGCCGCCTTCTTGGTGGTATTCAAA |
| | | ATAATCGGCTTCCCCTACAGGGTGGAAAAATGGCCTTCTACCTG |
| | | GAGGGGGCCTGCGCGGTGGAGACCCGGATGATGATGACTGACT |
| | | ACTGGGACTCCTGGGCCTCTTTTCTCCACGTCCACGACCTCTCCC |
| | | CCTGGCTCTTTCACGACTTCCCCCCCTGGCTCTTTCACGTCCTCT |
| | | ACCCCGGCGGCCTCCACTACCTCCTCGACCCCGGCCTCCACTAC |
| | | CTCCTCGACCCCGGCCTCCACTGCCTCCTCGACCCCGGCCTCCAC |
| | | CTCCTGCTCCTGCCCCTCCCGCTCCTGCTCCTGCTCCTGTTCCACC |
| | | GTGGGTCCCTTTGCAGCCAATGCAACTTGGACGTTTTTGGGGTCT |
| | | CCGGACACCATCTCTATGTCTTGGCCCTGATCCTGAGCCGCCCG |
| | | GGGCTCCTGGTCTTCCGCCTCCTCGTCCTCGTCCTCTTCCCCGTC |
| | | CTCGTCCATGTGCCATGATGGCGGCCTGCAGCTGTGTTCGAGGC |
| | | CGCGCGTGTCACCTTAATATGCGAAGTGGACCTGGGACCGCGCC |
| | | GCCCCGACTGCATCTGCGTGTTCGAGTTCGCCAATGACAAGACG |
| | | CTGGGCGGGGAGATCCCCCTTATTAACCCTAAACGGGTAGCATA |
| | | TGCTTCCCGGGTAGTAGTATATACTATCCAGACTAACCCTAATTC |
| | | AATAGCATATGTTACCCAACGGGAAGCATATGCTATCGAATTAG |
| | | GGTTAGTAAAAGGGTCCTAAGGAACAGCGATCTGGATAGCATAT |
| | | GCTATCCTAATCTATATCTGGGTAGCATATGCTATCCTAATCTAT |
| | | ATCTGGGTAGCATAGGCTATCCTAATCTATATCTGGGTAGCATAT |
| | | GCTATCCTAATCTATATCTGGGTAGTATATGCTATCCTAATTTAT |
| | | ATCTGGGTAGCATAGGCTATCCTAATCTATATCTGGGTAGCATAT |
| | | GCTATCCTAATCTATATCTGGGTAGTATATGCTATCCTAATCTGT |
| | | ATCCGGGTAGCATATGCTATCCTCATGCATATACAGTCAGCATA |
| | | TGATACCCAGTAGTAGAGTGGGAGTGCTATCCTTTGCATATGCC |
| | | GCCACCTCCCAAGGAGATCTGTCGACATCGATGGGCGCGGGTGT |
| | | ACACTCCGCCCATCCCGCCCCTAACTCCGCCCAGTTCCGCCCATT |
| | | CTCCGCCTCATGGCTGACTAATTTTTTTTATTTATGCAGAGGCCG |
| | | AGGCCGCCTCGGCCTCTGAGCTATTCCAGAAGTAGTGAGGAGGC |
| | | TTTTTTGGAGGCCTAGGCTTTTGCAAAAAGCTAATTCGGCGTAAT |
| | | CTGCTGCTTGCAAACAAAAAAACCACCGCTACCAGCGGTGGTTT |
| | | GTTTGCCGGATCAAGAGCTACCAACTCTTTTTCCGAAGGTAACT |
| | | GGCTTCAGCAGAGCGCAGATACCAAATACTGTCCTTCTAGTGTA |
| | | GCCGTAGTTAGGCCACCACTTCAAGAACTCTGTAGCACCGCCTA |
| | | CATACCTCGCTCTGCTGAAGCCAGTTACCAGTGGCTGCTGCCAG |
| | | TGGCGATAAGTCGTGTCTTACCGGGTTGGACTCAAGAGATAGTT |
| | | ACCGGATAAGGCGCAGCGGTCGGGCTGAACGGGGGGTTCGTGC |
| | | ACACAGCCCAGCTTGGAGCGAACGACCTACACCGAACTGAGAT |
| | | ACCTACAGCGTGAGCTATGAGAAAGCGCCACGCTTCCCGAAGG |
| | | GAGAAAGGCGGACAGGTATCCGGTAAGCGGCAGGGTCGGAACA |
| | | GGAGAGCGCACGAGGGAGCTTCCAGGGGGAAACGCCTGGTATC |
| | | TTTATAGTCCTGTCGGGTTTCGCCACCTCTGACTTGAGCGTCGAT |
| | | TTTTGTGATGCTCGTCAGGGGGGCGGAGCCTATGGAAAAACGCC |
| | | AGCAACGCAAGCTAGAGTTTAAACTTGACAGATGAGACAATAA |
| | | CCCTGATAAATGCTTCAATAATATTGAAAAAGGAAAAGTATGAG |
| | | TATTCAACATTTCCGTGTCGCCCTTATTCCCTTTTTTGCGGCATTT |
| | | TGCCTTCCTGTTTTTGCTCACCCAGAAACGCTGGTGAAAGTAAA |
| | | AGATGCAGAAGATCACTTGGGTGCGCGAGTGGGTTACATCGAAC |
| | | TGGATCTCAACAGCGGTAAGATCCTTGAGAGTTTTCGCCCCGAA |
| | | GAACGTTTCCCAATGATGAGCACTTTTAAAGTTCTGCTATGTGGC |
| | | GCGGTATTATCCCGTATTGATGCCGGGCAAGAGCAACTCGGTCG |
| | | CCGCATACACTATTCTCAGAATGACTTGGTTGAATACTCACCAG |
| | | TCACAGAAAAGCATCTTACGGATGGCATGACAGTAAGAGAATTA |
| | | TGCAGTGCTGCCATAACCATGAGTGATAACACTGCGGCCAACTT |
| | | ACTTCTGACAACTATCGGAGGACCGAAGGAGCTAACCGCTTTTT |
| | | TGCACAACATGGGGGATCATGTAACTCGCCTTGATCGTTGGGAA |
| | | CCGGAGCTGAATGAAGCCATACCAAACGACGAGCGTGACACCA |
| | | CGATGCCTGTAGCAATGGCAACAACGTTGCGAAAACTATTAACT |
| | | GGCGAACTACTTACTCTAGCTTCCCGGCAACAACTAATAGACTG |
| | | GATGGAGGCGGATAAAGTTGCAGGACCACTTCTGCGCTCGGCAC |
| | | TTCCGGCTGGCTGGTTTATTGCTGATAAATCAGGAGCCGGTGAG |
| | | CGTGGGTCACGCGGTATCATTGCAGCACTGGGGCCGGATGGTAA |
| | | GCCCTCCCGTATCGTAGTTATCTACACTACGGGGAGTCAGGCAA |
| | | CTATGGATGAACGAAATAGACAGATCGCTGAGATAGGTGCCTCA |
| | | CTGATTAAGCATTGGTAAGGATAAATTTCTGGTAAGGAGGACAC |
| | | GTATGGAAGTGGGCAAGTTGGGGAAGCCGTATCCGTTGCTGAAT |
| | | CTGGCATATGTGGGAGTATAAGACGCGCAGCGTCGCATCAGGCA |
| | | TTTTTTTCTGCGCCAATGCAAAAAGGCCATCCGTCAGGATGGCC |
| | | TTTCGGCATAACTAGTGAGGCTCCGGTGCCCGTCAGTGGGCAGA |
| | | GCGCACATCGCCCACAGTCCCCGAGAAGTTGGGGGGAGGGGTC |
| | | GGCAATTGAACCGGTGCCTAGAGAAGGTGGCGCGGGGTAAACT |
| | | GGGAAAGTGATGTCGTGTACTGGCTCCGCCTTTTTCCCGAGGGT |
| | | GGGGGAGAACCGTATATAAGTGCAGTAGTCGCCGTGAACGTTCT |
| | | TTTTCGCAACGGGTTTGCCGCCAGAACACAGGTAAGTGCCGTGT |
| | | GTGGTTCCCGCGGGCCTGGCCTCTTTACGGGTTATGGCCCTTGCG |
| | | TGCCTTGAATTACTTCCACCTGGCTGCAGTACGTGATTCTTGATC |
| | | CCGAGCTTCGGGTTGGAAGTGGGTGGGAGAGTTCGAGGCCTTGC |
| | | GCTTAAGGAGCCCCTTCGCCTCGTGCTTGAGTTGAGGCCTGGCC |
| | | TGGGCGCTGGGGCCGCCGCGTGCGAATCTGGTGGCACCTTCGCG |
| | | CCTGTCTCGCTGCTTTCGATAAGTCTCTAGCCATTTAAAATTTTT |
| | | GATGACCTGCTGCGACGCTTTTTTTCTGGCAAGATAGTCTTGTAA |
| | | ATGCGGGCCAAGACGATCTGCACACTGGTATTTCGGTTTTTGGG |
| | | GCCGCGGGCGGCGACGGGGCCCGTGCGTCCCAGCGCACATGTTC |
| | | GGCGAGGCGGGGCCTGCGAGCGCGGCCACCGAGAATCGGACGG |
| | | GGGTAGTCTCAAGCTGGCCGGCCTGCTCTGGTGCCTGGCCTCGC |
| | | GCCGCCGTGTATCGCCCCGCCCTGGGCGGCAAGGCTGGCCCGGT |
| | | CGGCACCAGTTGCGTGAGCGGAAAGATGGCCGCTTCCCGGCCCT |
| | | GCTGCAGGGAGCTCAAAATGGAGGACGCGGCGCTCGGGAGAGC |
| | | GGGCGGGTGAGTCACCCACACAAAGGAAAAGGGCCTTTCCGTC |
| | | CTCAGCCGTCGCTTCATGTGACTCCACGGAGTACCGGGCGCCGT |
| | | CCAGGCACCTCGATTAGTTCTCGAGCTTTTGGAGTACGTCGTCTT |
| | | TAGGTTGGGGGGAGGGGTTTTATGCGATGGAGTTTCCCCACACT |
| | | GAGTGGGTGGAGACTGAAGTTAGGCCAGCTTGGCACTTGATGTA |
| | | ATTCTCCTTGGAATTTGCCCTTTTTGAGTTTGGATCTTGGTTCATT |
| | | CTCAAGCCTCAGACAGTGGTTCAAAGTTTTTTTCTTCCATTTCAG |
| | | GTGTCGTGAAA |
| 8 | P-NR-025 | AGCTAGCTTTAATACGACTCACTATAAGGAAATAAGAGAGAAA |
| | plasmid | AGAAGAGTAAGAAGAAATATAAGAGCCACCATGAAATCCTTGA |
| | | GAGTTTTACTAGTGATCCTGTGGCTTCAGTTGAGCTGGGTTTGGA |
| | | GCCAACAGAAGGAGGTGGAGCAGAATTCTGGACCCCTCAGTGTT |
| | | CCAGAGGGAGCCATTGCCTCTCTCAACTGCACTTACAGTGACCG |
| | | AGGTTCCCAGTCCTTCTTCTGGTACAGACAATATTCTGGGAAAA |
| | | GCCCTGAGTTGATAATGTCCATATACTCCAATGGTGACAAAGAA |
| | | GATGGAAGGTTTACAGCACAGCTCAATAAAGCCAGCCAGTATGT |
| | | TTCTCTGCTCATCAGAGACTCCCAGCCCAGTGATTCAGCCACCTA |
| | | CCTCTGTGCCGTGAACGTCCCCTTGAGTTACCAACTCACTTTCGG |
| | | GAAGGGGACCAAACTCTCGGTCATACCAAATGGAGGATCTGGT |
| | | GGAAGAACTGTTGCGGCGCCATCAGTGTTCATCTTTCCCCCTAG |
| | | CGACGAGCAGCTGAAGAGTGGCACAGCTTCCGTGGTCTGCCTGC |
| | | TGAACAATTTCTACCCCCGGGAAGCCAAGGTGCAGTGGAAAGTC |
| | | GATAACGCTCTGCAGTCTGGAAATAGTCAGGAGTCAGTGACTGA |
| | | ACAGGACAGCAAGGATTCCACCTATTCTCTGAGCTCCACCCTGA |
| | | CACTGTCTAAAGCAGACTACGAGAAGCACAAAGTCTATGCCTGT |
| | | GAAGTCACTCACCAGGGTCTGTCTTCACCAGTCACCAAATCCTT |
| | | CAATAGGGGGGAATGCGGCAGTGGTGAGTTTGATGCTCCAAGCC |
| | | CTCTCCCAGAGACTACAGAGAACGTGGTGTGTGCCCTGGGCCTG |
| | | ACTGTGGGTCTGGTGGGCATCATTATTGGGACCATCTTCATCATC |
| | | AGGAGTAAGAGGAGCAGGCTCCTGCACAGTGACTACATGAACA |
| | | TGACTCCCCGCCGCCCCGGGCCCACCCGCAAGCATTACCAGCCC |
| | | TATGCCCCACCACGCGACTTCGCAGCCTATCGCTCCAGAGTGAA |
| | | GTTCAGCAGGAGCGCAGACGCCCCCGCGTACCAGCAGGGCCAG |
| | | AACCAGCTCTATAACGAGCTCAATCTAGGACGAAGAGAGGAGT |
| | | ACGATGTTTTGGACAAGAGACGTGGCCGGGACCCTGAGATGGG |
| | | GGGAAAGCCGCAGAGAAGGAAGAACCCTCAGGAAGGCCTGTAC |
| | | AATGAACTGCAGAAAGATAAGATGGCGGAGGCCTACAGTGAGA |
| | | TTGGGATGAAAGGCGAGCGCCGGAGGGGCAAGGGGCACGATGG |
| | | CCTTTACCAGGGTCTCAGTACAGCCACCAAGGACACCTACGACG |
| | | CCCTTCACATGCAGGCCCTGCCCCCTCGCTGATTAATTAAGCTGC |
| | | CTTCTGCGGGGCTTGCCTTCTGGCCATGCCCTTCTTCTCTCCCTTG |
| | | CACCTGTACCTCTTGGTCTTTGAATAAAGCCTGAGTAGGAAGAA |
| | | AAAAAAAAAAAAAAAAAAAAAGCAGGTGGCGGCCGCAGGTAA |
| | | GCCAGCCCAGGCCTCGCCCTCCAGCTCAAGGCGGGACAGGTGCC |
| | | CTAGAGTAGCCTGCATCCAGGGACAGGCCCCAGCCGGGTGCTGA |
| | | CACGTCCACCTCCATCTCTTCCTCAGGTCTGCCCGGGTGGCATCC |
| | | CTGTGACCCCTCCCCAGTGCCTCTCCTGGTCGTGGAAGGTGCTAC |
| | | TCCAGTGCCCACCAGCCTTGTCCTAATAAAATTAAGTTGCATCAT |
| | | TTTGTTTGACTAGGTGTCCTTGTATAATATTATGGGGTGGAGGCG |
| | | GGTGGTATGGAGCAAGGGGCCCAAGTTAACTTGTTTATTGCAGC |
| | | TTATAATGGTTACAAATAAAGCAATAGCATCACAAATTTCACAA |
| | | ATAAAGCATTTTTTTCACTGCATTCTAGTTGTGGTTTGTCCAAAC |
| | | TCATCAATGTATCTTATCATGTCTGGATCCGCTTCAGGCACCGGG |
| | | CTTGCGGGTCATGCACCAGGTGCGCGGTCCTTCGGGCACCTCGA |
| | | CGTCGGCGGTGACGGTGAAGCCGAGCCGCTCGTAGAAGGGGAG |
| | | GTTGCGGGGCGCGGAGGTCTCCAGGAAGGCGGGCACCCCGGCG |
| | | CGCTCGGCCGCCTCCACTCCGGGGAGCACGACGGCGCTGCCCAG |
| | | ACCCTTGCCCTGGTGGTCGGGCGAGACGCCGACGGTGGCCAGGA |
| | | ACCACGCGGGCTCCTTGGGCCGGTGCGGCGCCAGGAGGCCTTCC |
| | | ATCTGTTGCTGCGCGGCCAGCCTGGAACCGCTCAACTCGGCCAT |
| | | GCGCGGGCCGATCTCGGCGAACACCGCCCCCGCTTCGACGCTCT |
| | | CCGGCGTGGTCCAGACCGCCACCGCGGCGCCGTCGTCCGCGACC |
| | | CACACCTTGCCGATGTCGAGCCCGACGCGCGTGAGGAAGAGTTC |
| | | TTGCAGCTCGGTGACCCGCTCGATGTGGCGGTCCGGGTCGACGG |
| | | TGTGGCGCGTGGCGGGGTAGTCGGCGAACGCGGCGGCGAGGGT |
| | | GCGTACGGCCCGGGGGACGTCGTCGCGGGTGGCGAGGCGCACC |
| | | GTGGGCTTGTACTCGGTCATGGTGGCCTGCAGAGTCGCTCTGTGT |
| | | TCGAGGCCACACGCGTCACCTTAATATGCGAAGTGGACCTGGGA |
| | | CCGCGCCGCCCCGACTGCATCTGCGTGTTTTCGCCAATGACAAG |
| | | ACGCTGGGCGGGGTTTGTGTCATCATAGAACTAAAGACATGCAA |
| | | ATATATTTCTTCCGGGGACACCGCCAGCAAACGCGAGCAACGGG |
| | | CCACGGGGATGAAGCAGCTGCGCCACTCCCTGAAGATCCATCGT |
| | | CTCCTAACAAGTTACATCACTCCTGCCCTTCCTCACCCTCATCTC |
| | | CATCACCTCCTTCATCTCCGTCATCTCCGTCATCACCCTCCGCGG |
| | | CAGCCCCTTCCACCATAGGTGGAAACCAGGGAGGCAAATCTACT |
| | | CCATCGTCAAAGCTGCACACAGTCACCCTGATATTGCAGGTAGG |
| | | AGCGGGCTTTGTCATAACAAGGTCCTTAATCGCATCCTTCAAAA |
| | | CCTCAGCAAATATATGAGTTTGTAAAAAGACCATGAAATAACAG |
| | | ACAATGGACTCCCTTAGCGGGCCAGGTTGTGGGCCGGGTCCAGG |
| | | GGCCATTCCAAAGGGGAGACGACTCAATGGTGTAAGACGACAT |
| | | TGTGGAATAGCAAGGGCAGTTCCTCGCCTTAGGTTGTAAAGGGA |
| | | GGTCTTACTACCTCCATATACGAACACACCGGCGACCCAAGTTC |
| | | CTTCGTCGGTAGTCCTTTCTACGTGACTCCTAGCCAGGAGAGCTC |
| | | TTAAACCTTCTGCAATGTTCTCAAATTTCGGGTTGGAACCTCCTT |
| | | GACCACGATGCTTTCCAAACCACCCTCCTTTTTTGCGCCTGCCTC |
| | | CATCACCCTGACCCCCGCTGCGCGGGGGCACGTCAGGCTCACCA |
| | | TCTGGGCCGCCTTCTTGGTGGTATTCAAAATAATCGGCTTCCCCT |
| | | ACAGGGTGGAAAAATGGCCTTCTACCTGGAGGGGGCCTGCGCG |
| | | GTGGAGACCCGGATGATGATGACTGACTACTGGGACTCCTGGGC |
| | | CTCTTTTCTCCACGTCCACGACCTCTCCCCCTGGCTCTTTCACGA |
| | | CTTCCCCCCCTGGCTCTTTCACGTCCTCTACCCCGGCGGCCTCCA |
| | | CTACCTCCTCGACCCCGGCCTCCACTACCTCCTCGACCCCGGCCT |
| | | CCACTGCCTCCTCGACCCCGGCCTCCACCTCCTGCTCCTGCCCCT |
| | | CCCGCTCCTGCTCCTGCTCCTGTTCCACCGTGGGTCCCTTTGCAG |
| | | CCAATGCAACTTGGACGTTTTTGGGGTCTCCGGACACCATCTCTA |
| | | TGTCTTGGCCCTGATCCTGAGCCGCCCGGGGCTCCTGGTCTTCCG |
| | | CCTCCTCGTCCTCGTCCTCTTCCCCGTCCTCGTCCATGTGCCATG |
| | | ATGGCGGCCTGCAGCTGTGTTCGAGGCCGCGCGTGTCACCTTAA |
| | | TATGCGAAGTGGACCTGGGACCGCGCCGCCCCGACTGCATCTGC |
| | | GTGTTCGAGTTCGCCAATGACAAGACGCTGGGCGGGGAGATCCC |
| | | CCTTATTAACCCTAAACGGGTAGCATATGCTTCCCGGGTAGTAG |
| | | TATATACTATCCAGACTAACCCTAATTCAATAGCATATGTTACCC |
| | | AACGGGAAGCATATGCTATCGAATTAGGGTTAGTAAAAGGGTCC |
| | | TAAGGAACAGCGATCTGGATAGCATATGCTATCCTAATCTATAT |
| | | CTGGGTAGCATATGCTATCCTAATCTATATCTGGGTAGCATAGG |
| | | CTATCCTAATCTATATCTGGGTAGCATATGCTATCCTAATCTATA |
| | | TCTGGGTAGTATATGCTATCCTAATTTATATCTGGGTAGCATAGG |
| | | CTATCCTAATCTATATCTGGGTAGCATATGCTATCCTAATCTATA |
| | | TCTGGGTAGTATATGCTATCCTAATCTGTATCCGGGTAGCATATG |
| | | CTATCCTCATGCATATACAGTCAGCATATGATACCCAGTAGTAG |
| | | AGTGGGAGTGCTATCCTTTGCATATGCCGCCACCTCCCAAGGAG |
| | | ATCTGTCGACATCGATGGGCGCGGGTGTACACTCCGCCCATCCC |
| | | GCCCCTAACTCCGCCCAGTTCCGCCCATTCTCCGCCTCATGGCTG |
| | | ACTAATTTTTTTTATTTATGCAGAGGCCGAGGCCGCCTCGGCCTC |
| | | TGAGCTATTCCAGAAGTAGTGAGGAGGCTTTTTTGGAGGCCTAG |
| | | GCTTTTGCAAAAAGCTAATTCGGCGTAATCTGCTGCTTGCAAAC |
| | | AAAAAAACCACCGCTACCAGCGGTGGTTTGTTTGCCGGATCAAG |
| | | AGCTACCAACTCTTTTTCCGAAGGTAACTGGCTTCAGCAGAGCG |
| | | CAGATACCAAATACTGTCCTTCTAGTGTAGCCGTAGTTAGGCCA |
| | | CCACTTCAAGAACTCTGTAGCACCGCCTACATACCTCGCTCTGCT |
| | | GAAGCCAGTTACCAGTGGCTGCTGCCAGTGGCGATAAGTCGTGT |
| | | CTTACCGGGTTGGACTCAAGAGATAGTTACCGGATAAGGCGCAG |
| | | CGGTCGGGCTGAACGGGGGGTTCGTGCACACAGCCCAGCTTGGA |
| | | GCGAACGACCTACACCGAACTGAGATACCTACAGCGTGAGCTAT |
| | | GAGAAAGCGCCACGCTTCCCGAAGGGAGAAAGGCGGACAGGTA |
| | | TCCGGTAAGCGGCAGGGTCGGAACAGGAGAGCGCACGAGGGAG |
| | | CTTCCAGGGGGAAACGCCTGGTATCTTTATAGTCCTGTCGGGTTT |
| | | CGCCACCTCTGACTTGAGCGTCGATTTTTGTGATGCTCGTCAGGG |
| | | GGGCGGAGCCTATGGAAAAACGCCAGCAACGCAAGCTAGAGTT |
| | | TAAACTTGACAGATGAGACAATAACCCTGATAAATGCTTCAATA |
| | | ATATTGAAAAAGGAAAAGTATGAGTATTCAACATTTCCGTGTCG |
| | | CCCTTATTCCCTTTTTTGCGGCATTTTGCCTTCCTGTTTTTGCTCA |
| | | CCCAGAAACGCTGGTGAAAGTAAAAGATGCAGAAGATCACTTG |
| | | GGTGCGCGAGTGGGTTACATCGAACTGGATCTCAACAGCGGTAA |
| | | GATCCTTGAGAGTTTTCGCCCCGAAGAACGTTTCCCAATGATGA |
| | | GCACTTTTAAAGTTCTGCTATGTGGCGCGGTATTATCCCGTATTG |
| | | ATGCCGGGCAAGAGCAACTCGGTCGCCGCATACACTATTCTCAG |
| | | AATGACTTGGTTGAATACTCACCAGTCACAGAAAAGCATCTTAC |
| | | GGATGGCATGACAGTAAGAGAATTATGCAGTGCTGCCATAACCA |
| | | TGAGTGATAACACTGCGGCCAACTTACTTCTGACAACTATCGGA |
| | | GGACCGAAGGAGCTAACCGCTTTTTTGCACAACATGGGGGATCA |
| | | TGTAACTCGCCTTGATCGTTGGGAACCGGAGCTGAATGAAGCCA |
| | | TACCAAACGACGAGCGTGACACCACGATGCCTGTAGCAATGGC |
| | | AACAACGTTGCGAAAACTATTAACTGGCGAACTACTTACTCTAG |
| | | CTTCCCGGCAACAACTAATAGACTGGATGGAGGCGGATAAAGTT |
| | | GCAGGACCACTTCTGCGCTCGGCACTTCCGGCTGGCTGGTTTATT |
| | | GCTGATAAATCAGGAGCCGGTGAGCGTGGGTCACGCGGTATCAT |
| | | TGCAGCACTGGGGCCGGATGGTAAGCCCTCCCGTATCGTAGTTA |
| | | TCTACACTACGGGGAGTCAGGCAACTATGGATGAACGAAATAG |
| | | ACAGATCGCTGAGATAGGTGCCTCACTGATTAAGCATTGGTAAG |
| | | GATAAATTTCTGGTAAGGAGGACACGTATGGAAGTGGGCAAGTT |
| | | GGGGAAGCCGTATCCGTTGCTGAATCTGGCATATGTGGGAGTAT |
| | | AAGACGCGCAGCGTCGCATCAGGCATTTTTTTCTGCGCCAATGC |
| | | AAAAAGGCCATCCGTCAGGATGGCCTTTCGGCATAACTAGTGAG |
| | | GCTCCGGTGCCCGTCAGTGGGCAGAGCGCACATCGCCCACAGTC |
| | | CCCGAGAAGTTGGGGGGAGGGGTCGGCAATTGAACCGGTGCCT |
| | | AGAGAAGGTGGCGCGGGGTAAACTGGGAAAGTGATGTCGTGTA |
| | | CTGGCTCCGCCTTTTTCCCGAGGGTGGGGGAGAACCGTATATAA |
| | | GTGCAGTAGTCGCCGTGAACGTTCTTTTTCGCAACGGGTTTGCCG |
| | | CCAGAACACAGGTAAGTGCCGTGTGTGGTTCCCGCGGGCCTGGC |
| | | CTCTTTACGGGTTATGGCCCTTGCGTGCCTTGAATTACTTCCACC |
| | | TGGCTGCAGTACGTGATTCTTGATCCCGAGCTTCGGGTTGGAAG |
| | | TGGGTGGGAGAGTTCGAGGCCTTGCGCTTAAGGAGCCCCTTCGC |
| | | CTCGTGCTTGAGTTGAGGCCTGGCCTGGGCGCTGGGGCCGCCGC |
| | | GTGCGAATCTGGTGGCACCTTCGCGCCTGTCTCGCTGCTTTCGAT |
| | | AAGTCTCTAGCCATTTAAAATTTTTGATGACCTGCTGCGACGCTT |
| | | TTTTTCTGGCAAGATAGTCTTGTAAATGCGGGCCAAGACGATCT |
| | | GCACACTGGTATTTCGGTTTTTGGGGCCGCGGGCGGCGACGGGG |
| | | CCCGTGCGTCCCAGCGCACATGTTCGGCGAGGCGGGGCCTGCGA |
| | | GCGCGGCCACCGAGAATCGGACGGGGGTAGTCTCAAGCTGGCC |
| | | GGCCTGCTCTGGTGCCTGGCCTCGCGCCGCCGTGTATCGCCCCG |
| | | CCCTGGGCGGCAAGGCTGGCCCGGTCGGCACCAGTTGCGTGAGC |
| | | GGAAAGATGGCCGCTTCCCGGCCCTGCTGCAGGGAGCTCAAAAT |
| | | GGAGGACGCGGCGCTCGGGAGAGCGGGCGGGTGAGTCACCCAC |
| | | ACAAAGGAAAAGGGCCTTTCCGTCCTCAGCCGTCGCTTCATGTG |
| | | ACTCCACGGAGTACCGGGCGCCGTCCAGGCACCTCGATTAGTTC |
| | | TCGAGCTTTTGGAGTACGTCGTCTTTAGGTTGGGGGGAGGGGTT |
| | | TTATGCGATGGAGTTTCCCCACACTGAGTGGGTGGAGACTGAAG |
| | | TTAGGCCAGCTTGGCACTTGATGTAATTCTCCTTGGAATTTGCCC |
| | | TTTTTGAGTTTGGATCTTGGTTCATTCTCAAGCCTCAGACAGTGG |
| | | TTCAAAGTTTTTTTCTTCCATTTCAGGTGTCGTGAAA |
| 9 | P-NR-022 | AGCTAGCTTTAATACGACTCACTATAAGGAAATAAGAGAGAAA |
| | plasmid | AGAAGAGTAAGAAGAAATATAAGAGCCACCATGGGCTAGTACA |
| | | GATATCTGGTGGAGCTTCTACAAAAGGGCCAAGCGTGTTCCCAC |
| | | TGGCACCCAGCTCCAAGTCAACCAGCGGAGGAACAGCCGCTCTG |
| | | GGATGCCTGGTGAAAGACTACTTCCCAGAGCCCGTGACCGTCTC |
| | | CTGGAACTCTGGGGCCCTGACAAGCGGTGTGCACACTTTTCCTG |
| | | CTGTCCTGCAGTCTAGTGGGCTGTACTCCCTGTCAAGCGTGGTCA |
| | | CTGTGCCATCCTCTAGTCTGGGTACTCAGACCTATATCTGCAACG |
| | | TGAATCACAAGCCTAGCAATACCAAAGTGGACAAGAAAGTCGA |
| | | ACCAAAGTCCTGTGGCAGTGGTAGAGCACGGTCTGAATCTGCAC |
| | | AGAGCAAGATGCTGAGTGGAGTCGGGGGCTTTGTGCTGGGCCTG |
| | | CTCTTCCTTGGGGCCGGGCTGTTCATCAGGAGTAAGAGGAGCAG |
| | | GCTCCTGCACAGTGACTACATGAACATGACTCCCCGCCGCCCCG |
| | | GGCCCACCCGCAAGCATTACCAGCCCTATGCCCCACCACGCGAC |
| | | TTCGCAGCCTATCGCTCCAGAGTGAAGTTCAGCAGGAGCGCAGA |
| | | CGCCCCCGCGTACCAGCAGGGCCAGAACCAGCTCTATAACGAGC |
| | | TCAATCTAGGACGAAGAGAGGAGTACGATGTTTTGGACAAGAG |
| | | ACGTGGCCGGGACCCTGAGATGGGGGGAAAGCCGCAGAGAAGG |
| | | AAGAACCCTCAGGAAGGCCTGTACAATGAACTGCAGAAAGATA |
| | | AGATGGCGGAGGCCTACAGTGAGATTGGGATGAAAGGCGAGCG |
| | | CCGGAGGGGCAAGGGGCACGATGGCCTTTACCAGGGTCTCAGT |
| | | ACAGCCACCAAGGACACCTACGACGCCCTTCACATGCAGGCCCT |
| | | GCCCCCTCGCTGATTAATTAAGCTGCCTTCTGCGGGGCTTGCCTT |
| | | CTGGCCATGCCCTTCTTCTCTCCCTTGCACCTGTACCTCTTGGTCT |
| | | TTGAATAAAGCCTGAGTAGGAAGAAAAAAAAAAAAAAAAAAAA |
| | | AAAGCAGGTGGCGGCCGCAGGTAAGCCAGCCCAGGCCTCGCCC |
| | | TCCAGCTCAAGGCGGGACAGGTGCCCTAGAGTAGCCTGCATCCA |
| | | GGGACAGGCCCCAGCCGGGTGCTGACACGTCCACCTCCATCTCT |
| | | TCCTCAGGTCTGCCCGGGTGGCATCCCTGTGACCCCTCCCCAGTG |
| | | CCTCTCCTGGTCGTGGAAGGTGCTACTCCAGTGCCCACCAGCCTT |
| | | GTCCTAATAAAATTAAGTTGCATCATTTTGTTTGACTAGGTGTCC |
| | | TTGTATAATATTATGGGGTGGAGGCGGGTGGTATGGAGCAAGGG |
| | | GCCCAAGTTAACTTGTTTATTGCAGCTTATAATGGTTACAAATAA |
| | | AGCAATAGCATCACAAATTTCACAAATAAAGCATTTTTTTCACT |
| | | GCATTCTAGTTGTGGTTTGTCCAAACTCATCAATGTATCTTATCA |
| | | TGTCTGGATCCGCTTCAGGCACCGGGCTTGCGGGTCATGCACCA |
| | | GGTGCGCGGTCCTTCGGGCACCTCGACGTCGGCGGTGACGGTGA |
| | | AGCCGAGCCGCTCGTAGAAGGGGAGGTTGCGGGGCGCGGAGGT |
| | | CTCCAGGAAGGCGGGCACCCCGGCGCGCTCGGCCGCCTCCACTC |
| | | CGGGGAGCACGACGGCGCTGCCCAGACCCTTGCCCTGGTGGTCG |
| | | GGCGAGACGCCGACGGTGGCCAGGAACCACGCGGGCTCCTTGG |
| | | GCCGGTGCGGCGCCAGGAGGCCTTCCATCTGTTGCTGCGCGGCC |
| | | AGCCTGGAACCGCTCAACTCGGCCATGCGCGGGCCGATCTCGGC |
| | | GAACACCGCCCCCGCTTCGACGCTCTCCGGCGTGGTCCAGACCG |
| | | CCACCGCGGCGCCGTCGTCCGCGACCCACACCTTGCCGATGTCG |
| | | AGCCCGACGCGCGTGAGGAAGAGTTCTTGCAGCTCGGTGACCCG |
| | | CTCGATGTGGCGGTCCGGGTCGACGGTGTGGCGCGTGGCGGGGT |
| | | AGTCGGCGAACGCGGCGGCGAGGGTGCGTACGGCCCGGGGGAC |
| | | GTCGTCGCGGGTGGCGAGGCGCACCGTGGGCTTGTACTCGGTCA |
| | | TGGTGGCCTGCAGAGTCGCTCTGTGTTCGAGGCCACACGCGTCA |
| | | CCTTAATATGCGAAGTGGACCTGGGACCGCGCCGCCCCGACTGC |
| | | ATCTGCGTGTTTTCGCCAATGACAAGACGCTGGGCGGGGTTTGT |
| | | GTCATCATAGAACTAAAGACATGCAAATATATTTCTTCCGGGGA |
| | | CACCGCCAGCAAACGCGAGCAACGGGCCACGGGGATGAAGCAG |
| | | CTGCGCCACTCCCTGAAGATCCATCGTCTCCTAACAAGTTACATC |
| | | ACTCCTGCCCTTCCTCACCCTCATCTCCATCACCTCCTTCATCTCC |
| | | GTCATCTCCGTCATCACCCTCCGCGGCAGCCCCTTCCACCATAGG |
| | | TGGAAACCAGGGAGGCAAATCTACTCCATCGTCAAAGCTGCACA |
| | | CAGTCACCCTGATATTGCAGGTAGGAGCGGGCTTTGTCATAACA |
| | | AGGTCCTTAATCGCATCCTTCAAAACCTCAGCAAATATATGAGT |
| | | TTGTAAAAAGACCATGAAATAACAGACAATGGACTCCCTTAGCG |
| | | GGCCAGGTTGTGGGCCGGGTCCAGGGGCCATTCCAAAGGGGAG |
| | | ACGACTCAATGGTGTAAGACGACATTGTGGAATAGCAAGGGCA |
| | | GTTCCTCGCCTTAGGTTGTAAAGGGAGGTCTTACTACCTCCATAT |
| | | ACGAACACACCGGCGACCCAAGTTCCTTCGTCGGTAGTCCTTTC |
| | | TACGTGACTCCTAGCCAGGAGAGCTCTTAAACCTTCTGCAATGT |
| | | TCTCAAATTTCGGGTTGGAACCTCCTTGACCACGATGCTTTCCAA |
| | | ACCACCCTCCTTTTTTGCGCCTGCCTCCATCACCCTGACCCCCGC |
| | | TGCGCGGGGGCACGTCAGGCTCACCATCTGGGCCGCCTTCTTGG |
| | | TGGTATTCAAAATAATCGGCTTCCCCTACAGGGTGGAAAAATGG |
| | | CCTTCTACCTGGAGGGGGCCTGCGCGGTGGAGACCCGGATGATG |
| | | ATGACTGACTACTGGGACTCCTGGGCCTCTTTTCTCCACGTCCAC |
| | | GACCTCTCCCCCTGGCTCTTTCACGACTTCCCCCCCTGGCTCTTT |
| | | CACGTCCTCTACCCCGGCGGCCTCCACTACCTCCTCGACCCCGGC |
| | | CTCCACTACCTCCTCGACCCCGGCCTCCACTGCCTCCTCGACCCC |
| | | GGCCTCCACCTCCTGCTCCTGCCCCTCCCGCTCCTGCTCCTGCTC |
| | | CTGTTCCACCGTGGGTCCCTTTGCAGCCAATGCAACTTGGACGTT |
| | | TTTGGGGTCTCCGGACACCATCTCTATGTCTTGGCCCTGATCCTG |
| | | AGCCGCCCGGGGCTCCTGGTCTTCCGCCTCCTCGTCCTCGTCCTC |
| | | TTCCCCGTCCTCGTCCATGTGCCATGATGGCGGCCTGCAGCTGTG |
| | | TTCGAGGCCGCGCGTGTCACCTTAATATGCGAAGTGGACCTGGG |
| | | ACCGCGCCGCCCCGACTGCATCTGCGTGTTCGAGTTCGCCAATG |
| | | ACAAGACGCTGGGCGGGGAGATCCCCCTTATTAACCCTAAACGG |
| | | GTAGCATATGCTTCCCGGGTAGTAGTATATACTATCCAGACTAA |
| | | CCCTAATTCAATAGCATATGTTACCCAACGGGAAGCATATGCTA |
| | | TCGAATTAGGGTTAGTAAAAGGGTCCTAAGGAACAGCGATCTGG |
| | | ATAGCATATGCTATCCTAATCTATATCTGGGTAGCATATGCTATC |
| | | CTAATCTATATCTGGGTAGCATAGGCTATCCTAATCTATATCTGG |
| | | GTAGCATATGCTATCCTAATCTATATCTGGGTAGTATATGCTATC |
| | | CTAATTTATATCTGGGTAGCATAGGCTATCCTAATCTATATCTGG |
| | | GTAGCATATGCTATCCTAATCTATATCTGGGTAGTATATGCTATC |
| | | CTAATCTGTATCCGGGTAGCATATGCTATCCTCATGCATATACAG |
| | | TCAGCATATGATACCCAGTAGTAGAGTGGGAGTGCTATCCTTTG |
| | | CATATGCCGCCACCTCCCAAGGAGATCTGTCGACATCGATGGGC |
| | | GCGGGTGTACACTCCGCCCATCCCGCCCCTAACTCCGCCCAGTT |
| | | CCGCCCATTCTCCGCCTCATGGCTGACTAATTTTTTTTATTTATGC |
| | | AGAGGCCGAGGCCGCCTCGGCCTCTGAGCTATTCCAGAAGTAGT |
| | | GAGGAGGCTTTTTTGGAGGCCTAGGCTTTTGCAAAAAGCTAATT |
| | | CGGCGTAATCTGCTGCTTGCAAACAAAAAAACCACCGCTACCAG |
| | | CGGTGGTTTGTTTGCCGGATCAAGAGCTACCAACTCTTTTTCCGA |
| | | AGGTAACTGGCTTCAGCAGAGCGCAGATACCAAATACTGTCCTT |
| | | CTAGTGTAGCCGTAGTTAGGCCACCACTTCAAGAACTCTGTAGC |
| | | ACCGCCTACATACCTCGCTCTGCTGAAGCCAGTTACCAGTGGCT |
| | | GCTGCCAGTGGCGATAAGTCGTGTCTTACCGGGTTGGACTCAAG |
| | | AGATAGTTACCGGATAAGGCGCAGCGGTCGGGCTGAACGGGGG |
| | | GTTCGTGCACACAGCCCAGCTTGGAGCGAACGACCTACACCGAA |
| | | CTGAGATACCTACAGCGTGAGCTATGAGAAAGCGCCACGCTTCC |
| | | CGAAGGGAGAAAGGCGGACAGGTATCCGGTAAGCGGCAGGGTC |
| | | GGAACAGGAGAGCGCACGAGGGAGCTTCCAGGGGGAAACGCCT |
| | | GGTATCTTTATAGTCCTGTCGGGTTTCGCCACCTCTGACTTGAGC |
| | | GTCGATTTTTGTGATGCTCGTCAGGGGGGCGGAGCCTATGGAAA |
| | | AACGCCAGCAACGCAAGCTAGAGTTTAAACTTGACAGATGAGA |
| | | CAATAACCCTGATAAATGCTTCAATAATATTGAAAAAGGAAAAG |
| | | TATGAGTATTCAACATTTCCGTGTCGCCCTTATTCCCTTTTTTGCG |
| | | GCATTTTGCCTTCCTGTTTTTGCTCACCCAGAAACGCTGGTGAAA |
| | | GTAAAAGATGCAGAAGATCACTTGGGTGCGCGAGTGGGTTACAT |
| | | CGAACTGGATCTCAACAGCGGTAAGATCCTTGAGAGTTTTCGCC |
| | | CCGAAGAACGTTTCCCAATGATGAGCACTTTTAAAGTTCTGCTAT |
| | | GTGGCGCGGTATTATCCCGTATTGATGCCGGGCAAGAGCAACTC |
| | | GGTCGCCGCATACACTATTCTCAGAATGACTTGGTTGAATACTC |
| | | ACCAGTCACAGAAAAGCATCTTACGGATGGCATGACAGTAAGA |
| | | GAATTATGCAGTGCTGCCATAACCATGAGTGATAACACTGCGGC |
| | | CAACTTACTTCTGACAACTATCGGAGGACCGAAGGAGCTAACCG |
| | | CTTTTTTGCACAACATGGGGGATCATGTAACTCGCCTTGATCGTT |
| | | GGGAACCGGAGCTGAATGAAGCCATACCAAACGACGAGCGTGA |
| | | CACCACGATGCCTGTAGCAATGGCAACAACGTTGCGAAAACTAT |
| | | TAACTGGCGAACTACTTACTCTAGCTTCCCGGCAACAACTAATA |
| | | GACTGGATGGAGGCGGATAAAGTTGCAGGACCACTTCTGCGCTC |
| | | GGCACTTCCGGCTGGCTGGTTTATTGCTGATAAATCAGGAGCCG |
| | | GTGAGCGTGGGTCACGCGGTATCATTGCAGCACTGGGGCCGGAT |
| | | GGTAAGCCCTCCCGTATCGTAGTTATCTACACTACGGGGAGTCA |
| | | GGCAACTATGGATGAACGAAATAGACAGATCGCTGAGATAGGT |
| | | GCCTCACTGATTAAGCATTGGTAAGGATAAATTTCTGGTAAGGA |
| | | GGACACGTATGGAAGTGGGCAAGTTGGGGAAGCCGTATCCGTTG |
| | | CTGAATCTGGCATATGTGGGAGTATAAGACGCGCAGCGTCGCAT |
| | | CAGGCATTTTTTTCTGCGCCAATGCAAAAAGGCCATCCGTCAGG |
| | | ATGGCCTTTCGGCATAACTAGTGAGGCTCCGGTGCCCGTCAGTG |
| | | GGCAGAGCGCACATCGCCCACAGTCCCCGAGAAGTTGGGGGGA |
| | | GGGGTCGGCAATTGAACCGGTGCCTAGAGAAGGTGGCGCGGGG |
| | | TAAACTGGGAAAGTGATGTCGTGTACTGGCTCCGCCTTTTTCCCG |
| | | AGGGTGGGGGAGAACCGTATATAAGTGCAGTAGTCGCCGTGAA |
| | | CGTTCTTTTTCGCAACGGGTTTGCCGCCAGAACACAGGTAAGTG |
| | | CCGTGTGTGGTTCCCGCGGGCCTGGCCTCTTTACGGGTTATGGCC |
| | | CTTGCGTGCCTTGAATTACTTCCACCTGGCTGCAGTACGTGATTC |
| | | TTGATCCCGAGCTTCGGGTTGGAAGTGGGTGGGAGAGTTCGAGG |
| | | CCTTGCGCTTAAGGAGCCCCTTCGCCTCGTGCTTGAGTTGAGGCC |
| | | TGGCCTGGGCGCTGGGGCCGCCGCGTGCGAATCTGGTGGCACCT |
| | | TCGCGCCTGTCTCGCTGCTTTCGATAAGTCTCTAGCCATTTAAAA |
| | | TTTTTGATGACCTGCTGCGACGCTTTTTTTCTGGCAAGATAGTCT |
| | | TGTAAATGCGGGCCAAGACGATCTGCACACTGGTATTTCGGTTT |
| | | TTGGGGCCGCGGGCGGCGACGGGGCCCGTGCGTCCCAGCGCAC |
| | | ATGTTCGGCGAGGCGGGGCCTGCGAGCGCGGCCACCGAGAATC |
| | | GGACGGGGGTAGTCTCAAGCTGGCCGGCCTGCTCTGGTGCCTGG |
| | | CCTCGCGCCGCCGTGTATCGCCCCGCCCTGGGCGGCAAGGCTGG |
| | | CCCGGTCGGCACCAGTTGCGTGAGCGGAAAGATGGCCGCTTCCC |
| | | GGCCCTGCTGCAGGGAGCTCAAAATGGAGGACGCGGCGCTCGG |
| | | GAGAGCGGGCGGGTGAGTCACCCACACAAAGGAAAAGGGCCTT |
| | | TCCGTCCTCAGCCGTCGCTTCATGTGACTCCACGGAGTACCGGG |
| | | CGCCGTCCAGGCACCTCGATTAGTTCTCGAGCTTTTGGAGTACGT |
| | | CGTCTTTAGGTTGGGGGGAGGGGTTTTATGCGATGGAGTTTCCC |
| | | CACACTGAGTGGGTGGAGACTGAAGTTAGGCCAGCTTGGCACTT |
| | | GATGTAATTCTCCTTGGAATTTGCCCTTTTTGAGTTTGGATCTTG |
| | | GTTCATTCTCAAGCCTCAGACAGTGGTTCAAAGTTTTTTTCTTCC |
| | | ATTTCAGGTGTCGTGAAA |
| 10 | P-NR-026 | AGCTAGCTTTAATACGACTCACTATAAGGAAATAAGAGAGAAA |
| | plasmid | AGAAGAGTAAGAAGAAATATAAGAGCCACCATGCTGAGTCTTCT |
| | | GCTCCTTCTCCTGGGACTAGGCTCTGTGTTCAGTGCTGTCATCTC |
| | | TCAAAAGCCAAGCAGGGATATCTGTCAACGTGGAACCTCCCTGA |
| | | CGATCCAGTGTCAAGTCGATAGCCAAGTCACCATGATGTTCTGG |
| | | TACCGTCAGCAACCTGGACAGAGCCTGACACTGATCGCAACTGC |
| | | AAATCAGGGCTCTGAGGCCACATATGAGAGTGGATTTGTCATTG |
| | | ACAAGTTTCCCATCAGCCGCCCAAACCTAACATTCTCAACTCTG |
| | | ACTGTGAGCAACATGAGCCCTGAAGACAGCAGCATATATCTCTG |
| | | CAGCGTTGCCGGGACTATCGACGAGCAGTACTTCGGGCCGGGCA |
| | | CCAGGCTCACGGTCACAGAGGGAGGATCTGGTGGAGCTTCTACA |
| | | AAAGGGCCAAGCGTGTTCCCACTGGCACCCAGCTCCAAGTCAAC |
| | | CAGCGGAGGAACAGCCGCTCTGGGATGCCTGGTGAAAGACTAC |
| | | TTCCCAGAGCCCGTGACCGTCTCCTGGAACTCTGGGGCCCTGAC |
| | | AAGCGGTGTGCACACTTTTCCTGCTGTCCTGCAGTCTAGTGGGCT |
| | | GTACTCCCTGTCAAGCGTGGTCACTGTGCCATCCTCTAGTCTGGG |
| | | TACTCAGACCTATATCTGCAACGTGAATCACAAGCCTAGCAATA |
| | | CCAAAGTGGACAAGAAAGTCGAACCAAAGTCCTGTGGCAGTGG |
| | | TAGAGCACGGTCTGAATCTGCACAGAGCAAGATGCTGAGTGGA |
| | | GTCGGGGGCTTTGTGCTGGGCCTGCTCTTCCTTGGGGCCGGGCT |
| | | GTTCATCAGGAGTAAGAGGAGCAGGCTCCTGCACAGTGACTACA |
| | | TGAACATGACTCCCCGCCGCCCCGGGCCCACCCGCAAGCATTAC |
| | | CAGCCCTATGCCCCACCACGCGACTTCGCAGCCTATCGCTCCAG |
| | | AGTGAAGTTCAGCAGGAGCGCAGACGCCCCCGCGTACCAGCAG |
| | | GGCCAGAACCAGCTCTATAACGAGCTCAATCTAGGACGAAGAG |
| | | AGGAGTACGATGTTTTGGACAAGAGACGTGGCCGGGACCCTGA |
| | | GATGGGGGGAAAGCCGCAGAGAAGGAAGAACCCTCAGGAAGG |
| | | CCTGTACAATGAACTGCAGAAAGATAAGATGGCGGAGGCCTAC |
| | | AGTGAGATTGGGATGAAAGGCGAGCGCCGGAGGGGCAAGGGGC |
| | | ACGATGGCCTTTACCAGGGTCTCAGTACAGCCACCAAGGACACC |
| | | TACGACGCCCTTCACATGCAGGCCCTGCCCCCTCGCTGATTAATT |
| | | AAGCTGCCTTCTGCGGGGCTTGCCTTCTGGCCATGCCCTTCTTCT |
| | | CTCCCTTGCACCTGTACCTCTTGGTCTTTGAATAAAGCCTGAGTA |
| | | GGAAGAAAAAAAAAAAAAAAAAAAAAAAGCAGGTGGCGGCCG |
| | | CAGGTAAGCCAGCCCAGGCCTCGCCCTCCAGCTCAAGGCGGGAC |
| | | AGGTGCCCTAGAGTAGCCTGCATCCAGGGACAGGCCCCAGCCG |
| | | GGTGCTGACACGTCCACCTCCATCTCTTCCTCAGGTCTGCCCGGG |
| | | TGGCATCCCTGTGACCCCTCCCCAGTGCCTCTCCTGGTCGTGGAA |
| | | GGTGCTACTCCAGTGCCCACCAGCCTTGTCCTAATAAAATTAAG |
| | | TTGCATCATTTTGTTTGACTAGGTGTCCTTGTATAATATTATGGG |
| | | GTGGAGGCGGGTGGTATGGAGCAAGGGGCCCAAGTTAACTTGTT |
| | | TATTGCAGCTTATAATGGTTACAAATAAAGCAATAGCATCACAA |
| | | ATTTCACAAATAAAGCATTTTTTTCACTGCATTCTAGTTGTGGTT |
| | | TGTCCAAACTCATCAATGTATCTTATCATGTCTGGATCCGCTTCA |
| | | GGCACCGGGCTTGCGGGTCATGCACCAGGTGCGCGGTCCTTCGG |
| | | GCACCTCGACGTCGGCGGTGACGGTGAAGCCGAGCCGCTCGTAG |
| | | AAGGGGAGGTTGCGGGGCGCGGAGGTCTCCAGGAAGGCGGGCA |
| | | CCCCGGCGCGCTCGGCCGCCTCCACTCCGGGGAGCACGACGGCG |
| | | CTGCCCAGACCCTTGCCCTGGTGGTCGGGCGAGACGCCGACGGT |
| | | GGCCAGGAACCACGCGGGCTCCTTGGGCCGGTGCGGCGCCAGG |
| | | AGGCCTTCCATCTGTTGCTGCGCGGCCAGCCTGGAACCGCTCAA |
| | | CTCGGCCATGCGCGGGCCGATCTCGGCGAACACCGCCCCCGCTT |
| | | CGACGCTCTCCGGCGTGGTCCAGACCGCCACCGCGGCGCCGTCG |
| | | TCCGCGACCCACACCTTGCCGATGTCGAGCCCGACGCGCGTGAG |
| | | GAAGAGTTCTTGCAGCTCGGTGACCCGCTCGATGTGGCGGTCCG |
| | | GGTCGACGGTGTGGCGCGTGGCGGGGTAGTCGGCGAACGCGGC |
| | | GGCGAGGGTGCGTACGGCCCGGGGGACGTCGTCGCGGGTGGCG |
| | | AGGCGCACCGTGGGCTTGTACTCGGTCATGGTGGCCTGCAGAGT |
| | | CGCTCTGTGTTCGAGGCCACACGCGTCACCTTAATATGCGAAGT |
| | | GGACCTGGGACCGCGCCGCCCCGACTGCATCTGCGTGTTTTCGC |
| | | CAATGACAAGACGCTGGGGGGGTTTGTGTCATCATAGAACTAA |
| | | AGACATGCAAATATATTTCTTCCGGGGACACCGCCAGCAAACGC |
| | | GAGCAACGGGCCACGGGGATGAAGCAGCTGCGCCACTCCCTGA |
| | | AGATCCATCGTCTCCTAACAAGTTACATCACTCCTGCCCTTCCTC |
| | | ACCCTCATCTCCATCACCTCCTTCATCTCCGTCATCTCCGTCATC |
| | | ACCCTCCGCGGCAGCCCCTTCCACCATAGGTGGAAACCAGGGAG |
| | | GCAAATCTACTCCATCGTCAAAGCTGCACACAGTCACCCTGATA |
| | | TTGCAGGTAGGAGCGGGCTTTGTCATAACAAGGTCCTTAATCGC |
| | | ATCCTTCAAAACCTCAGCAAATATATGAGTTTGTAAAAAGACCA |
| | | TGAAATAACAGACAATGGACTCCCTTAGCGGGCCAGGTTGTGGG |
| | | CCGGGTCCAGGGGCCATTCCAAAGGGGAGACGACTCAATGGTG |
| | | TAAGACGACATTGTGGAATAGCAAGGGCAGTTCCTCGCCTTAGG |
| | | TTGTAAAGGGAGGTCTTACTACCTCCATATACGAACACACCGGC |
| | | GACCCAAGTTCCTTCGTCGGTAGTCCTTTCTACGTGACTCCTAGC |
| | | CAGGAGAGCTCTTAAACCTTCTGCAATGTTCTCAAATTTCGGGTT |
| | | GGAACCTCCTTGACCACGATGCTTTCCAAACCACCCTCCTTTTTT |
| | | GCGCCTGCCTCCATCACCCTGACCCCCGCTGCGCGGGGGCACGT |
| | | CAGGCTCACCATCTGGGCCGCCTTCTTGGTGGTATTCAAAATAA |
| | | TCGGCTTCCCCTACAGGGTGGAAAAATGGCCTTCTACCTGGAGG |
| | | GGGCCTGCGCGGTGGAGACCCGGATGATGATGACTGACTACTGG |
| | | GACTCCTGGGCCTCTTTTCTCCACGTCCACGACCTCTCCCCCTGG |
| | | CTCTTTCACGACTTCCCCCCCTGGCTCTTTCACGTCCTCTACCCC |
| | | GGCGGCCTCCACTACCTCCTCGACCCCGGCCTCCACTACCTCCTC |
| | | GACCCCGGCCTCCACTGCCTCCTCGACCCCGGCCTCCACCTCCTG |
| | | CTCCTGCCCCTCCCGCTCCTGCTCCTGCTCCTGTTCCACCGTGGG |
| | | TCCCTTTGCAGCCAATGCAACTTGGACGTTTTTGGGGTCTCCGGA |
| | | CACCATCTCTATGTCTTGGCCCTGATCCTGAGCCGCCCGGGGCTC |
| | | CTGGTCTTCCGCCTCCTCGTCCTCGTCCTCTTCCCCGTCCTCGTCC |
| | | ATGTGCCATGATGGCGGCCTGCAGCTGTGTTCGAGGCCGCGCGT |
| | | GTCACCTTAATATGCGAAGTGGACCTGGGACCGCGCCGCCCCGA |
| | | CTGCATCTGCGTGTTCGAGTTCGCCAATGACAAGACGCTGGGCG |
| | | GGGAGATCCCCCTTATTAACCCTAAACGGGTAGCATATGCTTCC |
| | | CGGGTAGTAGTATATACTATCCAGACTAACCCTAATTCAATAGC |
| | | ATATGTTACCCAACGGGAAGCATATGCTATCGAATTAGGGTTAG |
| | | TAAAAGGGTCCTAAGGAACAGCGATCTGGATAGCATATGCTATC |
| | | CTAATCTATATCTGGGTAGCATATGCTATCCTAATCTATATCTGG |
| | | GTAGCATAGGCTATCCTAATCTATATCTGGGTAGCATATGCTATC |
| | | CTAATCTATATCTGGGTAGTATATGCTATCCTAATTTATATCTGG |
| | | GTAGCATAGGCTATCCTAATCTATATCTGGGTAGCATATGCTATC |
| | | CTAATCTATATCTGGGTAGTATATGCTATCCTAATCTGTATCCGG |
| | | GTAGCATATGCTATCCTCATGCATATACAGTCAGCATATGATAC |
| | | CCAGTAGTAGAGTGGGAGTGCTATCCTTTGCATATGCCGCCACC |
| | | TCCCAAGGAGATCTGTCGACATCGATGGGCGCGGGTGTACACTC |
| | | CGCCCATCCCGCCCCTAACTCCGCCCAGTTCCGCCCATTCTCCGC |
| | | CTCATGGCTGACTAATTTTTTTTATTTATGCAGAGGCCGAGGCCG |
| | | CCTCGGCCTCTGAGCTATTCCAGAAGTAGTGAGGAGGCTTTTTTG |
| | | GAGGCCTAGGCTTTTGCAAAAAGCTAATTCGGCGTAATCTGCTG |
| | | CTTGCAAACAAAAAAACCACCGCTACCAGCGGTGGTTTGTTTGC |
| | | CGGATCAAGAGCTACCAACTCTTTTTCCGAAGGTAACTGGCTTC |
| | | AGCAGAGCGCAGATACCAAATACTGTCCTTCTAGTGTAGCCGTA |
| | | GTTAGGCCACCACTTCAAGAACTCTGTAGCACCGCCTACATACC |
| | | TCGCTCTGCTGAAGCCAGTTACCAGTGGCTGCTGCCAGTGGCGA |
| | | TAAGTCGTGTCTTACCGGGTTGGACTCAAGAGATAGTTACCGGA |
| | | TAAGGCGCAGCGGTCGGGCTGAACGGGGGGTTCGTGCACACAG |
| | | CCCAGCTTGGAGCGAACGACCTACACCGAACTGAGATACCTACA |
| | | GCGTGAGCTATGAGAAAGCGCCACGCTTCCCGAAGGGAGAAAG |
| | | GCGGACAGGTATCCGGTAAGCGGCAGGGTCGGAACAGGAGAGC |
| | | GCACGAGGGAGCTTCCAGGGGGAAACGCCTGGTATCTTTATAGT |
| | | CCTGTCGGGTTTCGCCACCTCTGACTTGAGCGTCGATTTTTGTGA |
| | | TGCTCGTCAGGGGGGCGGAGCCTATGGAAAAACGCCAGCAACG |
| | | CAAGCTAGAGTTTAAACTTGACAGATGAGACAATAACCCTGATA |
| | | AATGCTTCAATAATATTGAAAAAGGAAAAGTATGAGTATTCAAC |
| | | ATTTCCGTGTCGCCCTTATTCCCTTTTTTGCGGCATTTTGCCTTCC |
| | | TGTTTTTGCTCACCCAGAAACGCTGGTGAAAGTAAAAGATGCAG |
| | | AAGATCACTTGGGTGCGCGAGTGGGTTACATCGAACTGGATCTC |
| | | AACAGCGGTAAGATCCTTGAGAGTTTTCGCCCCGAAGAACGTTT |
| | | CCCAATGATGAGCACTTTTAAAGTTCTGCTATGTGGCGCGGTATT |
| | | ATCCCGTATTGATGCCGGGCAAGAGCAACTCGGTCGCCGCATAC |
| | | ACTATTCTCAGAATGACTTGGTTGAATACTCACCAGTCACAGAA |
| | | AAGCATCTTACGGATGGCATGACAGTAAGAGAATTATGCAGTGC |
| | | TGCCATAACCATGAGTGATAACACTGCGGCCAACTTACTTCTGA |
| | | CAACTATCGGAGGACCGAAGGAGCTAACCGCTTTTTTGCACAAC |
| | | ATGGGGGATCATGTAACTCGCCTTGATCGTTGGGAACCGGAGCT |
| | | GAATGAAGCCATACCAAACGACGAGCGTGACACCACGATGCCT |
| | | GTAGCAATGGCAACAACGTTGCGAAAACTATTAACTGGCGAACT |
| | | ACTTACTCTAGCTTCCCGGCAACAACTAATAGACTGGATGGAGG |
| | | CGGATAAAGTTGCAGGACCACTTCTGCGCTCGGCACTTCCGGCT |
| | | GGCTGGTTTATTGCTGATAAATCAGGAGCCGGTGAGCGTGGGTC |
| | | ACGCGGTATCATTGCAGCACTGGGGCCGGATGGTAAGCCCTCCC |
| | | GTATCGTAGTTATCTACACTACGGGGAGTCAGGCAACTATGGAT |
| | | GAACGAAATAGACAGATCGCTGAGATAGGTGCCTCACTGATTAA |
| | | GCATTGGTAAGGATAAATTTCTGGTAAGGAGGACACGTATGGAA |
| | | GTGGGCAAGTTGGGGAAGCCGTATCCGTTGCTGAATCTGGCATA |
| | | TGTGGGAGTATAAGACGCGCAGCGTCGCATCAGGCATTTTTTTC |
| | | TGCGCCAATGCAAAAAGGCCATCCGTCAGGATGGCCTTTCGGCA |
| | | TAACTAGTGAGGCTCCGGTGCCCGTCAGTGGGCAGAGCGCACAT |
| | | CGCCCACAGTCCCCGAGAAGTTGGGGGGAGGGGTCGGCAATTG |
| | | AACCGGTGCCTAGAGAAGGTGGCGCGGGGTAAACTGGGAAAGT |
| | | GATGTCGTGTACTGGCTCCGCCTTTTTCCCGAGGGTGGGGGAGA |
| | | ACCGTATATAAGTGCAGTAGTCGCCGTGAACGTTCTTTTTCGCAA |
| | | CGGGTTTGCCGCCAGAACACAGGTAAGTGCCGTGTGTGGTTCCC |
| | | GCGGGCCTGGCCTCTTTACGGGTTATGGCCCTTGCGTGCCTTGAA |
| | | TTACTTCCACCTGGCTGCAGTACGTGATTCTTGATCCCGAGCTTC |
| | | GGGTTGGAAGTGGGTGGGAGAGTTCGAGGCCTTGCGCTTAAGGA |
| | | GCCCCTTCGCCTCGTGCTTGAGTTGAGGCCTGGCCTGGGCGCTG |
| | | GGGCCGCCGCGTGCGAATCTGGTGGCACCTTCGCGCCTGTCTCG |
| | | CTGCTTTCGATAAGTCTCTAGCCATTTAAAATTTTTGATGACCTG |
| | | CTGCGACGCTTTTTTTCTGGCAAGATAGTCTTGTAAATGCGGGCC |
| | | AAGACGATCTGCACACTGGTATTTCGGTTTTTGGGGCCGCGGGC |
| | | GGCGACGGGGCCCGTGCGTCCCAGCGCACATGTTCGGCGAGGC |
| | | GGGGCCTGCGAGCGCGGCCACCGAGAATCGGACGGGGGTAGTC |
| | | TCAAGCTGGCCGGCCTGCTCTGGTGCCTGGCCTCGCGCCGCCGT |
| | | GTATCGCCCCGCCCTGGGCGGCAAGGCTGGCCCGGTCGGCACCA |
| | | GTTGCGTGAGCGGAAAGATGGCCGCTTCCCGGCCCTGCTGCAGG |
| | | GAGCTCAAAATGGAGGACGCGGCGCTCGGGAGAGCGGGCGGGT |
| | | GAGTCACCCACACAAAGGAAAAGGGCCTTTCCGTCCTCAGCCGT |
| | | CGCTTCATGTGACTCCACGGAGTACCGGGCGCCGTCCAGGCACC |
| | | TCGATTAGTTCTCGAGCTTTTGGAGTACGTCGTCTTTAGGTTGGG |
| | | GGGAGGGGTTTTATGCGATGGAGTTTCCCCACACTGAGTGGGTG |
| | | GAGACTGAAGTTAGGCCAGCTTGGCACTTGATGTAATTCTCCTT |
| | | GGAATTTGCCCTTTTTGAGTTTGGATCTTGGTTCATTCTCAAGCC |
| | | TCAGACAGTGGTTCAAAGTTTTTTTCTTCCATTTCAGGTGTCGTG |
| | | AAA |
| 11 | P-NR-023 | AGCTAGCTTTAATACGACTCACTATAAGGAAATAAGAGAGAAA |
| | plasmid | AGAAGAGTAAGAAGAAATATAAGAGCCACCATGGGCTAGTACA |
| | | GATATCTAGTACAGGTAGCTCCACTGGGCCGGGTAGCACATCAG |
| | | TTGATGAGTTGCAGGCCGAGGTGGACCAGCTTCAAGATGAGAAC |
| | | TACGCTCTGAAAACGAAAGTAGCACAGTTGCGAAAAAAGGTAG |
| | | AGAAGCTCGCGTCTGGTGGATGTGGTGGAGAGTTTGATGCTCCA |
| | | AGCCCTCTCCCAGAGACTACAGAGAACGTGGTGTGTGCCCTGGG |
| | | CCTGACTGTGGGTCTGGTGGGCATCATTATTGGGACCATCTTCAT |
| | | CATCAGGAGTAAGAGGAGCAGGCTCCTGCACAGTGACTACATG |
| | | AACATGACTCCCCGCCGCCCCGGGCCCACCCGCAAGCATTACCA |
| | | GCCCTATGCCCCACCACGCGACTTCGCAGCCTATCGCTCCAGAG |
| | | TGAAGTTCAGCAGGAGCGCAGACGCCCCCGCGTACCAGCAGGG |
| | | CCAGAACCAGCTCTATAACGAGCTCAATCTAGGACGAAGAGAG |
| | | GAGTACGATGTTTTGGACAAGAGACGTGGCCGGGACCCTGAGAT |
| | | GGGGGGAAAGCCGCAGAGAAGGAAGAACCCTCAGGAAGGCCTG |
| | | TACAATGAACTGCAGAAAGATAAGATGGCGGAGGCCTACAGTG |
| | | AGATTGGGATGAAAGGCGAGCGCCGGAGGGGCAAGGGGCACGA |
| | | TGGCCTTTACCAGGGTCTCAGTACAGCCACCAAGGACACCTACG |
| | | ACGCCCTTCACATGCAGGCCCTGCCCCCTCGCTGATTAATTAAG |
| | | CTGCCTTCTGCGGGGCTTGCCTTCTGGCCATGCCCTTCTTCTCTC |
| | | CCTTGCACCTGTACCTCTTGGTCTTTGAATAAAGCCTGAGTAGGA |
| | | AGAAAAAAAAAAAAAAAAAAAAAAAGCAGGTGGCGGCCGCAG |
| | | GTAAGCCAGCCCAGGCCTCGCCCTCCAGCTCAAGGCGGGACAG |
| | | GTGCCCTAGAGTAGCCTGCATCCAGGGACAGGCCCCAGCCGGGT |
| | | GCTGACACGTCCACCTCCATCTCTTCCTCAGGTCTGCCCGGGTGG |
| | | CATCCCTGTGACCCCTCCCCAGTGCCTCTCCTGGTCGTGGAAGGT |
| | | GCTACTCCAGTGCCCACCAGCCTTGTCCTAATAAAATTAAGTTG |
| | | CATCATTTTGTTTGACTAGGTGTCCTTGTATAATATTATGGGGTG |
| | | GAGGCGGGTGGTATGGAGCAAGGGGCCCAAGTTAACTTGTTTAT |
| | | TGCAGCTTATAATGGTTACAAATAAAGCAATAGCATCACAAATT |
| | | TCACAAATAAAGCATTTTTTTCACTGCATTCTAGTTGTGGTTTGT |
| | | CCAAACTCATCAATGTATCTTATCATGTCTGGATCCGCTTCAGGC |
| | | ACCGGGCTTGCGGGTCATGCACCAGGTGCGCGGTCCTTCGGGCA |
| | | CCTCGACGTCGGCGGTGACGGTGAAGCCGAGCCGCTCGTAGAA |
| | | GGGGAGGTTGCGGGGCGCGGAGGTCTCCAGGAAGGCGGGCACC |
| | | CCGGCGCGCTCGGCCGCCTCCACTCCGGGGAGCACGACGGCGCT |
| | | GCCCAGACCCTTGCCCTGGTGGTCGGGCGAGACGCCGACGGTGG |
| | | CCAGGAACCACGCGGGCTCCTTGGGCCGGTGCGGCGCCAGGAG |
| | | GCCTTCCATCTGTTGCTGCGCGGCCAGCCTGGAACCGCTCAACT |
| | | CGGCCATGCGCGGGCCGATCTCGGCGAACACCGCCCCCGCTTCG |
| | | ACGCTCTCCGGCGTGGTCCAGACCGCCACCGCGGCGCCGTCGTC |
| | | CGCGACCCACACCTTGCCGATGTCGAGCCCGACGCGCGTGAGGA |
| | | AGAGTTCTTGCAGCTCGGTGACCCGCTCGATGTGGCGGTCCGGG |
| | | TCGACGGTGTGGCGCGTGGCGGGGTAGTCGGCGAACGCGGCGG |
| | | CGAGGGTGCGTACGGCCCGGGGGACGTCGTCGCGGGTGGCGAG |
| | | GCGCACCGTGGGCTTGTACTCGGTCATGGTGGCCTGCAGAGTCG |
| | | CTCTGTGTTCGAGGCCACACGCGTCACCTTAATATGCGAAGTGG |
| | | ACCTGGGACCGCGCCGCCCCGACTGCATCTGCGTGTTTTCGCCA |
| | | ATGACAAGACGCTGGGCGGGGTTTGTGTCATCATAGAACTAAAG |
| | | ACATGCAAATATATTTCTTCCGGGGACACCGCCAGCAAACGCGA |
| | | GCAACGGGCCACGGGGATGAAGCAGCTGCGCCACTCCCTGAAG |
| | | ATCCATCGTCTCCTAACAAGTTACATCACTCCTGCCCTTCCTCAC |
| | | CCTCATCTCCATCACCTCCTTCATCTCCGTCATCTCCGTCATCAC |
| | | CCTCCGCGGCAGCCCCTTCCACCATAGGTGGAAACCAGGGAGGC |
| | | AAATCTACTCCATCGTCAAAGCTGCACACAGTCACCCTGATATT |
| | | GCAGGTAGGAGCGGGCTTTGTCATAACAAGGTCCTTAATCGCAT |
| | | CCTTCAAAACCTCAGCAAATATATGAGTTTGTAAAAAGACCATG |
| | | AAATAACAGACAATGGACTCCCTTAGCGGGCCAGGTTGTGGGCC |
| | | GGGTCCAGGGGCCATTCCAAAGGGGAGACGACTCAATGGTGTA |
| | | AGACGACATTGTGGAATAGCAAGGGCAGTTCCTCGCCTTAGGTT |
| | | GTAAAGGGAGGTCTTACTACCTCCATATACGAACACACCGGCGA |
| | | CCCAAGTTCCTTCGTCGGTAGTCCTTTCTACGTGACTCCTAGCCA |
| | | GGAGAGCTCTTAAACCTTCTGCAATGTTCTCAAATTTCGGGTTGG |
| | | AACCTCCTTGACCACGATGCTTTCCAAACCACCCTCCTTTTTTGC |
| | | GCCTGCCTCCATCACCCTGACCCCCGCTGCGCGGGGGCACGTCA |
| | | GGCTCACCATCTGGGCCGCCTTCTTGGTGGTATTCAAAATAATC |
| | | GGCTTCCCCTACAGGGTGGAAAAATGGCCTTCTACCTGGAGGGG |
| | | GCCTGCGCGGTGGAGACCCGGATGATGATGACTGACTACTGGGA |
| | | CTCCTGGGCCTCTTTTCTCCACGTCCACGACCTCTCCCCCTGGCT |
| | | CTTTCACGACTTCCCCCCCTGGCTCTTTCACGTCCTCTACCCCGG |
| | | CGGCCTCCACTACCTCCTCGACCCCGGCCTCCACTACCTCCTCGA |
| | | CCCCGGCCTCCACTGCCTCCTCGACCCCGGCCTCCACCTCCTGCT |
| | | CCTGCCCCTCCCGCTCCTGCTCCTGCTCCTGTTCCACCGTGGGTC |
| | | CCTTTGCAGCCAATGCAACTTGGACGTTTTTGGGGTCTCCGGAC |
| | | ACCATCTCTATGTCTTGGCCCTGATCCTGAGCCGCCCGGGGCTCC |
| | | TGGTCTTCCGCCTCCTCGTCCTCGTCCTCTTCCCCGTCCTCGTCCA |
| | | TGTGCCATGATGGCGGCCTGCAGCTGTGTTCGAGGCCGCGCGTG |
| | | TCACCTTAATATGCGAAGTGGACCTGGGACCGCGCCGCCCCGAC |
| | | TGCATCTGCGTGTTCGAGTTCGCCAATGACAAGACGCTGGGCGG |
| | | GGAGATCCCCCTTATTAACCCTAAACGGGTAGCATATGCTTCCC |
| | | GGGTAGTAGTATATACTATCCAGACTAACCCTAATTCAATAGCA |
| | | TATGTTACCCAACGGGAAGCATATGCTATCGAATTAGGGTTAGT |
| | | AAAAGGGTCCTAAGGAACAGCGATCTGGATAGCATATGCTATCC |
| | | TAATCTATATCTGGGTAGCATATGCTATCCTAATCTATATCTGGG |
| | | TAGCATAGGCTATCCTAATCTATATCTGGGTAGCATATGCTATCC |
| | | TAATCTATATCTGGGTAGTATATGCTATCCTAATTTATATCTGGG |
| | | TAGCATAGGCTATCCTAATCTATATCTGGGTAGCATATGCTATCC |
| | | TAATCTATATCTGGGTAGTATATGCTATCCTAATCTGTATCCGGG |
| | | TAGCATATGCTATCCTCATGCATATACAGTCAGCATATGATACC |
| | | CAGTAGTAGAGTGGGAGTGCTATCCTTTGCATATGCCGCCACCT |
| | | CCCAAGGAGATCTGTCGACATCGATGGGCGCGGGTGTACACTCC |
| | | GCCCATCCCGCCCCTAACTCCGCCCAGTTCCGCCCATTCTCCGCC |
| | | TCATGGCTGACTAATTTTTTTTATTTATGCAGAGGCCGAGGCCGC |
| | | CTCGGCCTCTGAGCTATTCCAGAAGTAGTGAGGAGGCTTTTTTG |
| | | GAGGCCTAGGCTTTTGCAAAAAGCTAATTCGGCGTAATCTGCTG |
| | | CTTGCAAACAAAAAAACCACCGCTACCAGCGGTGGTTTGTTTGC |
| | | CGGATCAAGAGCTACCAACTCTTTTTCCGAAGGTAACTGGCTTC |
| | | AGCAGAGCGCAGATACCAAATACTGTCCTTCTAGTGTAGCCGTA |
| | | GTTAGGCCACCACTTCAAGAACTCTGTAGCACCGCCTACATACC |
| | | TCGCTCTGCTGAAGCCAGTTACCAGTGGCTGCTGCCAGTGGCGA |
| | | TAAGTCGTGTCTTACCGGGTTGGACTCAAGAGATAGTTACCGGA |
| | | TAAGGCGCAGCGGTCGGGCTGAACGGGGGGTTCGTGCACACAG |
| | | CCCAGCTTGGAGCGAACGACCTACACCGAACTGAGATACCTACA |
| | | GCGTGAGCTATGAGAAAGCGCCACGCTTCCCGAAGGGAGAAAG |
| | | GCGGACAGGTATCCGGTAAGCGGCAGGGTCGGAACAGGAGAGC |
| | | GCACGAGGGAGCTTCCAGGGGGAAACGCCTGGTATCTTTATAGT |
| | | CCTGTCGGGTTTCGCCACCTCTGACTTGAGCGTCGATTTTTGTGA |
| | | TGCTCGTCAGGGGGGCGGAGCCTATGGAAAAACGCCAGCAACG |
| | | CAAGCTAGAGTTTAAACTTGACAGATGAGACAATAACCCTGATA |
| | | AATGCTTCAATAATATTGAAAAAGGAAAAGTATGAGTATTCAAC |
| | | ATTTCCGTGTCGCCCTTATTCCCTTTTTTGCGGCATTTTGCCTTCC |
| | | TGTTTTTGCTCACCCAGAAACGCTGGTGAAAGTAAAAGATGCAG |
| | | AAGATCACTTGGGTGCGCGAGTGGGTTACATCGAACTGGATCTC |
| | | AACAGCGGTAAGATCCTTGAGAGTTTTCGCCCCGAAGAACGTTT |
| | | CCCAATGATGAGCACTTTTAAAGTTCTGCTATGTGGCGCGGTATT |
| | | ATCCCGTATTGATGCCGGGCAAGAGCAACTCGGTCGCCGCATAC |
| | | ACTATTCTCAGAATGACTTGGTTGAATACTCACCAGTCACAGAA |
| | | AAGCATCTTACGGATGGCATGACAGTAAGAGAATTATGCAGTGC |
| | | TGCCATAACCATGAGTGATAACACTGCGGCCAACTTACTTCTGA |
| | | CAACTATCGGAGGACCGAAGGAGCTAACCGCTTTTTTGCACAAC |
| | | ATGGGGGATCATGTAACTCGCCTTGATCGTTGGGAACCGGAGCT |
| | | GAATGAAGCCATACCAAACGACGAGCGTGACACCACGATGCCT |
| | | GTAGCAATGGCAACAACGTTGCGAAAACTATTAACTGGCGAACT |
| | | ACTTACTCTAGCTTCCCGGCAACAACTAATAGACTGGATGGAGG |
| | | CGGATAAAGTTGCAGGACCACTTCTGCGCTCGGCACTTCCGGCT |
| | | GGCTGGTTTATTGCTGATAAATCAGGAGCCGGTGAGCGTGGGTC |
| | | ACGCGGTATCATTGCAGCACTGGGGCCGGATGGTAAGCCCTCCC |
| | | GTATCGTAGTTATCTACACTACGGGGAGTCAGGCAACTATGGAT |
| | | GAACGAAATAGACAGATCGCTGAGATAGGTGCCTCACTGATTAA |
| | | GCATTGGTAAGGATAAATTTCTGGTAAGGAGGACACGTATGGAA |
| | | GTGGGCAAGTTGGGGAAGCCGTATCCGTTGCTGAATCTGGCATA |
| | | TGTGGGAGTATAAGACGCGCAGCGTCGCATCAGGCATTTTTTTC |
| | | TGCGCCAATGCAAAAAGGCCATCCGTCAGGATGGCCTTTCGGCA |
| | | TAACTAGTGAGGCTCCGGTGCCCGTCAGTGGGCAGAGCGCACAT |
| | | CGCCCACAGTCCCCGAGAAGTTGGGGGGAGGGGTCGGCAATTG |
| | | AACCGGTGCCTAGAGAAGGTGGCGCGGGGTAAACTGGGAAAGT |
| | | GATGTCGTGTACTGGCTCCGCCTTTTTCCCGAGGGTGGGGGAGA |
| | | ACCGTATATAAGTGCAGTAGTCGCCGTGAACGTTCTTTTTCGCAA |
| | | CGGGTTTGCCGCCAGAACACAGGTAAGTGCCGTGTGTGGTTCCC |
| | | GCGGGCCTGGCCTCTTTACGGGTTATGGCCCTTGCGTGCCTTGAA |
| | | TTACTTCCACCTGGCTGCAGTACGTGATTCTTGATCCCGAGCTTC |
| | | GGGTTGGAAGTGGGTGGGAGAGTTCGAGGCCTTGCGCTTAAGGA |
| | | GCCCCTTCGCCTCGTGCTTGAGTTGAGGCCTGGCCTGGGCGCTG |
| | | GGGCCGCCGCGTGCGAATCTGGTGGCACCTTCGCGCCTGTCTCG |
| | | CTGCTTTCGATAAGTCTCTAGCCATTTAAAATTTTTGATGACCTG |
| | | CTGCGACGCTTTTTTTCTGGCAAGATAGTCTTGTAAATGCGGGCC |
| | | AAGACGATCTGCACACTGGTATTTCGGTTTTTGGGGCCGCGGGC |
| | | GGCGACGGGGCCCGTGCGTCCCAGCGCACATGTTCGGCGAGGC |
| | | GGGGCCTGCGAGCGCGGCCACCGAGAATCGGACGGGGGTAGTC |
| | | TCAAGCTGGCCGGCCTGCTCTGGTGCCTGGCCTCGCGCCGCCGT |
| | | GTATCGCCCCGCCCTGGGCGGCAAGGCTGGCCCGGTCGGCACCA |
| | | GTTGCGTGAGCGGAAAGATGGCCGCTTCCCGGCCCTGCTGCAGG |
| | | GAGCTCAAAATGGAGGACGCGGCGCTCGGGAGAGCGGGCGGGT |
| | | GAGTCACCCACACAAAGGAAAAGGGCCTTTCCGTCCTCAGCCGT |
| | | CGCTTCATGTGACTCCACGGAGTACCGGGCGCCGTCCAGGCACC |
| | | TCGATTAGTTCTCGAGCTTTTGGAGTACGTCGTCTTTAGGTTGGG |
| | | GGGAGGGGTTTTATGCGATGGAGTTTCCCCACACTGAGTGGGTG |
| | | GAGACTGAAGTTAGGCCAGCTTGGCACTTGATGTAATTCTCCTT |
| | | GGAATTTGCCCTTTTTGAGTTTGGATCTTGGTTCATTCTCAAGCC |
| | | TCAGACAGTGGTTCAAAGTTTTTTTCTTCCATTTCAGGTGTCGTG |
| | | AAA |
| 12 | P-NR-027 | AGCTAGCTTTAATACGACTCACTATAAGGAAATAAGAGAGAAA |
| | plasmid | AGAAGAGTAAGAAGAAATATAAGAGCCACCATGAAATCCTTGA |
| | | GAGTTTTACTAGTGATCCTGTGGCTTCAGTTGAGCTGGGTTTGGA |
| | | GCCAACAGAAGGAGGTGGAGCAGAATTCTGGACCCCTCAGTGTT |
| | | CCAGAGGGAGCCATTGCCTCTCTCAACTGCACTTACAGTGACCG |
| | | AGGTTCCCAGTCCTTCTTCTGGTACAGACAATATTCTGGGAAAA |
| | | GCCCTGAGTTGATAATGTCCATATACTCCAATGGTGACAAAGAA |
| | | GATGGAAGGTTTACAGCACAGCTCAATAAAGCCAGCCAGTATGT |
| | | TTCTCTGCTCATCAGAGACTCCCAGCCCAGTGATTCAGCCACCTA |
| | | CCTCTGTGCCGTGAACGTCCCCTTGAGTTACCAACTCACTTTCGG |
| | | GAAGGGGACCAAACTCTCGGTCATACCAAATGCCGGATCTAGTA |
| | | CAGGTAGCTCCACTGGGCCGGGTAGCACATCAGTTGATGAGTTG |
| | | CAGGCCGAGGTGGACCAGCTTCAAGATGAGAACTACGCTCTGA |
| | | AAACGAAAGTAGCACAGTTGCGAAAAAAGGTAGAGAAGCTCGC |
| | | GTCTGGTGGATGTGGTGGAGAGTTTGATGCTCCAAGCCCTCTCC |
| | | CAGAGACTACAGAGAACGTGGTGTGTGCCCTGGGCCTGACTGTG |
| | | GGTCTGGTGGGCATCATTATTGGGACCATCTTCATCATCAGGAG |
| | | TAAGAGGAGCAGGCTCCTGCACAGTGACTACATGAACATGACTC |
| | | CCCGCCGCCCCGGGCCCACCCGCAAGCATTACCAGCCCTATGCC |
| | | CCACCACGCGACTTCGCAGCCTATCGCTCCAGAGTGAAGTTCAG |
| | | CAGGAGCGCAGACGCCCCCGCGTACCAGCAGGGCCAGAACCAG |
| | | CTCTATAACGAGCTCAATCTAGGACGAAGAGAGGAGTACGATGT |
| | | TTTGGACAAGAGACGTGGCCGGGACCCTGAGATGGGGGGAAAG |
| | | CCGCAGAGAAGGAAGAACCCTCAGGAAGGCCTGTACAATGAAC |
| | | TGCAGAAAGATAAGATGGCGGAGGCCTACAGTGAGATTGGGAT |
| | | GAAAGGCGAGCGCCGGAGGGGCAAGGGGCACGATGGCCTTTAC |
| | | CAGGGTCTCAGTACAGCCACCAAGGACACCTACGACGCCCTTCA |
| | | CATGCAGGCCCTGCCCCCTCGCTGATTAATTAAGCTGCCTTCTGC |
| | | GGGGCTTGCCTTCTGGCCATGCCCTTCTTCTCTCCCTTGCACCTG |
| | | TACCTCTTGGTCTTTGAATAAAGCCTGAGTAGGAAGAAAAAAAA |
| | | AAAAAAAAAAAAAAAGCAGGTGGCGGCCGCAGGTAAGCCAGCC |
| | | CAGGCCTCGCCCTCCAGCTCAAGGCGGGACAGGTGCCCTAGAGT |
| | | AGCCTGCATCCAGGGACAGGCCCCAGCCGGGTGCTGACACGTCC |
| | | ACCTCCATCTCTTCCTCAGGTCTGCCCGGGTGGCATCCCTGTGAC |
| | | CCCTCCCCAGTGCCTCTCCTGGTCGTGGAAGGTGCTACTCCAGTG |
| | | CCCACCAGCCTTGTCCTAATAAAATTAAGTTGCATCATTTTGTTT |
| | | GACTAGGTGTCCTTGTATAATATTATGGGGTGGAGGCGGGTGGT |
| | | ATGGAGCAAGGGGCCCAAGTTAACTTGTTTATTGCAGCTTATAA |
| | | TGGTTACAAATAAAGCAATAGCATCACAAATTTCACAAATAAAG |
| | | CATTTTTTTCACTGCATTCTAGTTGTGGTTTGTCCAAACTCATCA |
| | | ATGTATCTTATCATGTCTGGATCCGCTTCAGGCACCGGGCTTGCG |
| | | GGTCATGCACCAGGTGCGCGGTCCTTCGGGCACCTCGACGTCGG |
| | | CGGTGACGGTGAAGCCGAGCCGCTCGTAGAAGGGGAGGTTGCG |
| | | GGGCGCGGAGGTCTCCAGGAAGGCGGGCACCCCGGCGCGCTCG |
| | | GCCGCCTCCACTCCGGGGAGCACGACGGCGCTGCCCAGACCCTT |
| | | GCCCTGGTGGTCGGGCGAGACGCCGACGGTGGCCAGGAACCAC |
| | | GCGGGCTCCTTGGGCCGGTGCGGCGCCAGGAGGCCTTCCATCTG |
| | | TTGCTGCGCGGCCAGCCTGGAACCGCTCAACTCGGCCATGCGCG |
| | | GGCCGATCTCGGCGAACACCGCCCCCGCTTCGACGCTCTCCGGC |
| | | GTGGTCCAGACCGCCACCGCGGCGCCGTCGTCCGCGACCCACAC |
| | | CTTGCCGATGTCGAGCCCGACGCGCGTGAGGAAGAGTTCTTGCA |
| | | GCTCGGTGACCCGCTCGATGTGGCGGTCCGGGTCGACGGTGTGG |
| | | CGCGTGGCGGGGTAGTCGGCGAACGCGGCGGCGAGGGTGCGTA |
| | | CGGCCCGGGGGACGTCGTCGCGGGTGGCGAGGCGCACCGTGGG |
| | | CTTGTACTCGGTCATGGTGGCCTGCAGAGTCGCTCTGTGTTCGAG |
| | | GCCACACGCGTCACCTTAATATGCGAAGTGGACCTGGGACCGCG |
| | | CCGCCCCGACTGCATCTGCGTGTTTTCGCCAATGACAAGACGCT |
| | | GGGCGGGGTTTGTGTCATCATAGAACTAAAGACATGCAAATATA |
| | | TTTCTTCCGGGGACACCGCCAGCAAACGCGAGCAACGGGCCACG |
| | | GGGATGAAGCAGCTGCGCCACTCCCTGAAGATCCATCGTCTCCT |
| | | AACAAGTTACATCACTCCTGCCCTTCCTCACCCTCATCTCCATCA |
| | | CCTCCTTCATCTCCGTCATCTCCGTCATCACCCTCCGCGGCAGCC |
| | | CCTTCCACCATAGGTGGAAACCAGGGAGGCAAATCTACTCCATC |
| | | GTCAAAGCTGCACACAGTCACCCTGATATTGCAGGTAGGAGCGG |
| | | GCTTTGTCATAACAAGGTCCTTAATCGCATCCTTCAAAACCTCAG |
| | | CAAATATATGAGTTTGTAAAAAGACCATGAAATAACAGACAATG |
| | | GACTCCCTTAGCGGGCCAGGTTGTGGGCCGGGTCCAGGGGCCAT |
| | | TCCAAAGGGGAGACGACTCAATGGTGTAAGACGACATTGTGGA |
| | | ATAGCAAGGGCAGTTCCTCGCCTTAGGTTGTAAAGGGAGGTCTT |
| | | ACTACCTCCATATACGAACACACCGGCGACCCAAGTTCCTTCGT |
| | | CGGTAGTCCTTTCTACGTGACTCCTAGCCAGGAGAGCTCTTAAA |
| | | CCTTCTGCAATGTTCTCAAATTTCGGGTTGGAACCTCCTTGACCA |
| | | CGATGCTTTCCAAACCACCCTCCTTTTTTGCGCCTGCCTCCATCA |
| | | CCCTGACCCCCGCTGCGCGGGGGCACGTCAGGCTCACCATCTGG |
| | | GCCGCCTTCTTGGTGGTATTCAAAATAATCGGCTTCCCCTACAGG |
| | | GTGGAAAAATGGCCTTCTACCTGGAGGGGGCCTGCGCGGTGGA |
| | | GACCCGGATGATGATGACTGACTACTGGGACTCCTGGGCCTCTT |
| | | TTCTCCACGTCCACGACCTCTCCCCCTGGCTCTTTCACGACTTCC |
| | | CCCCCTGGCTCTTTCACGTCCTCTACCCCGGCGGCCTCCACTACC |
| | | TCCTCGACCCCGGCCTCCACTACCTCCTCGACCCCGGCCTCCACT |
| | | GCCTCCTCGACCCCGGCCTCCACCTCCTGCTCCTGCCCCTCCCGC |
| | | TCCTGCTCCTGCTCCTGTTCCACCGTGGGTCCCTTTGCAGCCAAT |
| | | GCAACTTGGACGTTTTTGGGGTCTCCGGACACCATCTCTATGTCT |
| | | TGGCCCTGATCCTGAGCCGCCCGGGGCTCCTGGTCTTCCGCCTCC |
| | | TCGTCCTCGTCCTCTTCCCCGTCCTCGTCCATGTGCCATGATGGC |
| | | GGCCTGCAGCTGTGTTCGAGGCCGCGCGTGTCACCTTAATATGC |
| | | GAAGTGGACCTGGGACCGCGCCGCCCCGACTGCATCTGCGTGTT |
| | | CGAGTTCGCCAATGACAAGACGCTGGGCGGGGAGATCCCCCTTA |
| | | TTAACCCTAAACGGGTAGCATATGCTTCCCGGGTAGTAGTATAT |
| | | ACTATCCAGACTAACCCTAATTCAATAGCATATGTTACCCAACG |
| | | GGAAGCATATGCTATCGAATTAGGGTTAGTAAAAGGGTCCTAAG |
| | | GAACAGCGATCTGGATAGCATATGCTATCCTAATCTATATCTGG |
| | | GTAGCATATGCTATCCTAATCTATATCTGGGTAGCATAGGCTATC |
| | | CTAATCTATATCTGGGTAGCATATGCTATCCTAATCTATATCTGG |
| | | GTAGTATATGCTATCCTAATTTATATCTGGGTAGCATAGGCTATC |
| | | CTAATCTATATCTGGGTAGCATATGCTATCCTAATCTATATCTGG |
| | | GTAGTATATGCTATCCTAATCTGTATCCGGGTAGCATATGCTATC |
| | | CTCATGCATATACAGTCAGCATATGATACCCAGTAGTAGAGTGG |
| | | GAGTGCTATCCTTTGCATATGCCGCCACCTCCCAAGGAGATCTG |
| | | TCGACATCGATGGGCGCGGGTGTACACTCCGCCCATCCCGCCCC |
| | | TAACTCCGCCCAGTTCCGCCCATTCTCCGCCTCATGGCTGACTAA |
| | | TTTTTTTTATTTATGCAGAGGCCGAGGCCGCCTCGGCCTCTGAGC |
| | | TATTCCAGAAGTAGTGAGGAGGCTTTTTTGGAGGCCTAGGCTTTT |
| | | GCAAAAAGCTAATTCGGCGTAATCTGCTGCTTGCAAACAAAAAA |
| | | ACCACCGCTACCAGCGGTGGTTTGTTTGCCGGATCAAGAGCTAC |
| | | CAACTCTTTTTCCGAAGGTAACTGGCTTCAGCAGAGCGCAGATA |
| | | CCAAATACTGTCCTTCTAGTGTAGCCGTAGTTAGGCCACCACTTC |
| | | AAGAACTCTGTAGCACCGCCTACATACCTCGCTCTGCTGAAGCC |
| | | AGTTACCAGTGGCTGCTGCCAGTGGCGATAAGTCGTGTCTTACC |
| | | GGGTTGGACTCAAGAGATAGTTACCGGATAAGGCGCAGCGGTC |
| | | GGGCTGAACGGGGGGTTCGTGCACACAGCCCAGCTTGGAGCGA |
| | | ACGACCTACACCGAACTGAGATACCTACAGCGTGAGCTATGAGA |
| | | AAGCGCCACGCTTCCCGAAGGGAGAAAGGCGGACAGGTATCCG |
| | | GTAAGCGGCAGGGTCGGAACAGGAGAGCGCACGAGGGAGCTTC |
| | | CAGGGGGAAACGCCTGGTATCTTTATAGTCCTGTCGGGTTTCGC |
| | | CACCTCTGACTTGAGCGTCGATTTTTGTGATGCTCGTCAGGGGGG |
| | | CGGAGCCTATGGAAAAACGCCAGCAACGCAAGCTAGAGTTTAA |
| | | ACTTGACAGATGAGACAATAACCCTGATAAATGCTTCAATAATA |
| | | TTGAAAAAGGAAAAGTATGAGTATTCAACATTTCCGTGTCGCCC |
| | | TTATTCCCTTTTTTGCGGCATTTTGCCTTCCTGTTTTTGCTCACCC |
| | | AGAAACGCTGGTGAAAGTAAAAGATGCAGAAGATCACTTGGGT |
| | | GCGCGAGTGGGTTACATCGAACTGGATCTCAACAGCGGTAAGAT |
| | | CCTTGAGAGTTTTCGCCCCGAAGAACGTTTCCCAATGATGAGCA |
| | | CTTTTAAAGTTCTGCTATGTGGCGCGGTATTATCCCGTATTGATG |
| | | CCGGGCAAGAGCAACTCGGTCGCCGCATACACTATTCTCAGAAT |
| | | GACTTGGTTGAATACTCACCAGTCACAGAAAAGCATCTTACGGA |
| | | TGGCATGACAGTAAGAGAATTATGCAGTGCTGCCATAACCATGA |
| | | GTGATAACACTGCGGCCAACTTACTTCTGACAACTATCGGAGGA |
| | | CCGAAGGAGCTAACCGCTTTTTTGCACAACATGGGGGATCATGT |
| | | AACTCGCCTTGATCGTTGGGAACCGGAGCTGAATGAAGCCATAC |
| | | CAAACGACGAGCGTGACACCACGATGCCTGTAGCAATGGCAAC |
| | | AACGTTGCGAAAACTATTAACTGGCGAACTACTTACTCTAGCTT |
| | | CCCGGCAACAACTAATAGACTGGATGGAGGCGGATAAAGTTGC |
| | | AGGACCACTTCTGCGCTCGGCACTTCCGGCTGGCTGGTTTATTGC |
| | | TGATAAATCAGGAGCCGGTGAGCGTGGGTCACGCGGTATCATTG |
| | | CAGCACTGGGGCCGGATGGTAAGCCCTCCCGTATCGTAGTTATC |
| | | TACACTACGGGGAGTCAGGCAACTATGGATGAACGAAATAGAC |
| | | AGATCGCTGAGATAGGTGCCTCACTGATTAAGCATTGGTAAGGA |
| | | TAAATTTCTGGTAAGGAGGACACGTATGGAAGTGGGCAAGTTGG |
| | | GGAAGCCGTATCCGTTGCTGAATCTGGCATATGTGGGAGTATAA |
| | | GACGCGCAGCGTCGCATCAGGCATTTTTTTCTGCGCCAATGCAA |
| | | AAAGGCCATCCGTCAGGATGGCCTTTCGGCATAACTAGTGAGGC |
| | | TCCGGTGCCCGTCAGTGGGCAGAGCGCACATCGCCCACAGTCCC |
| | | CGAGAAGTTGGGGGGAGGGGTCGGCAATTGAACCGGTGCCTAG |
| | | AGAAGGTGGCGCGGGGTAAACTGGGAAAGTGATGTCGTGTACT |
| | | GGCTCCGCCTTTTTCCCGAGGGTGGGGGAGAACCGTATATAAGT |
| | | GCAGTAGTCGCCGTGAACGTTCTTTTTCGCAACGGGTTTGCCGCC |
| | | AGAACACAGGTAAGTGCCGTGTGTGGTTCCCGCGGGCCTGGCCT |
| | | CTTTACGGGTTATGGCCCTTGCGTGCCTTGAATTACTTCCACCTG |
| | | GCTGCAGTACGTGATTCTTGATCCCGAGCTTCGGGTTGGAAGTG |
| | | GGTGGGAGAGTTCGAGGCCTTGCGCTTAAGGAGCCCCTTCGCCT |
| | | CGTGCTTGAGTTGAGGCCTGGCCTGGGCGCTGGGGCCGCCGCGT |
| | | GCGAATCTGGTGGCACCTTCGCGCCTGTCTCGCTGCTTTCGATAA |
| | | GTCTCTAGCCATTTAAAATTTTTGATGACCTGCTGCGACGCTTTT |
| | | TTTCTGGCAAGATAGTCTTGTAAATGCGGGCCAAGACGATCTGC |
| | | ACACTGGTATTTCGGTTTTTGGGGCCGCGGGCGGCGACGGGGCC |
| | | CGTGCGTCCCAGCGCACATGTTCGGCGAGGCGGGGCCTGCGAGC |
| | | GCGGCCACCGAGAATCGGACGGGGGTAGTCTCAAGCTGGCCGG |
| | | CCTGCTCTGGTGCCTGGCCTCGCGCCGCCGTGTATCGCCCCGCCC |
| | | TGGGCGGCAAGGCTGGCCCGGTCGGCACCAGTTGCGTGAGCGG |
| | | AAAGATGGCCGCTTCCCGGCCCTGCTGCAGGGAGCTCAAAATGG |
| | | AGGACGCGGCGCTCGGGAGAGCGGGCGGGTGAGTCACCCACAC |
| | | AAAGGAAAAGGGCCTTTCCGTCCTCAGCCGTCGCTTCATGTGAC |
| | | TCCACGGAGTACCGGGCGCCGTCCAGGCACCTCGATTAGTTCTC |
| | | GAGCTTTTGGAGTACGTCGTCTTTAGGTTGGGGGGAGGGGTTTT |
| | | ATGCGATGGAGTTTCCCCACACTGAGTGGGTGGAGACTGAAGTT |
| | | AGGCCAGCTTGGCACTTGATGTAATTCTCCTTGGAATTTGCCCTT |
| | | TTTGAGTTTGGATCTTGGTTCATTCTCAAGCCTCAGACAGTGGTT |
| | | CAAAGTTTTTTTCTTCCATTTCAGGTGTCGTGAAA |
| 13 | P-NR-024 | AGCTAGCTTTAATACGACTCACTATAAGGAAATAAGAGAGAAA |
| | plasmid | AGAAGAGTAAGAAGAAATATAAGAGCCACCATGGGCTAGTACA |
| | | GATATCAGGATCTACCAGCGGCACGGGCTCTACTACGGTGGCCC |
| | | AACTCAGGGAACGGGTCAAGACACTGAGAGCCCAAAATTACGA |
| | | ATTGGAGTCTGAGGTTCAACGCCTTCGAGAGCAAGTTGCGCAGC |
| | | TTGCAAGTGGTGGATGTGGTGGAAGAGCACGGTCTGAATCTGCA |
| | | CAGAGCAAGATGCTGAGTGGAGTCGGGGGCTTTGTGCTGGGCCT |
| | | GCTCTTCCTTGGGGCCGGGCTGTTCATCAGGAGTAAGAGGAGCA |
| | | GGCTCCTGCACAGTGACTACATGAACATGACTCCCCGCCGCCCC |
| | | GGGCCCACCCGCAAGCATTACCAGCCCTATGCCCCACCACGCGA |
| | | CTTCGCAGCCTATCGCTCCAGAGTGAAGTTCAGCAGGAGCGCAG |
| | | ACGCCCCCGCGTACCAGCAGGGCCAGAACCAGCTCTATAACGA |
| | | GCTCAATCTAGGACGAAGAGAGGAGTACGATGTTTTGGACAAG |
| | | AGACGTGGCCGGGACCCTGAGATGGGGGGAAAGCCGCAGAGAA |
| | | GGAAGAACCCTCAGGAAGGCCTGTACAATGAACTGCAGAAAGA |
| | | TAAGATGGCGGAGGCCTACAGTGAGATTGGGATGAAAGGCGAG |
| | | CGCCGGAGGGGCAAGGGGCACGATGGCCTTTACCAGGGTCTCA |
| | | GTACAGCCACCAAGGACACCTACGACGCCCTTCACATGCAGGCC |
| | | CTGCCCCCTCGCTGATTAATTAAGCTGCCTTCTGCGGGGCTTGCC |
| | | TTCTGGCCATGCCCTTCTTCTCTCCCTTGCACCTGTACCTCTTGGT |
| | | CTTTGAATAAAGCCTGAGTAGGAAGAAAAAAAAAAAAAAAAAA |
| | | AAAAAGCAGGTGGCGGCCGCAGGTAAGCCAGCCCAGGCCTCGC |
| | | CCTCCAGCTCAAGGCGGGACAGGTGCCCTAGAGTAGCCTGCATC |
| | | CAGGGACAGGCCCCAGCCGGGTGCTGACACGTCCACCTCCATCT |
| | | CTTCCTCAGGTCTGCCCGGGTGGCATCCCTGTGACCCCTCCCCAG |
| | | TGCCTCTCCTGGTCGTGGAAGGTGCTACTCCAGTGCCCACCAGC |
| | | CTTGTCCTAATAAAATTAAGTTGCATCATTTTGTTTGACTAGGTG |
| | | TCCTTGTATAATATTATGGGGTGGAGGCGGGTGGTATGGAGCAA |
| | | GGGGCCCAAGTTAACTTGTTTATTGCAGCTTATAATGGTTACAA |
| | | ATAAAGCAATAGCATCACAAATTTCACAAATAAAGCATTTTTTT |
| | | CACTGCATTCTAGTTGTGGTTTGTCCAAACTCATCAATGTATCTT |
| | | ATCATGTCTGGATCCGCTTCAGGCACCGGGCTTGCGGGTCATGC |
| | | ACCAGGTGCGCGGTCCTTCGGGCACCTCGACGTCGGCGGTGACG |
| | | GTGAAGCCGAGCCGCTCGTAGAAGGGGAGGTTGCGGGGCGCGG |
| | | AGGTCTCCAGGAAGGCGGGCACCCCGGCGCGCTCGGCCGCCTCC |
| | | ACTCCGGGGAGCACGACGGCGCTGCCCAGACCCTTGCCCTGGTG |
| | | GTCGGGCGAGACGCCGACGGTGGCCAGGAACCACGCGGGCTCC |
| | | TTGGGCCGGTGCGGCGCCAGGAGGCCTTCCATCTGTTGCTGCGC |
| | | GGCCAGCCTGGAACCGCTCAACTCGGCCATGCGCGGGCCGATCT |
| | | CGGCGAACACCGCCCCCGCTTCGACGCTCTCCGGCGTGGTCCAG |
| | | ACCGCCACCGCGGCGCCGTCGTCCGCGACCCACACCTTGCCGAT |
| | | GTCGAGCCCGACGCGCGTGAGGAAGAGTTCTTGCAGCTCGGTGA |
| | | CCCGCTCGATGTGGCGGTCCGGGTCGACGGTGTGGCGCGTGGCG |
| | | GGGTAGTCGGCGAACGCGGCGGCGAGGGTGCGTACGGCCCGGG |
| | | GGACGTCGTCGCGGGTGGCGAGGCGCACCGTGGGCTTGTACTCG |
| | | GTCATGGTGGCCTGCAGAGTCGCTCTGTGTTCGAGGCCACACGC |
| | | GTCACCTTAATATGCGAAGTGGACCTGGGACCGCGCCGCCCCGA |
| | | CTGCATCTGCGTGTTTTCGCCAATGACAAGACGCTGGGCGGGGT |
| | | TTGTGTCATCATAGAACTAAAGACATGCAAATATATTTCTTCCGG |
| | | GGACACCGCCAGCAAACGCGAGCAACGGGCCACGGGGATGAAG |
| | | CAGCTGCGCCACTCCCTGAAGATCCATCGTCTCCTAACAAGTTA |
| | | CATCACTCCTGCCCTTCCTCACCCTCATCTCCATCACCTCCTTCAT |
| | | CTCCGTCATCTCCGTCATCACCCTCCGCGGCAGCCCCTTCCACCA |
| | | TAGGTGGAAACCAGGGAGGCAAATCTACTCCATCGTCAAAGCTG |
| | | CACACAGTCACCCTGATATTGCAGGTAGGAGCGGGCTTTGTCAT |
| | | AACAAGGTCCTTAATCGCATCCTTCAAAACCTCAGCAAATATAT |
| | | GAGTTTGTAAAAAGACCATGAAATAACAGACAATGGACTCCCTT |
| | | AGCGGGCCAGGTTGTGGGCCGGGTCCAGGGGCCATTCCAAAGG |
| | | GGAGACGACTCAATGGTGTAAGACGACATTGTGGAATAGCAAG |
| | | GGCAGTTCCTCGCCTTAGGTTGTAAAGGGAGGTCTTACTACCTC |
| | | CATATACGAACACACCGGCGACCCAAGTTCCTTCGTCGGTAGTC |
| | | CTTTCTACGTGACTCCTAGCCAGGAGAGCTCTTAAACCTTCTGCA |
| | | ATGTTCTCAAATTTCGGGTTGGAACCTCCTTGACCACGATGCTTT |
| | | CCAAACCACCCTCCTTTTTTGCGCCTGCCTCCATCACCCTGACCC |
| | | CCGCTGCGCGGGGGCACGTCAGGCTCACCATCTGGGCCGCCTTC |
| | | TTGGTGGTATTCAAAATAATCGGCTTCCCCTACAGGGTGGAAAA |
| | | ATGGCCTTCTACCTGGAGGGGGCCTGCGCGGTGGAGACCCGGAT |
| | | GATGATGACTGACTACTGGGACTCCTGGGCCTCTTTTCTCCACGT |
| | | CCACGACCTCTCCCCCTGGCTCTTTCACGACTTCCCCCCCTGGCT |
| | | CTTTCACGTCCTCTACCCCGGCGGCCTCCACTACCTCCTCGACCC |
| | | CGGCCTCCACTACCTCCTCGACCCCGGCCTCCACTGCCTCCTCGA |
| | | CCCCGGCCTCCACCTCCTGCTCCTGCCCCTCCCGCTCCTGCTCCT |
| | | GCTCCTGTTCCACCGTGGGTCCCTTTGCAGCCAATGCAACTTGGA |
| | | CGTTTTTGGGGTCTCCGGACACCATCTCTATGTCTTGGCCCTGAT |
| | | CCTGAGCCGCCCGGGGCTCCTGGTCTTCCGCCTCCTCGTCCTCGT |
| | | CCTCTTCCCCGTCCTCGTCCATGTGCCATGATGGCGGCCTGCAGC |
| | | TGTGTTCGAGGCCGCGCGTGTCACCTTAATATGCGAAGTGGACC |
| | | TGGGACCGCGCCGCCCCGACTGCATCTGCGTGTTCGAGTTCGCC |
| | | AATGACAAGACGCTGGGCGGGGAGATCCCCCTTATTAACCCTAA |
| | | ACGGGTAGCATATGCTTCCCGGGTAGTAGTATATACTATCCAGA |
| | | CTAACCCTAATTCAATAGCATATGTTACCCAACGGGAAGCATAT |
| | | GCTATCGAATTAGGGTTAGTAAAAGGGTCCTAAGGAACAGCGAT |
| | | CTGGATAGCATATGCTATCCTAATCTATATCTGGGTAGCATATGC |
| | | TATCCTAATCTATATCTGGGTAGCATAGGCTATCCTAATCTATAT |
| | | CTGGGTAGCATATGCTATCCTAATCTATATCTGGGTAGTATATGC |
| | | TATCCTAATTTATATCTGGGTAGCATAGGCTATCCTAATCTATAT |
| | | CTGGGTAGCATATGCTATCCTAATCTATATCTGGGTAGTATATGC |
| | | TATCCTAATCTGTATCCGGGTAGCATATGCTATCCTCATGCATAT |
| | | ACAGTCAGCATATGATACCCAGTAGTAGAGTGGGAGTGCTATCC |
| | | TTTGCATATGCCGCCACCTCCCAAGGAGATCTGTCGACATCGAT |
| | | GGGCGCGGGTGTACACTCCGCCCATCCCGCCCCTAACTCCGCCC |
| | | AGTTCCGCCCATTCTCCGCCTCATGGCTGACTAATTTTTTTTATTT |
| | | ATGCAGAGGCCGAGGCCGCCTCGGCCTCTGAGCTATTCCAGAAG |
| | | TAGTGAGGAGGCTTTTTTGGAGGCCTAGGCTTTTGCAAAAAGCT |
| | | AATTCGGCGTAATCTGCTGCTTGCAAACAAAAAAACCACCGCTA |
| | | CCAGCGGTGGTTTGTTTGCCGGATCAAGAGCTACCAACTCTTTTT |
| | | CCGAAGGTAACTGGCTTCAGCAGAGCGCAGATACCAAATACTGT |
| | | CCTTCTAGTGTAGCCGTAGTTAGGCCACCACTTCAAGAACTCTGT |
| | | AGCACCGCCTACATACCTCGCTCTGCTGAAGCCAGTTACCAGTG |
| | | GCTGCTGCCAGTGGCGATAAGTCGTGTCTTACCGGGTTGGACTC |
| | | AAGAGATAGTTACCGGATAAGGCGCAGCGGTCGGGCTGAACGG |
| | | GGGGTTCGTGCACACAGCCCAGCTTGGAGCGAACGACCTACACC |
| | | GAACTGAGATACCTACAGCGTGAGCTATGAGAAAGCGCCACGC |
| | | TTCCCGAAGGGAGAAAGGCGGACAGGTATCCGGTAAGCGGCAG |
| | | GGTCGGAACAGGAGAGCGCACGAGGGAGCTTCCAGGGGGAAAC |
| | | GCCTGGTATCTTTATAGTCCTGTCGGGTTTCGCCACCTCTGACTT |
| | | GAGCGTCGATTTTTGTGATGCTCGTCAGGGGGGCGGAGCCTATG |
| | | GAAAAACGCCAGCAACGCAAGCTAGAGTTTAAACTTGACAGAT |
| | | GAGACAATAACCCTGATAAATGCTTCAATAATATTGAAAAAGGA |
| | | AAAGTATGAGTATTCAACATTTCCGTGTCGCCCTTATTCCCTTTT |
| | | TTGCGGCATTTTGCCTTCCTGTTTTTGCTCACCCAGAAACGCTGG |
| | | TGAAAGTAAAAGATGCAGAAGATCACTTGGGTGCGCGAGTGGG |
| | | TTACATCGAACTGGATCTCAACAGCGGTAAGATCCTTGAGAGTT |
| | | TTCGCCCCGAAGAACGTTTCCCAATGATGAGCACTTTTAAAGTT |
| | | CTGCTATGTGGCGCGGTATTATCCCGTATTGATGCCGGGCAAGA |
| | | GCAACTCGGTCGCCGCATACACTATTCTCAGAATGACTTGGTTG |
| | | AATACTCACCAGTCACAGAAAAGCATCTTACGGATGGCATGACA |
| | | GTAAGAGAATTATGCAGTGCTGCCATAACCATGAGTGATAACAC |
| | | TGCGGCCAACTTACTTCTGACAACTATCGGAGGACCGAAGGAGC |
| | | TAACCGCTTTTTTGCACAACATGGGGGATCATGTAACTCGCCTTG |
| | | ATCGTTGGGAACCGGAGCTGAATGAAGCCATACCAAACGACGA |
| | | GCGTGACACCACGATGCCTGTAGCAATGGCAACAACGTTGCGAA |
| | | AACTATTAACTGGCGAACTACTTACTCTAGCTTCCCGGCAACAA |
| | | CTAATAGACTGGATGGAGGCGGATAAAGTTGCAGGACCACTTCT |
| | | GCGCTCGGCACTTCCGGCTGGCTGGTTTATTGCTGATAAATCAG |
| | | GAGCCGGTGAGCGTGGGTCACGCGGTATCATTGCAGCACTGGGG |
| | | CCGGATGGTAAGCCCTCCCGTATCGTAGTTATCTACACTACGGG |
| | | GAGTCAGGCAACTATGGATGAACGAAATAGACAGATCGCTGAG |
| | | ATAGGTGCCTCACTGATTAAGCATTGGTAAGGATAAATTTCTGG |
| | | TAAGGAGGACACGTATGGAAGTGGGCAAGTTGGGGAAGCCGTA |
| | | TCCGTTGCTGAATCTGGCATATGTGGGAGTATAAGACGCGCAGC |
| | | GTCGCATCAGGCATTTTTTTCTGCGCCAATGCAAAAAGGCCATC |
| | | CGTCAGGATGGCCTTTCGGCATAACTAGTGAGGCTCCGGTGCCC |
| | | GTCAGTGGGCAGAGCGCACATCGCCCACAGTCCCCGAGAAGTTG |
| | | GGGGGAGGGGTCGGCAATTGAACCGGTGCCTAGAGAAGGTGGC |
| | | GCGGGGTAAACTGGGAAAGTGATGTCGTGTACTGGCTCCGCCTT |
| | | TTTCCCGAGGGTGGGGGAGAACCGTATATAAGTGCAGTAGTCGC |
| | | CGTGAACGTTCTTTTTCGCAACGGGTTTGCCGCCAGAACACAGG |
| | | TAAGTGCCGTGTGTGGTTCCCGCGGGCCTGGCCTCTTTACGGGTT |
| | | ATGGCCCTTGCGTGCCTTGAATTACTTCCACCTGGCTGCAGTACG |
| | | TGATTCTTGATCCCGAGCTTCGGGTTGGAAGTGGGTGGGAGAGT |
| | | TCGAGGCCTTGCGCTTAAGGAGCCCCTTCGCCTCGTGCTTGAGTT |
| | | GAGGCCTGGCCTGGGCGCTGGGGCCGCCGCGTGCGAATCTGGTG |
| | | GCACCTTCGCGCCTGTCTCGCTGCTTTCGATAAGTCTCTAGCCAT |
| | | TTAAAATTTTTGATGACCTGCTGCGACGCTTTTTTTCTGGCAAGA |
| | | TAGTCTTGTAAATGCGGGCCAAGACGATCTGCACACTGGTATTT |
| | | CGGTTTTTGGGGCCGCGGGCGGCGACGGGGCCCGTGCGTCCCAG |
| | | CGCACATGTTCGGCGAGGCGGGGCCTGCGAGCGCGGCCACCGA |
| | | GAATCGGACGGGGGTAGTCTCAAGCTGGCCGGCCTGCTCTGGTG |
| | | CCTGGCCTCGCGCCGCCGTGTATCGCCCCGCCCTGGGCGGCAAG |
| | | GCTGGCCCGGTCGGCACCAGTTGCGTGAGCGGAAAGATGGCCG |
| | | CTTCCCGGCCCTGCTGCAGGGAGCTCAAAATGGAGGACGCGGCG |
| | | CTCGGGAGAGCGGGCGGGTGAGTCACCCACACAAAGGAAAAGG |
| | | GCCTTTCCGTCCTCAGCCGTCGCTTCATGTGACTCCACGGAGTAC |
| | | CGGGCGCCGTCCAGGCACCTCGATTAGTTCTCGAGCTTTTGGAG |
| | | TACGTCGTCTTTAGGTTGGGGGGAGGGGTTTTATGCGATGGAGT |
| | | TTCCCCACACTGAGTGGGTGGAGACTGAAGTTAGGCCAGCTTGG |
| | | CACTTGATGTAATTCTCCTTGGAATTTGCCCTTTTTGAGTTTGGA |
| | | TCTTGGTTCATTCTCAAGCCTCAGACAGTGGTTCAAAGTTTTTTT |
| | | CTTCCATTTCAGGTGTCGTGAAA |
| 14 | P-NR-028 | AGCTAGCTTTAATACGACTCACTATAAGGAAATAAGAGAGAAA |
| | plasmid | AGAAGAGTAAGAAGAAATATAAGAGCCACCATGCTGAGTCTTCT |
| | | GCTCCTTCTCCTGGGACTAGGCTCTGTGTTCAGTGCTGTCATCTC |
| | | TCAAAAGCCAAGCAGGGATATCTGTCAACGTGGAACCTCCCTGA |
| | | CGATCCAGTGTCAAGTCGATAGCCAAGTCACCATGATGTTCTGG |
| | | TACCGTCAGCAACCTGGACAGAGCCTGACACTGATCGCAACTGC |
| | | AAATCAGGGCTCTGAGGCCACATATGAGAGTGGATTTGTCATTG |
| | | ACAAGTTTCCCATCAGCCGCCCAAACCTAACATTCTCAACTCTG |
| | | ACTGTGAGCAACATGAGCCCTGAAGACAGCAGCATATATCTCTG |
| | | CAGCGTTGCCGGGACTATCGACGAGCAGTACTTCGGGCCGGGCA |
| | | CCAGGCTCACGGTCACAGAGAGTGGGGGCACATCAGGATCTAC |
| | | CAGCGGCACGGGCTCTACTACGGTGGCCCAACTCAGGGAACGG |
| | | GTCAAGACACTGAGAGCCCAAAATTACGAATTGGAGTCTGAGGT |
| | | TCAACGCCTTCGAGAGCAAGTTGCGCAGCTTGCAAGTGGTGGAT |
| | | GTGGTGGAAGAGCACGGTCTGAATCTGCACAGAGCAAGATGCT |
| | | GAGTGGAGTCGGGGGCTTTGTGCTGGGCCTGCTCTTCCTTGGGG |
| | | CCGGGCTGTTCATCAGGAGTAAGAGGAGCAGGCTCCTGCACAGT |
| | | GACTACATGAACATGACTCCCCGCCGCCCCGGGCCCACCCGCAA |
| | | GCATTACCAGCCCTATGCCCCACCACGCGACTTCGCAGCCTATC |
| | | GCTCCAGAGTGAAGTTCAGCAGGAGCGCAGACGCCCCCGCGTA |
| | | CCAGCAGGGCCAGAACCAGCTCTATAACGAGCTCAATCTAGGAC |
| | | GAAGAGAGGAGTACGATGTTTTGGACAAGAGACGTGGCCGGGA |
| | | CCCTGAGATGGGGGGAAAGCCGCAGAGAAGGAAGAACCCTCAG |
| | | GAAGGCCTGTACAATGAACTGCAGAAAGATAAGATGGCGGAGG |
| | | CCTACAGTGAGATTGGGATGAAAGGCGAGCGCCGGAGGGGCAA |
| | | GGGGCACGATGGCCTTTACCAGGGTCTCAGTACAGCCACCAAGG |
| | | ACACCTACGACGCCCTTCACATGCAGGCCCTGCCCCCTCGCTGA |
| | | TTAATTAAGCTGCCTTCTGCGGGGCTTGCCTTCTGGCCATGCCCT |
| | | TCTTCTCTCCCTTGCACCTGTACCTCTTGGTCTTTGAATAAAGCC |
| | | TGAGTAGGAAGAAAAAAAAAAAAAAAAAAAAAAAGCAGGTGG |
| | | CGGCCGCAGGTAAGCCAGCCCAGGCCTCGCCCTCCAGCTCAAGG |
| | | CGGGACAGGTGCCCTAGAGTAGCCTGCATCCAGGGACAGGCCC |
| | | CAGCCGGGTGCTGACACGTCCACCTCCATCTCTTCCTCAGGTCTG |
| | | CCCGGGTGGCATCCCTGTGACCCCTCCCCAGTGCCTCTCCTGGTC |
| | | GTGGAAGGTGCTACTCCAGTGCCCACCAGCCTTGTCCTAATAAA |
| | | ATTAAGTTGCATCATTTTGTTTGACTAGGTGTCCTTGTATAATAT |
| | | TATGGGGTGGAGGCGGGTGGTATGGAGCAAGGGGCCCAAGTTA |
| | | ACTTGTTTATTGCAGCTTATAATGGTTACAAATAAAGCAATAGC |
| | | ATCACAAATTTCACAAATAAAGCATTTTTTTCACTGCATTCTAGT |
| | | TGTGGTTTGTCCAAACTCATCAATGTATCTTATCATGTCTGGATC |
| | | CGCTTCAGGCACCGGGCTTGCGGGTCATGCACCAGGTGCGCGGT |
| | | CCTTCGGGCACCTCGACGTCGGCGGTGACGGTGAAGCCGAGCCG |
| | | CTCGTAGAAGGGGAGGTTGCGGGGCGCGGAGGTCTCCAGGAAG |
| | | GCGGGCACCCCGGCGCGCTCGGCCGCCTCCACTCCGGGGAGCAC |
| | | GACGGCGCTGCCCAGACCCTTGCCCTGGTGGTCGGGCGAGACGC |
| | | CGACGGTGGCCAGGAACCACGCGGGCTCCTTGGGCCGGTGCGG |
| | | CGCCAGGAGGCCTTCCATCTGTTGCTGCGCGGCCAGCCTGGAAC |
| | | CGCTCAACTCGGCCATGCGCGGGCCGATCTCGGCGAACACCGCC |
| | | CCCGCTTCGACGCTCTCCGGCGTGGTCCAGACCGCCACCGCGGC |
| | | GCCGTCGTCCGCGACCCACACCTTGCCGATGTCGAGCCCGACGC |
| | | GCGTGAGGAAGAGTTCTTGCAGCTCGGTGACCCGCTCGATGTGG |
| | | CGGTCCGGGTCGACGGTGTGGCGCGTGGCGGGGTAGTCGGCGA |
| | | ACGCGGCGGCGAGGGTGCGTACGGCCCGGGGGACGTCGTCGCG |
| | | GGTGGCGAGGCGCACCGTGGGCTTGTACTCGGTCATGGTGGCCT |
| | | GCAGAGTCGCTCTGTGTTCGAGGCCACACGCGTCACCTTAATAT |
| | | GCGAAGTGGACCTGGGACCGCGCCGCCCCGACTGCATCTGCGTG |
| | | TTTTCGCCAATGACAAGACGCTGGGCGGGGTTTGTGTCATCATA |
| | | GAACTAAAGACATGCAAATATATTTCTTCCGGGGACACCGCCAG |
| | | CAAACGCGAGCAACGGGCCACGGGGATGAAGCAGCTGCGCCAC |
| | | TCCCTGAAGATCCATCGTCTCCTAACAAGTTACATCACTCCTGCC |
| | | CTTCCTCACCCTCATCTCCATCACCTCCTTCATCTCCGTCATCTCC |
| | | GTCATCACCCTCCGCGGCAGCCCCTTCCACCATAGGTGGAAACC |
| | | AGGGAGGCAAATCTACTCCATCGTCAAAGCTGCACACAGTCACC |
| | | CTGATATTGCAGGTAGGAGCGGGCTTTGTCATAACAAGGTCCTT |
| | | AATCGCATCCTTCAAAACCTCAGCAAATATATGAGTTTGTAAAA |
| | | AGACCATGAAATAACAGACAATGGACTCCCTTAGCGGGCCAGG |
| | | TTGTGGGCCGGGTCCAGGGGCCATTCCAAAGGGGAGACGACTC |
| | | AATGGTGTAAGACGACATTGTGGAATAGCAAGGGCAGTTCCTCG |
| | | CCTTAGGTTGTAAAGGGAGGTCTTACTACCTCCATATACGAACA |
| | | CACCGGCGACCCAAGTTCCTTCGTCGGTAGTCCTTTCTACGTGAC |
| | | TCCTAGCCAGGAGAGCTCTTAAACCTTCTGCAATGTTCTCAAATT |
| | | TCGGGTTGGAACCTCCTTGACCACGATGCTTTCCAAACCACCCTC |
| | | CTTTTTTGCGCCTGCCTCCATCACCCTGACCCCCGCTGCGCGGGG |
| | | GCACGTCAGGCTCACCATCTGGGCCGCCTTCTTGGTGGTATTCA |
| | | AAATAATCGGCTTCCCCTACAGGGTGGAAAAATGGCCTTCTACC |
| | | TGGAGGGGGCCTGCGCGGTGGAGACCCGGATGATGATGACTGA |
| | | CTACTGGGACTCCTGGGCCTCTTTTCTCCACGTCCACGACCTCTC |
| | | CCCCTGGCTCTTTCACGACTTCCCCCCCTGGCTCTTTCACGTCCT |
| | | CTACCCCGGCGGCCTCCACTACCTCCTCGACCCCGGCCTCCACTA |
| | | CCTCCTCGACCCCGGCCTCCACTGCCTCCTCGACCCCGGCCTCCA |
| | | CCTCCTGCTCCTGCCCCTCCCGCTCCTGCTCCTGCTCCTGTTCCAC |
| | | CGTGGGTCCCTTTGCAGCCAATGCAACTTGGACGTTTTTGGGGTC |
| | | TCCGGACACCATCTCTATGTCTTGGCCCTGATCCTGAGCCGCCCG |
| | | GGGCTCCTGGTCTTCCGCCTCCTCGTCCTCGTCCTCTTCCCCGTC |
| | | CTCGTCCATGTGCCATGATGGCGGCCTGCAGCTGTGTTCGAGGC |
| | | CGCGCGTGTCACCTTAATATGCGAAGTGGACCTGGGACCGCGCC |
| | | GCCCCGACTGCATCTGCGTGTTCGAGTTCGCCAATGACAAGACG |
| | | CTGGGCGGGGAGATCCCCCTTATTAACCCTAAACGGGTAGCATA |
| | | TGCTTCCCGGGTAGTAGTATATACTATCCAGACTAACCCTAATTC |
| | | AATAGCATATGTTACCCAACGGGAAGCATATGCTATCGAATTAG |
| | | GGTTAGTAAAAGGGTCCTAAGGAACAGCGATCTGGATAGCATAT |
| | | GCTATCCTAATCTATATCTGGGTAGCATATGCTATCCTAATCTAT |
| | | ATCTGGGTAGCATAGGCTATCCTAATCTATATCTGGGTAGCATAT |
| | | GCTATCCTAATCTATATCTGGGTAGTATATGCTATCCTAATTTAT |
| | | ATCTGGGTAGCATAGGCTATCCTAATCTATATCTGGGTAGCATAT |
| | | GCTATCCTAATCTATATCTGGGTAGTATATGCTATCCTAATCTGT |
| | | ATCCGGGTAGCATATGCTATCCTCATGCATATACAGTCAGCATA |
| | | TGATACCCAGTAGTAGAGTGGGAGTGCTATCCTTTGCATATGCC |
| | | GCCACCTCCCAAGGAGATCTGTCGACATCGATGGGCGCGGGTGT |
| | | ACACTCCGCCCATCCCGCCCCTAACTCCGCCCAGTTCCGCCCATT |
| | | CTCCGCCTCATGGCTGACTAATTTTTTTTATTTATGCAGAGGCCG |
| | | AGGCCGCCTCGGCCTCTGAGCTATTCCAGAAGTAGTGAGGAGGC |
| | | TTTTTTGGAGGCCTAGGCTTTTGCAAAAAGCTAATTCGGCGTAAT |
| | | CTGCTGCTTGCAAACAAAAAAACCACCGCTACCAGCGGTGGTTT |
| | | GTTTGCCGGATCAAGAGCTACCAACTCTTTTTCCGAAGGTAACT |
| | | GGCTTCAGCAGAGCGCAGATACCAAATACTGTCCTTCTAGTGTA |
| | | GCCGTAGTTAGGCCACCACTTCAAGAACTCTGTAGCACCGCCTA |
| | | CATACCTCGCTCTGCTGAAGCCAGTTACCAGTGGCTGCTGCCAG |
| | | TGGCGATAAGTCGTGTCTTACCGGGTTGGACTCAAGAGATAGTT |
| | | ACCGGATAAGGCGCAGCGGTCGGGCTGAACGGGGGGTTCGTGC |
| | | ACACAGCCCAGCTTGGAGCGAACGACCTACACCGAACTGAGAT |
| | | ACCTACAGCGTGAGCTATGAGAAAGCGCCACGCTTCCCGAAGG |
| | | GAGAAAGGCGGACAGGTATCCGGTAAGCGGCAGGGTCGGAACA |
| | | GGAGAGCGCACGAGGGAGCTTCCAGGGGGAAACGCCTGGTATC |
| | | TTTATAGTCCTGTCGGGTTTCGCCACCTCTGACTTGAGCGTCGAT |
| | | TTTTGTGATGCTCGTCAGGGGGGCGGAGCCTATGGAAAAACGCC |
| | | AGCAACGCAAGCTAGAGTTTAAACTTGACAGATGAGACAATAA |
| | | CCCTGATAAATGCTTCAATAATATTGAAAAAGGAAAAGTATGAG |
| | | TATTCAACATTTCCGTGTCGCCCTTATTCCCTTTTTTGCGGCATTT |
| | | TGCCTTCCTGTTTTTGCTCACCCAGAAACGCTGGTGAAAGTAAA |
| | | AGATGCAGAAGATCACTTGGGTGCGCGAGTGGGTTACATCGAAC |
| | | TGGATCTCAACAGCGGTAAGATCCTTGAGAGTTTTCGCCCCGAA |
| | | GAACGTTTCCCAATGATGAGCACTTTTAAAGTTCTGCTATGTGGC |
| | | GCGGTATTATCCCGTATTGATGCCGGGCAAGAGCAACTCGGTCG |
| | | CCGCATACACTATTCTCAGAATGACTTGGTTGAATACTCACCAG |
| | | TCACAGAAAAGCATCTTACGGATGGCATGACAGTAAGAGAATTA |
| | | TGCAGTGCTGCCATAACCATGAGTGATAACACTGCGGCCAACTT |
| | | ACTTCTGACAACTATCGGAGGACCGAAGGAGCTAACCGCTTTTT |
| | | TGCACAACATGGGGGATCATGTAACTCGCCTTGATCGTTGGGAA |
| | | CCGGAGCTGAATGAAGCCATACCAAACGACGAGCGTGACACCA |
| | | CGATGCCTGTAGCAATGGCAACAACGTTGCGAAAACTATTAACT |
| | | GGCGAACTACTTACTCTAGCTTCCCGGCAACAACTAATAGACTG |
| | | GATGGAGGCGGATAAAGTTGCAGGACCACTTCTGCGCTCGGCAC |
| | | TTCCGGCTGGCTGGTTTATTGCTGATAAATCAGGAGCCGGTGAG |
| | | CGTGGGTCACGCGGTATCATTGCAGCACTGGGGCCGGATGGTAA |
| | | GCCCTCCCGTATCGTAGTTATCTACACTACGGGGAGTCAGGCAA |
| | | CTATGGATGAACGAAATAGACAGATCGCTGAGATAGGTGCCTCA |
| | | CTGATTAAGCATTGGTAAGGATAAATTTCTGGTAAGGAGGACAC |
| | | GTATGGAAGTGGGCAAGTTGGGGAAGCCGTATCCGTTGCTGAAT |
| | | CTGGCATATGTGGGAGTATAAGACGCGCAGCGTCGCATCAGGCA |
| | | TTTTTTTCTGCGCCAATGCAAAAAGGCCATCCGTCAGGATGGCC |
| | | TTTCGGCATAACTAGTGAGGCTCCGGTGCCCGTCAGTGGGCAGA |
| | | GCGCACATCGCCCACAGTCCCCGAGAAGTTGGGGGGAGGGGTC |
| | | GGCAATTGAACCGGTGCCTAGAGAAGGTGGCGCGGGGTAAACT |
| | | GGGAAAGTGATGTCGTGTACTGGCTCCGCCTTTTTCCCGAGGGT |
| | | GGGGGAGAACCGTATATAAGTGCAGTAGTCGCCGTGAACGTTCT |
| | | TTTTCGCAACGGGTTTGCCGCCAGAACACAGGTAAGTGCCGTGT |
| | | GTGGTTCCCGCGGGCCTGGCCTCTTTACGGGTTATGGCCCTTGCG |
| | | TGCCTTGAATTACTTCCACCTGGCTGCAGTACGTGATTCTTGATC |
| | | CCGAGCTTCGGGTTGGAAGTGGGTGGGAGAGTTCGAGGCCTTGC |
| | | GCTTAAGGAGCCCCTTCGCCTCGTGCTTGAGTTGAGGCCTGGCC |
| | | TGGGCGCTGGGGCCGCCGCGTGCGAATCTGGTGGCACCTTCGCG |
| | | CCTGTCTCGCTGCTTTCGATAAGTCTCTAGCCATTTAAAATTTTT |
| | | GATGACCTGCTGCGACGCTTTTTTTCTGGCAAGATAGTCTTGTAA |
| | | ATGCGGGCCAAGACGATCTGCACACTGGTATTTCGGTTTTTGGG |
| | | GCCGCGGGCGGCGACGGGGCCCGTGCGTCCCAGCGCACATGTTC |
| | | GGCGAGGCGGGGCCTGCGAGCGCGGCCACCGAGAATCGGACGG |
| | | GGGTAGTCTCAAGCTGGCCGGCCTGCTCTGGTGCCTGGCCTCGC |
| | | GCCGCCGTGTATCGCCCCGCCCTGGGCGGCAAGGCTGGCCCGGT |
| | | CGGCACCAGTTGCGTGAGCGGAAAGATGGCCGCTTCCCGGCCCT |
| | | GCTGCAGGGAGCTCAAAATGGAGGACGCGGCGCTCGGGAGAGC |
| | | GGGCGGGTGAGTCACCCACACAAAGGAAAAGGGCCTTTCCGTC |
| | | CTCAGCCGTCGCTTCATGTGACTCCACGGAGTACCGGGCGCCGT |
| | | CCAGGCACCTCGATTAGTTCTCGAGCTTTTGGAGTACGTCGTCTT |
| | | TAGGTTGGGGGGAGGGGTTTTATGCGATGGAGTTTCCCCACACT |
| | | GAGTGGGTGGAGACTGAAGTTAGGCCAGCTTGGCACTTGATGTA |
| | | ATTCTCCTTGGAATTTGCCCTTTTTGAGTTTGGATCTTGGTTCATT |
| | | CTCAAGCCTCAGACAGTGGTTCAAAGTTTTTTTCTTCCATTTCAG |
| | | GTGTCGTGAAA |
| 15 | Vα-1 | MKSLRVLLVILWLQLSWVWSQQKEVEQNSGPLSVPEGAIASLNCT |
| | | YSDRGSQSFFWYRQYSGKSPELIMSIYSNGDKEDGRFTAQLNKASQ |
| | | YVSLLIRDSQPSDSATYLCAVNVPLSYQLTFGKGTKLSVIP |
| 16 | Vβ-1 | MLSLLLLLLGLGSVFSAVISQKPSRDICQRGTSLTIQCQVDSQVTMM |
| | | FWYRQQPGQSLTLIATANQGSEATYESGFVIDKFPISRPNLTFSTLTV |
| | | SNMSPEDSSIYLCSVAGTIDEQYFGPGTRLTVTE |
| 17 | Ig Cκ | RTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQ |
| | | GLSSPVTKSFNRGEC |
| 18 | IgG-CH-1a | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTK |
| | | VDKKVEPKSC |
| 19 | WinZip-B1 | SVDELQAEVDQLQDENYALKTKVAQLRKKVEKLAS |
| 20 | WinZip-A2 | TVAQLRERVKTLRAQNYELESEVQRLREQVAQLAS |
| 21 | HLA-DRA | EFDAPSPLPETTENVVCALGLTVGLVGIIIGTIFII |
| 22 | HLA-DRB1 | RARSESAQSKMLSGVGGFVLGLLFLGAGLFI |
| 23 | CD-28 | RSKRSRLLHSDYMNMTPRRPGPTRKHYQPYAPPRDFAAYRS |
| 24 | CD3ζ | RVKFSRSADAPAYQQGQNQLYNELNLGRREEYDVLDKRRGRDPE |
| | | MGGKPQRRKNPQEGLYNELQKDKMAEAYSEIGMKGERRRGKGHD |
| | | GLYQGLSTATKDTYDALHMQALPPR |
| 25 | NLV | NLVPMVATV |
| | peptide | |
| 26 | PWH308 | QKEVEQNSGPLSVPEGAIASLNCTYSDRGSQSFFWYRQYSGKSPELI |
| | | MSIYSNGDKEDGRFTAQLNKASQYVSLLIRDSQPSDSATYLCAVNV |
| | | PLSYQLTFGKGTKLSVIPNRTVAAPSVFIFPPSDEQLKSGTASVVCLL |
| | | NNFYPREAKVQWKVDNALQSGNSQESVTEQDSKDSTYSLSSTLTLS |
| | | KADYEKHKVYACEVTHQGLSSPVTKSFNRGECGSGEFDAPSPLPET |
| | | TENVVCALGLTVGLVGIIIGTIFIIRSKRSRLLHSDYMNMTPRRPGPT |
| | | RKHYQPYAPPRDFAAYRSRVKFSRSADAPAYQQGQNQLYNELNLG |
| | | RREEYDVLDKRRGRDPEMGGKPQRRKNPQEGLYNELQKDKMAEA |
| | | YSEIGMKGERRRGKGHDGLYQGLSTATKDTYDALHMQALPPR |
| 27 | PWH295 | SAVISQKPSRDICQRGTSLTIQCQVDSQVTMMFWYRQQPGQSLTLI |
| | | ATANQGSEATYESGFVIDKFPISRPNLTFSTLTVSNMSPEDSSIYLCS |
| | | VAGTIDEQYFGPGTRLTVTEGGSGGASTKGPSVFPLAPSSKSTSGGT |
| | | AALGCLVKDYFPEPVTVSWNSGALTSGVHTFPAVLQSSGLYSLSSV |
| | | VTVPSSSLGTQTYICNVNHKPSNTKVDKKVEPKSCGSGRARSESAQ |
| | | SKMLSGVGGFVLGLLFLGAGLFIYFRSKRSRLLHSDYMNMTPRRPG |
| | | PTRKHYQPYAPPRDFAAYRSRVKFSRSADAPAYQQGQNQLYNELN |
| | | LGRREEYDVLDKRRGRDPEMGGKPQRRKNPQEGLYNELQKDKMA |
| | | EAYSEIGMKGERRRGKGHDGLYQGLSTATKDTYDALHMQALPPR |
| 28 | PWH303 | SAVISQKPSRDICQRGTSLTIQCQVDSQVTMMFWYRQQPGQSLTLI |
| | | ATANQGSEATYESGFVIDKFPISRPNLTFSTLTVSNMSPEDSSIYLCS |
| | | VAGTIDEQYFGPGTRLTVTESSASTKGPSVFPLAPSSKSTSGGTAAL |
| | | GCLVKDYFPEPVTVSWNSGALTSGVHTFPAVLQSSGLYSLSSVVTV |
| | | PSSSLGTQTYICNVNHKPSNTKVDKKVEPKSCGSGRARSESAQSKM |
| | | LSGVGGFVLGLLFLGAGLFIYFRSKRSRLLHSDYMNMTPRRPGPTR |
| | | KHYQPYAPPRDFAAYRSRVKFSRSADAPAYQQGQNQLYNELNLGR |
| | | REEYDVLDKRRGRDPEMGGKPQRRKNPQEGLYNELQKDKMAEAY |
| | | SEIGMKGERRRGKGHDGLYQGLSTATKDTYDALHMQALPPR |
| 29 | PWH305 | SAVISQKPSRDICQRGTSLTIQCQVDSQVTMMFWYRQQPGQSLTLI |
| | | ATANQGSEATYESGFVIDKFPISRPNLTFSTLTVSNMSPEDSSIYLCS |
| | | VAGTIDEQYFGPGTRLTVTESSGASAPTLFPLVSCENSPSDTSSVAV |
| | | GCLAQDFLPDSITFSWKYKNNSDISSTRGFPSVLRGGKYAATSQVLL |
| | | PSKDVMQGTDEHVVCKVQHPNGNKEKNVPLPVGSGRARSESAQS |
| | | KMLSGVGGFVLGLLFLGAGLFIYFRSKRSRLLHSDYMNMTPRRPGP |
| | | TRKHYQPYAPPRDFAAYRSRVKFSRSADAPAYQQGQNQLYNELNL |
| | | GRREEYDVLDKRRGRDPEMGGKPQRRKNPQEGLYNELQKDKMAE |
| | | AYSEIGMKGERRRGKGHDGLYQGLSTATKDTYDALHMQALPPR |
| 30 | Vα-2 | QKEVEQNSGPLSVPEGAIASLNCTYSDRGSQSFFWYRQYSGKSPELI |
| | | MSIYSNGDKEDGRFTAQLNKASQYVSLLIRDSQPSDSATYLCAVNV |
| | | PLSYQLTFGKGTKLSVIPN |
| 31 | Vβ-2 | SAVISQKPSRDICQRGTSLTIQCQVDSQVTMMFWYRQQPGQSLTLI |
| | | ATANQGSEATYESGFVIDKFPISRPNLTFSTLTVSNMSPEDSSIYLCS |
| | | VAGTIDEQYFGPGTRLTVTE |
| 32 | IgG-CH-1b | SSASTKGPSVFPLAPSSKSTSGGTAALGCLVKDYFPEPVTVSWNSG |
| | | ALTSGVHTFPAVLQSSGLYSLSSVVTVPSSSLGTQTYICNVNHKPSN |
| | | TKVDKKVEPKSC |
| 33 | IgM CH-1 | SSGASAPTLFPLVSCENSPSDTSSVAVGCLAQDFLPDSITFSWKYKN |
| | | NSDISSTRGFPSVLRGGKYAATSQVLLPSKDVMQGTDEHVVCKVQ |
| | | HPNGNKEKNVPLPV |
| 34 | HLA-DRB2 | RARSESAQSKMLSGVGGFVLGLLFLGAGLFIYF |
| 35 | PWH308 | CAGAAGGAGGTGGAGCAGAATTCTGGACCCCTCAGTGTTCCAGA |
| | nucleic acid | GGGAGCCATTGCCTCTCTCAACTGCACTTACAGTGACCGAGGTT |
| | sequence | CCCAGTCCTTCTTCTGGTACAGACAATATTCTGGGAAAAGCCCT |
| | | GAGTTGATAATGTCCATATACTCCAATGGTGACAAAGAAGATGG |
| | | AAGGTTTACAGCACAGCTCAATAAAGCCAGCCAGTATGTTTCTC |
| | | TGCTCATCAGAGACTCCCAGCCCAGTGATTCAGCCACCTACCTC |
| | | TGTGCCGTGAACGTCCCCTTGAGTTACCAACTCACTTTCGGGAA |
| | | GGGGACCAAACTCTCGGTCATACCAAATAGAACTGTTGCGGCGC |
| | | CATCAGTGTTCATCTTTCCCCCTAGCGACGAGCAGCTGAAGAGT |
| | | GGCACAGCTTCCGTGGTCTGCCTGCTGAACAATTTCTACCCCCG |
| | | GGAAGCCAAGGTGCAGTGGAAAGTCGATAACGCTCTGCAGTCT |
| | | GGAAATAGTCAGGAGTCAGTGACTGAACAGGACAGCAAGGATT |
| | | CCACCTATTCTCTGAGCTCCACCCTGACACTGTCTAAAGCAGACT |
| | | ACGAGAAGCACAAAGTCTATGCCTGTGAAGTCACTCACCAGGGT |
| | | CTGTCTTCACCAGTCACCAAATCCTTCAATAGGGGGGAATGCGG |
| | | CAGTGGTGAGTTTGATGCTCCAAGCCCTCTCCCAGAGACTACAG |
| | | AGAACGTGGTGTGTGCCCTGGGCCTGACTGTGGGTCTGGTGGGC |
| | | ATCATTATTGGGACCATCTTCATCATCAGGAGTAAGAGGAGCAG |
| | | GCTCCTGCACAGTGACTACATGAACATGACTCCCCGCCGCCCCG |
| | | GGCCCACCCGCAAGCATTACCAGCCCTATGCCCCACCACGCGAC |
| | | TTCGCAGCCTATCGCTCCAGAGTGAAGTTCAGCAGGAGCGCAGA |
| | | CGCCCCCGCGTACCAGCAGGGCCAGAACCAGCTCTATAACGAGC |
| | | TCAATCTAGGACGAAGAGAGGAGTACGATGTTTTGGACAAGAG |
| | | ACGTGGCCGGGACCCTGAGATGGGGGGAAAGCCGCAGAGAAGG |
| | | AAGAACCCTCAGGAAGGCCTGTACAATGAACTGCAGAAAGATA |
| | | AGATGGCGGAGGCCTACAGTGAGATTGGGATGAAAGGCGAGCG |
| | | CCGGAGGGGCAAGGGGCACGATGGCCTTTACCAGGGTCTCAGT |
| | | ACAGCCACCAAGGACACCTACGACGCCCTTCACATGCAGGCCCT |
| | | GCCCCCTCGC |
| 36 | PWH295 | AGTGCTGTCATCTCTCAAAAGCCAAGCAGGGATATCTGTCAACG |
| | nucleic acid | TGGAACCTCCCTGACGATCCAGTGTCAAGTCGATAGCCAAGTCA |
| | sequence | CCATGATGTTCTGGTACCGTCAGCAACCTGGACAGAGCCTGACA |
| | | CTGATCGCAACTGCAAATCAGGGCTCTGAGGCCACATATGAGAG |
| | | TGGATTTGTCATTGACAAGTTTCCCATCAGCCGCCCAAACCTAA |
| | | CATTCTCAACTCTGACTGTGAGCAACATGAGCCCTGAAGACAGC |
| | | AGCATATATCTCTGCAGCGTTGCCGGGACTATCGACGAGCAGTA |
| | | CTTCGGGCCGGGCACCAGGCTCACGGTCACAGAGGGAGGATCT |
| | | GGTGGAGCTTCTACAAAAGGGCCAAGCGTGTTCCCACTGGCACC |
| | | CAGCTCCAAGTCAACCAGCGGAGGAACAGCCGCTCTGGGATGC |
| | | CTGGTGAAAGACTACTTCCCAGAGCCCGTGACCGTCTCCTGGAA |
| | | CTCTGGGGCCCTGACAAGCGGTGTGCACACTTTTCCTGCTGTCCT |
| | | GCAGTCTAGTGGGCTGTACTCCCTGTCAAGCGTGGTCACTGTGC |
| | | CATCCTCTAGTCTGGGTACTCAGACCTATATCTGCAACGTGAATC |
| | | ACAAGCCTAGCAATACCAAAGTGGACAAGAAAGTCGAACCAAA |
| | | GTCCTGTGGCAGTGGTAGAGCACGGTCTGAATCTGCACAGAGCA |
| | | AGATGCTGAGTGGAGTCGGGGGCTTTGTGCTGGGCCTGCTCTTC |
| | | CTTGGGGCCGGGCTGTTCATCTACTTCAGGAGTAAGAGGAGCAG |
| | | GCTCCTGCACAGTGACTACATGAACATGACTCCCCGCCGCCCCG |
| | | GGCCCACCCGCAAGCATTACCAGCCCTATGCCCCACCACGCGAC |
| | | TTCGCAGCCTATCGCTCCAGAGTGAAGTTCAGCAGGAGCGCAGA |
| | | CGCCCCCGCGTACCAGCAGGGCCAGAACCAGCTCTATAACGAGC |
| | | TCAATCTAGGACGAAGAGAGGAGTACGATGTTTTGGACAAGAG |
| | | ACGTGGCCGGGACCCTGAGATGGGGGGAAAGCCGCAGAGAAGG |
| | | AAGAACCCTCAGGAAGGCCTGTACAATGAACTGCAGAAAGATA |
| | | AGATGGCGGAGGCCTACAGTGAGATTGGGATGAAAGGCGAGCG |
| | | CCGGAGGGGCAAGGGGCACGATGGCCTTTACCAGGGTCTCAGT |
| | | ACAGCCACCAAGGACACCTACGACGCCCTTCACATGCAGGCCCT |
| | | GCCCCCTCGC |
| 37 | PWH303 | AGTGCTGTCATCTCTCAAAAGCCAAGCAGGGATATCTGTCAACG |
| | nucleic acid | TGGAACCTCCCTGACGATCCAGTGTCAAGTCGATAGCCAAGTCA |
| | sequence | CCATGATGTTCTGGTACCGTCAGCAACCTGGACAGAGCCTGACA |
| | | CTGATCGCAACTGCAAATCAGGGCTCTGAGGCCACATATGAGAG |
| | | TGGATTTGTCATTGACAAGTTTCCCATCAGCCGCCCAAACCTAA |
| | | CATTCTCAACTCTGACTGTGAGCAACATGAGCCCTGAAGACAGC |
| | | AGCATATATCTCTGCAGCGTTGCCGGGACTATCGACGAGCAGTA |
| | | CTTCGGGCCGGGCACCAGGCTCACGGTCACAGAGTCTAGCGCTT |
| | | CTACAAAAGGGCCAAGCGTGTTCCCACTGGCACCCAGCTCCAAG |
| | | TCAACCAGCGGAGGAACAGCCGCTCTGGGATGCCTGGTGAAAG |
| | | ACTACTTCCCAGAGCCCGTGACCGTCTCCTGGAACTCTGGGGCC |
| | | CTGACAAGCGGTGTGCACACTTTTCCTGCTGTCCTGCAGTCTAGT |
| | | GGGCTGTACTCCCTGTCAAGCGTGGTCACTGTGCCATCCTCTAGT |
| | | CTGGGTACTCAGACCTATATCTGCAACGTGAATCACAAGCCTAG |
| | | CAATACCAAAGTGGACAAGAAAGTCGAACCAAAGTCCTGTGGC |
| | | AGTGGTAGAGCACGGTCTGAATCTGCACAGAGCAAGATGCTGA |
| | | GTGGAGTCGGGGGCTTTGTGCTGGGCCTGCTCTTCCTTGGGGCC |
| | | GGGCTGTTCATCTACTTCAGGAGTAAGAGGAGCAGGCTCCTGCA |
| | | CAGTGACTACATGAACATGACTCCCCGCCGCCCCGGGCCCACCC |
| | | GCAAGCATTACCAGCCCTATGCCCCACCACGCGACTTCGCAGCC |
| | | TATCGCTCCAGAGTGAAGTTCAGCAGGAGCGCAGACGCCCCCGC |
| | | GTACCAGCAGGGCCAGAACCAGCTCTATAACGAGCTCAATCTAG |
| | | GACGAAGAGAGGAGTACGATGTTTTGGACAAGAGACGTGGCCG |
| | | GGACCCTGAGATGGGGGGAAAGCCGCAGAGAAGGAAGAACCCT |
| | | CAGGAAGGCCTGTACAATGAACTGCAGAAAGATAAGATGGCGG |
| | | AGGCCTACAGTGAGATTGGGATGAAAGGCGAGCGCCGGAGGGG |
| | | CAAGGGGCACGATGGCCTTTACCAGGGTCTCAGTACAGCCACCA |
| | | AGGACACCTACGACGCCCTTCACATGCAGGCCCTGCCCCCTCGC |
| 38 | PWH305 | AGTGCTGTCATCTCTCAAAAGCCAAGCAGGGATATCTGTCAACG |
| | nucleic acid | TGGAACCTCCCTGACGATCCAGTGTCAAGTCGATAGCCAAGTCA |
| | sequence | CCATGATGTTCTGGTACCGTCAGCAACCTGGACAGAGCCTGACA |
| | | CTGATCGCAACTGCAAATCAGGGCTCTGAGGCCACATATGAGAG |
| | | TGGATTTGTCATTGACAAGTTTCCCATCAGCCGCCCAAACCTAA |
| | | CATTCTCAACTCTGACTGTGAGCAACATGAGCCCTGAAGACAGC |
| | | AGCATATATCTCTGCAGCGTTGCCGGGACTATCGACGAGCAGTA |
| | | CTTCGGGCCGGGCACCAGGCTCACGGTCACAGAGtctagcggaAGTG |
| | | CTAGCGCCCCAACCCTTTTCCCCCTCGTCTCCTGTGAGAATTCCC |
| | | CGTCGGATACGAGCAGCGTGGCCGTTGGCTGCCTCGCACAGGAC |
| | | TTCCTTCCCGACTCCATCACTTTCTCCTGGAAATACAAGAACAAC |
| | | TCTGACATCAGCAGCACCCGGGGCTTCCCATCAGTCCTGAGAGG |
| | | GGGCAAGTACGCAGCCACCTCACAGGTGCTGCTGCCTTCCAAGG |
| | | ACGTCATGCAGGGCACAGACGAACACGTGGTGTGCAAAGTCCA |
| | | GCACCCCAACGGCAACAAAGAAAAGAACGTGCCTCTTCCAGGC |
| | | AGTGGTAGAGCACGGTCTGAATCTGCACAGAGCAAGATGCTGA |
| | | GTGGAGTCGGGGGCTTTGTGCTGGGCCTGCTCTTCCTTGGGGCC |
| | | GGGCTGTTCATCTACTTCAGGAGTAAGAGGAGCAGGCTCCTGCA |
| | | CAGTGACTACATGAACATGACTCCCCGCCGCCCCGGGCCCACCC |
| | | GCAAGCATTACCAGCCCTATGCCCCACCACGCGACTTCGCAGCC |
| | | TATCGCTCCAGAGTGAAGTTCAGCAGGAGCGCAGACGCCCCCGC |
| | | GTACCAGCAGGGCCAGAACCAGCTCTATAACGAGCTCAATCTAG |
| | | GACGAAGAGAGGAGTACGATGTTTTGGACAAGAGACGTGGCCG |
| | | GGACCCTGAGATGGGGGGAAAGCCGCAGAGAAGGAAGAACCCT |
| | | CAGGAAGGCCTGTACAATGAACTGCAGAAAGATAAGATGGCGG |
| | | AGGCCTACAGTGAGATTGGGATGAAAGGCGAGCGCCGGAGGGG |
| | | CAAGGGGCACGATGGCCTTTACCAGGGTCTCAGTACAGCCACCA |
| | | AGGACACCTACGACGCCCTTCACATGCAGGCCCTGCCCCCTCGC |
| 39 | P-NR-025 | RTVAAPSVFIFPPSDEQLKSGTASVVCLLNNFYPREAKVQWKVDNA |
| | constant- | LQSGNSQESVTEQDSKDSTYSLSSTLTLSKADYEKHKVYACEVTHQ |
| | connecting | GLSSPVTKSFNRGECGSGEFDAPSPLPETTENVVCALGLTVGLVGIII |
| | peptide- | GTIFII |
| | trans- | |
| | membrane | |
| | domains | |
| 40 | P-NR-026 | ASTKGPSVFPLAPSSKSTSGGTAALGCLVKDYFPEPVTVSWNSGAL |
| | constant- | TSGVHTFPAVLQSSGLYSLSSVVTVPSSSLGTQTYICNVNHKPSNTK |
| | connecting | VDKKVEPKSCGSGRARSESAQSKMLSGVGGFVLGLLFLGAGLFI |
| | peptide- | |
| | trans- | |
| | membrane | |
| | domains | |
| 41 | P-NR-027 | SVDELQAEVDQLQDENYALKTKVAQLRKKVEKLASGGCGGEFDA |
| | constant- | PSPLPETTENVVCALGLTVGLVGIIIGTIFII |
| | connecting | |
| | peptide- | |
| | trans- | |
| | membrane | |
| | domains | |
| 42 | P-NR-028 | TVAQLRERVKTLRAQNYELESEVQRLREQVAQLASGGCGGRARSE |
| | constant- | SAQSKMLSGVGGFVLGLLFLGAGLFI |
| | connecting | |
| | peptide- | |
| | trans- | |
| | membrane | |
| | domains | |
| 43 | PWH308 | RTVAAPSVFIFPPSDEQLKSGTASVVCLLNNFYPREAKVQWKVDNA |
| | constant- | LQSGNSQESVTEQDSKDSTYSLSSTLTLSKADYEKHKVYACEVTHQ |
| | connecting | GLSSPVTKSFNRGECGSGEFDAPSPLPETTENVVCALGLTVGLVGIII |
| | peptide- | GTIFII |
| | trans- | |
| | membrane | |
| | domains | |
| 44 | PWH295 | ASTKGPSVFPLAPSSKSTSGGTAALGCLVKDYFPEPVTVSWNSGAL |
| | constant- | TSGVHTFPAVLQSSGLYSLSSVVTVPSSSLGTQTYICNVNHKPSNTK |
| | connecting | VDKKVEPKSCGSGRARSESAQSKMLSGVGGFVLGLLFLGAGLFIYF |
| | peptide- | |
| | trans- | |
| | membrane | |
| | domains | |
| 45 | PWH303 | SSASTKGPSVFPLAPSSKSTSGGTAALGCLVKDYFPEPVTVSWNSG |
| | constant- | ALTSGVHTFPAVLQSSGLYSLSSVVTVPSSSLGTQTYICNVNHKPSN |
| | connecting | TKVDKKVEPKSCGSGRARSESAQSKMLSGVGGFVLGLLFLGAGLFI |
| | peptide- | YF |
| | trans- | |
| | membrane | |
| | domains | |
| 46 | PWH305 | SSGASAPTLFPLVSCENSPSDTSSVAVGCLAQDFLPDSITFSWKYKN |
| | constant- | NSDISSTRGFPSVLRGGKYAATSQVLLPSKDVMQGTDEHVVCKVQ |
| | connecting | HPNGNKEKNVPLPVGSGRARSESAQSKMLSGVGGFVLGLLFLGAG |
| | peptide- | LFIYF |
| | trans- | |
| | membrane | |
| | domains | |

1. A modified T cell receptor (TCR) comprising a first peptide chain and a second peptide chain, wherein each peptide chain comprises:
an extracellular domain;
a transmembrane domain; and
an intracellular domain,
wherein the extracellular domain comprises a variable region, a constant region, and a connecting peptide,
wherein the variable region and the constant region are attached via a linker, wherein the constant region of the first peptide chain comprises an Ig-Cκ domain and the constant region of the second peptide chain comprises an Ig-CH-1 domain, and
wherein either 1) the transmembrane domain of the first peptide chain comprises an HLA-DRA domain and the transmembrane domain of the second peptide chain comprises an HLA-DRB domain, or 2) the transmembrane domain of the first peptide chain comprises an HLA-DRB domain and the transmembrane domain of the second peptide chain comprises an HLA-DRA domain.

2. The modified TCR of claim 1, wherein the linker is a flexible linker.

3. The modified TCR of claim 1, wherein the Ig-CH-1 domain is IgG-CH-1a, IgG-CH-1b, or IgM CH-1.

4. The modified TCR of claim 1, wherein the HLA-DRB domain is HLA-DRB1 or HLA-DRB2.

5. The modified TCR of claim 1, wherein the variable regions on each of the peptide chains are the same variable region.

6. The modified TCR of claim 1, wherein the variable regions on each peptide chain are different from each other.

7. The modified TCR of claim 1, wherein the intracellular domain comprises a CD28 region and a CD3ξ immunoreceptor tyrosine-based activation motifs (ITAM) region.

8. The modified TCR of claim 1, wherein the first peptide chain comprises:
Ig-Cκ as the constant region;
HLA-DRA as the transmembrane domain; and
CD28 and CD3ξ as the intracellular domain.

9. The modified TCR of claim 8, wherein the first peptide chain comprises SEQ ID NO: 39 or SEQ ID NO: 43.

10. (canceled)

11. The modified TCR of claim 1, wherein the second peptide chain comprises:
Ig-CH-1 as the constant region;
HLA-DRB as the transmembrane domain; and
CD28 and CD3ξ as the intracellular domain.

12. The modified TCR of claim 11, wherein the second peptide chain comprises SEQ ID NO: 40, SEQ ID NO: 44. SEQ ID NO: 45, or SEQ ID NO: 46.

13.-15. (canceled)

16. The modified TCR of any-m 1, wherein
the first peptide chain comprises Ig-Cκ as the constant region, HLA-DRA as the transmembrane domain; and CD28 and CD35 as the intracellular domain; and
the second peptide chain comprises Ig-CH-1 as the constant region, HLA-DRB as the transmembrane domain, and CD28 and CD35 as the intracellular domain.

17. The modified TCR of claim 16, wherein the first peptide chain comprises SEQ ID NO: 39 and the second peptide chain comprises SEQ ID NO: 40.

18. The modified TCR of claim 17, wherein the first peptide chain further comprises SEQ ID NO: 15 and the second peptide chain further comprises SEQ ID NO: 16.

19. The modified TCR of claim 16, wherein the first peptide chain comprises SEQ ID NO: 43 and the second peptide chain comprises SEQ ID NO: 44.

20. The modified TCR of claim 19, wherein the first peptide chain further comprises SEQ ID NO: 30 and the second peptide chain further comprises SEQ ID NO: 31.

21.-24. (canceled)

25. A cell comprising the modified TCR of a claim 1.

26. A nucleic acid encoding the modified TCR of claim 1.

27. A vector comprising the nucleic acid of claim 26.

28. A method for reducing the occurrence of or treating cancer or a viral infection in a patient in need thereof, the method comprising administering a pharmaceutical composition to the patient, wherein the pharmaceutical composition comprises a therapeutically effective amount of the modified TCR of claim 1 or a nucleic acid encoding the modified TCR.

29. The method of claim 28, wherein the pharmaceutical comprises a vector that comprises the nucleic acid.

30. The method of claim 28, wherein the pharmaceutical composition comprises a cell comprising the modified TCR or the nucleic acid encoding the modified TCR.

31. The method of claim 28, wherein the cancer is selected from the group consisting of bladder cancer, bone cancer, brain cancer, breast cancer, cancer of the central nervous system, cervical cancer, colon cancer, colorectal cancer, esophageal cancer, eye cancer, gall bladder cancer, head and neck cancer, gastric cancer, HIV/AIDS related cancer, kidney cancer, leukemia, liver cancer, lung cancer, lymphoma, melanoma, multiple myeloma, nasopharyngeal cancer, oral cancer, neuroendocrine cancer, ovarian cancer, pancreatic cancer, prostate cancer, retinoblastoma, sarcoma, skin cancer, stomach cancer, testicular cancer, thyroid cancer, uterine cancer, and vaginal cancer.

32. The method of claim 28, wherein the viral infection is caused by a virus from a viral family selected from the group consisting of herpesviridae, adenoviridae, polyomavididac, poxviridae, reoviridae, coronaviridae, picomaviridac, flaviviridac, hepeviridac, togaviridac, Miloviridac, paramyxoviridae, pneumoviridac, rhabdoviridae, hantaviridac, and orthomyxoviride.

33. (canceled)


SEQUENCE LISTING (46 sequences)

SEQ ID NO: 1 - protein, 5 aa, synthetic construct
GGCGG

SEQ ID NO: 2 - protein, 5 aa, synthetic construct
GGSGG

SEQ ID NO: 3 - protein, 377 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNVPLSYQLTFGKGTKLSVIPNAGSSTGSSTGPGSTSVDELQAEVDQLQDENYALKTKVAQLRKKVEKLASGGCGGEFDAPSPLPETTENVVCALGLTVGLVGIIIGTIFIIRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 4 - protein, 368 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVAGTIDEQYFGPGTRLTVTESGGTSGSTSGTGSTTVAQLRERVKTLRAQNYELESEVQRLREQVAQLASGGCGGRARSESAQSKMLSGVGGFVLGLLFLGAGLFIRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 438 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNVPLSYQLTFGKGTKLSVIPNGGSGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGEFDAPSPLPETTENVVCALGLTVGLVGIIIGTIFIIRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 425 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVAGTIDEQYFGPGTRLTVTEGGSGGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSGRARSESAQSKMLSGVGGFVLGLLFLGAGLFIRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - DNA, 7237 bp, synthetic construct
  source 1..7237: mol_type = other DNA
agctagctttaatacgactcactataaggaaataagagagaaaagaagagtaagaagaaatataagagccaccatgggctagtacagatatctggtggaagaactgttgcggcgccatcagtgttcatctttccccctagcgacgagcagctgaagagtggcacagcttccgtggtctgcctgctgaacaatttctacccccgggaagccaaggtgcagtggaaagtcgataacgctctgcagtctggaaatagtcaggagtcagtgactgaacaggacagcaaggattccacctattctctgagctccaccctgacactgtctaaagcagactacgagaagcacaaagtctatgcctgtgaagtcactcaccagggtctgtcttcaccagtcaccaaatccttcaataggggggaatgcggcagtggtgagtttgatgctccaagccctctcccagagactacagagaacgtggtgtgtgccctgggcctgactgtgggtctggtgggcatcattattgggaccatcttcatcatcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagaaaaaaaaaaaaaaaaaaaaaaagcaggtggcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaa

SEQ ID NO: 8 - DNA, 7625 bp, synthetic construct
  source 1..7625: mol_type = other DNA
agctagctttaatacgactcactataaggaaataagagagaaaagaagagtaagaagaaatataagagccaccatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaacgtccccttgagttaccaactcactttcgggaaggggaccaaactctcggtcataccaaatggaggatctggtggaagaactgttgcggcgccatcagtgttcatctttccccctagcgacgagcagctgaagagtggcacagcttccgtggtctgcctgctgaacaatttctacccccgggaagccaaggtgcagtggaaagtcgataacgctctgcagtctggaaatagtcaggagtcagtgactgaacaggacagcaaggattccacctattctctgagctccaccctgacactgtctaaagcagactacgagaagcacaaagtctatgcctgtgaagtcactcaccagggtctgtcttcaccagtcaccaaatccttcaataggggggaatgcggcagtggtgagtttgatgctccaagccctctcccagagactacagagaacgtggtgtgtgccctgggcctgactgtgggtctggtgggcatcattattgggaccatcttcatcatcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagaaaaaaaaaaaaaaaaaaaaaaagcaggtggcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaa

SEQ ID NO: 9 - DNA, 7210 bp, synthetic construct
  source 1..7210: mol_type = other DNA
agctagctttaatacgactcactataaggaaataagagagaaaagaagagtaagaagaaatataagagccaccatgggctagtacagatatctggtggagcttctacaaaagggccaagcgtgttcccactggcacccagctccaagtcaaccagcggaggaacagccgctctgggatgcctggtgaaagactacttcccagagcccgtgaccgtctcctggaactctggggccctgacaagcggtgtgcacacttttcctgctgtcctgcagtctagtgggctgtactccctgtcaagcgtggtcactgtgccatcctctagtctgggtactcagacctatatctgcaacgtgaatcacaagcctagcaataccaaagtggacaagaaagtcgaaccaaagtcctgtggcagtggtagagcacggtctgaatctgcacagagcaagatgctgagtggagtcgggggctttgtgctgggcctgctcttccttggggccgggctgttcatcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagaaaaaaaaaaaaaaaaaaaaaaagcaggtggcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaa

SEQ ID NO: 10 - DNA, 7586 bp, synthetic construct
  source 1..7586: mol_type = other DNA
agctagctttaatacgactcactataaggaaataagagagaaaagaagagtaagaagaaatataagagccaccatgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgccgggactatcgacgagcagtacttcgggccgggcaccaggctcacggtcacagagggaggatctggtggagcttctacaaaagggccaagcgtgttcccactggcacccagctccaagtcaaccagcggaggaacagccgctctgggatgcctggtgaaagactacttcccagagcccgtgaccgtctcctggaactctggggccctgacaagcggtgtgcacacttttcctgctgtcctgcagtctagtgggctgtactccctgtcaagcgtggtcactgtgccatcctctagtctgggtactcagacctatatctgcaacgtgaatcacaagcctagcaataccaaagtggacaagaaagtcgaaccaaagtcctgtggcagtggtagagcacggtctgaatctgcacagagcaagatgctgagtggagtcgggggctttgtgctgggcctgctcttccttggggccgggctgttcatcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagaaaaaaaaaaaaaaaaaaaaaaagcaggtggcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaa

SEQ ID NO: 11 - DNA, 7054 bp, synthetic construct
  source 1..7054: mol_type = other DNA
agctagctttaatacgactcactataaggaaataagagagaaaagaagagtaagaagaaatataagagccaccatgggctagtacagatatctagtacaggtagctccactgggccgggtagcacatcagttgatgagttgcaggccgaggtggaccagcttcaagatgagaactacgctctgaaaacgaaagtagcacagttgcgaaaaaaggtagagaagctcgcgtctggtggatgtggtggagagtttgatgctccaagccctctcccagagactacagagaacgtggtgtgtgccctgggcctgactgtgggtctggtgggcatcattattgggaccatcttcatcatcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagaaaaaaaaaaaaaaaaaaaaaaagcaggtggcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaa

SEQ ID NO: 12 - DNA, 7442 bp, synthetic construct
  source 1..7442: mol_type = other DNA
agctagctttaatacgactcactataaggaaataagagagaaaagaagagtaagaagaaatataagagccaccatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaacgtccccttgagttaccaactcactttcgggaaggggaccaaactctcggtcataccaaatgccggatctagtacaggtagctccactgggccgggtagcacatcagttgatgagttgcaggccgaggtggaccagcttcaagatgagaactacgctctgaaaacgaaagtagcacagttgcgaaaaaaggtagagaagctcgcgtctggtggatgtggtggagagtttgatgctccaagccctctcccagagactacagagaacgtggtgtgtgccctgggcctgactgtgggtctggtgggcatcattattgggaccatcttcatcatcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagaaaaaaaaaaaaaaaaaaaaaaagcaggtggcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaa

SEQ ID NO: 13 - DNA, 7033 bp, synthetic construct
  source 1..7033: mol_type = other DNA
agctagctttaatacgactcactataaggaaataagagagaaaagaagagtaagaagaaatataagagccaccatgggctagtacagatatcaggatctaccagcggcacgggctctactacggtggcccaactcagggaacgggtcaagacactgagagcccaaaattacgaattggagtctgaggttcaacgccttcgagagcaagttgcgcagcttgcaagtggtggatgtggtggaagagcacggtctgaatctgcacagagcaagatgctgagtggagtcgggggctttgtgctgggcctgctcttccttggggccgggctgttcatcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagaaaaaaaaaaaaaaaaaaaaaaagcaggtggcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaa

SEQ ID NO: 14 - DNA, 7415 bp, synthetic construct
  source 1..7415: mol_type = other DNA
agctagctttaatacgactcactataaggaaataagagagaaaagaagagtaagaagaaatataagagccaccatgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgccgggactatcgacgagcagtacttcgggccgggcaccaggctcacggtcacagagagtgggggcacatcaggatctaccagcggcacgggctctactacggtggcccaactcagggaacgggtcaagacactgagagcccaaaattacgaattggagtctgaggttcaacgccttcgagagcaagttgcgcagcttgcaagtggtggatgtggtggaagagcacggtctgaatctgcacagagcaagatgctgagtggagtcgggggctttgtgctgggcctgctcttccttggggccgggctgttcatcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagaaaaaaaaaaaaaaaaaaaaaaagcaggtggcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaa

SEQ ID NO: 15 - protein, 132 aa, synthetic construct
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVNVPLSYQLTFGKGTKLSVIP

SEQ ID NO: 16 - protein, 129 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVAGTIDEQYFGPGTRLTVTE

SEQ ID NO: 17 - protein, 107 aa, synthetic construct [sequence omitted]

SEQ ID NO: 18 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 35 aa, synthetic construct
SVDELQAEVDQLQDENYALKTKVAQLRKKVEKLAS

SEQ ID NO: 20 - protein, 35 aa, synthetic construct
TVAQLRERVKTLRAQNYELESEVQRLREQVAQLAS

SEQ ID NO: 21 - protein, 36 aa, synthetic construct
EFDAPSPLPETTENVVCALGLTVGLVGIIIGTIFII

SEQ ID NO: 22 - protein, 31 aa, synthetic construct
RARSESAQSKMLSGVGGFVLGLLFLGAGLFI

SEQ ID NO: 23 - protein, 41 aa, synthetic construct
RSKRSRLLHSDYMNMTPRRPGPTRKHYQPYAPPRDFAAYRS

SEQ ID NO: 24 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 9 aa, synthetic construct
NLVPMVATV

SEQ ID NO: 26 - protein, 412 aa, synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAVNVPLSYQLTFGKGTKLSVIPNRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGEFDAPSPLPETTENVVCALGLTVGLVGIIIGTIFIIRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 27 - protein, 412 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVAGTIDEQYFGPGTRLTVTEGGSGGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSGRARSESAQSKMLSGVGGFVLGLLFLGAGLFIYFRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 409 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVAGTIDEQYFGPGTRLTVTESSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSGRARSESAQSKMLSGVGGFVLGLLFLGAGLFIYFRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 29 - protein, 410 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVAGTIDEQYFGPGTRLTVTESSGASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVGSGRARSESAQSKMLSGVGGFVLGLLFLGAGLFIYFRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - protein, 112 aa, synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAVNVPLSYQLTFGKGTKLSVIPN

SEQ ID NO: 31 - protein, 114 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSVAGTIDEQYFGPGTRLTVTE

SEQ ID NO: 32 - protein, 105 aa, synthetic construct
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 106 aa, synthetic construct
SSGASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPV

SEQ ID NO: 34 - protein, 33 aa, synthetic construct
RARSESAQSKMLSGVGGFVLGLLFLGAGLFIYF

SEQ ID NO: 35 - DNA, 1236 bp, synthetic construct
  source 1..1236: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaacgtccccttgagttaccaactcactttcgggaaggggaccaaactctcggtcataccaaatagaactgttgcggcgccatcagtgttcatctttccccctagcgacgagcagctgaagagtggcacagcttccgtggtctgcctgctgaacaatttctacccccgggaagccaaggtgcagtggaaagtcgataacgctctgcagtctggaaatagtcaggagtcagtgactgaacaggacagcaaggattccacctattctctgagctccaccctgacactgtctaaagcagactacgagaagcacaaagtctatgcctgtgaagtcactcaccagggtctgtcttcaccagtcaccaaatccttcaataggggggaatgcggcagtggtgagtttgatgctccaagccctctcccagagactacagagaacgtggtgtgtgccctgggcctgactgtgggtctggtgggcatcattattgggaccatcttcatcatcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - DNA, 1236 bp, synthetic construct
  source 1..1236: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgccgggactatcgacgagcagtacttcgggccgggcaccaggctcacggtcacagagggaggatctggtggagcttctacaaaagggccaagcgtgttcccactggcacccagctccaagtcaaccagcggaggaacagccgctctgggatgcctggtgaaagactacttcccagagcccgtgaccgtctcctggaactctggggccctgacaagcggtgtgcacacttttcctgctgtcctgcagtctagtgggctgtactccctgtcaagcgtggtcactgtgccatcctctagtctgggtactcagacctatatctgcaacgtgaatcacaagcctagcaataccaaagtggacaagaaagtcgaaccaaagtcctgtggcagtggtagagcacggtctgaatctgcacagagcaagatgctgagtggagtcgggggctttgtgctgggcctgctcttccttggggccgggctgttcatctacttcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 37 - DNA, 1227 bp, synthetic construct
  source 1..1227: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgccgggactatcgacgagcagtacttcgggccgggcaccaggctcacggtcacagagtctagcgcttctacaaaagggccaagcgtgttcccactggcacccagctccaagtcaaccagcggaggaacagccgctctgggatgcctggtgaaagactacttcccagagcccgtgaccgtctcctggaactctggggccctgacaagcggtgtgcacacttttcctgctgtcctgcagtctagtgggctgtactccctgtcaagcgtggtcactgtgccatcctctagtctgggtactcagacctatatctgcaacgtgaatcacaagcctagcaataccaaagtggacaagaaagtcgaaccaaagtcctgtggcagtggtagagcacggtctgaatctgcacagagcaagatgctgagtggagtcgggggctttgtgctgggcctgctcttccttggggccgggctgttcatctacttcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 38 - DNA, 1230 bp, synthetic construct
  source 1..1230: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgccgggactatcgacgagcagtacttcgggccgggcaccaggctcacggtcacagagtctagcggaagtgctagcgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccaggcagtggtagagcacggtctgaatctgcacagagcaagatgctgagtggagtcgggggctttgtgctgggcctgctcttccttggggccgggctgttcatctacttcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 39 - protein, 146 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGEFDAPSPLPETTENVVCALGLTVGLVGIIIGTIFII

SEQ ID NO: 40 - protein, 137 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSGRARSESAQSKMLSGVGGFVLGLLFLGAGLFI

SEQ ID NO: 41 - protein, 76 aa, synthetic construct
SVDELQAEVDQLQDENYALKTKVAQLRKKVEKLASGGCGGEFDAPSPLPETTENVVCALGLTVGLVGIIIGTIFII

SEQ ID NO: 42 - protein, 71 aa, synthetic construct
TVAQLRERVKTLRAQNYELESEVQRLREQVAQLASGGCGGRARSESAQSKMLSGVGGFVLGLLFLGAGLFI

SEQ ID NO: 43 - protein, 146 aa, synthetic construct; identical to SEQ ID NO: 39

SEQ ID NO: 44 - protein, 139 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSGRARSESAQSKMLSGVGGFVLGLLFLGAGLFIYF

SEQ ID NO: 45 - protein, 141 aa, synthetic construct
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSGRARSESAQSKMLSGVGGFVLGLLFLGAGLFIYF

SEQ ID NO: 46 - protein, 142 aa, synthetic construct
SSGASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVGSGRARSESAQSKMLSGVGGFVLGLLFLGAGLFIYF